SEQUENCE LISTING (67 sequences)

SEQ ID NO: 1 - protein, 33 aa, Artificial Sequence
  MMRN2 conserved region consensus sequence
  VARIANT 1: Any amino acid
  VARIANT 2: V or L
  VARIANT 3..4: Any amino acids
  VARIANT 6..8: Any amino acids
  VARIANT 10..11: Any amino acids
  VARIANT 14: Any amino acid
  VARIANT 17: Any amino acid
  VARIANT 20..22: Any amino acids
  VARIANT 24..25: Any amino acids
  VARIANT 27: Any amino acid
  VARIANT 31..33: Any amino acids
XXXXLXXXFXXLLXDAXRHXXXLXXLXGEEXXX

SEQ ID NO: 2 - protein, 33 aa, Artificial Sequence; identical to SEQ ID NO: 1
  Preferred embodiment of MMRN2 conserved region consensus sequence
  VARIANT 1: any basic amino acid, such as E, D, K or H
  VARIANT 2: V or L
  VARIANT 3: R or K or Q
  VARIANT 4: Any amino acid
  VARIANT 6: H or E or S or N
  VARIANT 7: S or G or A
  VARIANT 8: A or S or T
  VARIANT 10: Any amino acid
  VARIANT 11: A or T or S
  VARIANT 14: E or Q or N
  VARIANT 17: L or T or V or M
  VARIANT 20: E or Q or S
  VARIANT 21: A or D or E
  VARIANT 22: V or A
  VARIANT 24: A or E
  VARIANT 25: A or I or V
  VARIANT 27: F or L
  VARIANT 31: V or M or F
  VARIANT 32: L or M or V or I
  VARIANT 33: E or D

SEQ ID NO: 3 - protein, 33 aa, Artificial Sequence
  Particularly preferred embodiment of MMRN2 conserved region consensus sequence
  VARIANT 2: V or L
  VARIANT 4: Any amino acid (preferably Q)
  VARIANT 10: Any amino acid (preferably A)
EXRXLHSAFXALLEDALRHEAVLAALFGEEVLE

SEQ ID NO: 4 - protein, 24 aa, Homo sapiens
  Oncostatin M leader sequence
MGVLLTQRTLLSLVLALLFPSMAS

SEQ ID NO: 5 - protein, 12 aa, Homo sapiens
  CLEC14A residues 97-108
ERRRSHCTLENE

SEQ ID NO: 6 - DNA, 38 bp, Artificial Sequence
  CLEC14A wt forward primer
gatctcgagctcaagcttcgatgaggccggcgttcgcc

SEQ ID NO: 7 - DNA, 33 bp, Artificial Sequence
  CLEC14A wt reverse primer
taccgtcgactgcagtgcatcactagagccaag

SEQ ID NO: 8 - DNA, 33 bp, Artificial Sequence
  CD93 wt forward primer
cgagctcaagcttcgatggccacctccatgggc

SEQ ID NO: 9 - DNA, 35 bp, Artificial Sequence
  CD93 wt reverse primer
taccgtcgactgcaggcagtctgtcccaggtgtcg

SEQ ID NO: 10 - DNA, 33 bp, Artificial Sequence
  THBD wt forward primer
cgagctcaagcttcgatgcttggggtcctggtc

SEQ ID NO: 11 - DNA, 33 bp, Artificial Sequence
  THBD wt reverse primer
taccgtcgactgcaggagtctctgcggcgtccg

SEQ ID NO: 12 - DNA, 27 bp, Artificial Sequence
  THBD-CTLD fused to CLEC14A-sushi reverse primer
ctcaaactggaactcgcagaggaagcc

SEQ ID NO: 13 - DNA, 27 bp, Artificial Sequence
  THBD-CTLD fused to CLEC14A-sushi forward primer
gcgagttccagtttgaggtcttgtgtc

SEQ ID NO: 14 - DNA, 33 bp, Artificial Sequence; identical to SEQ ID NO: 7
  CLEC14A-CTLD fused to THBD-sushi reverse primer

SEQ ID NO: 15 - DNA, 32 bp, Artificial Sequence
  CLEC14A-CTLD fused to THBD-sushi forward primer
gtgcaagtaccacttcccagccacctgcaggc

SEQ ID NO: 16 - DNA, 28 bp, Artificial Sequence
  THBD-sushi fused to CLEC14A-E.G.F reverse primer
tcccggggcaagcgcccggcgcctccct

SEQ ID NO: 17 - DNA, 28 bp, Artificial Sequence
  THBD-sushi fused to CLEC14A-E.G.F forward primer
gccgggcgcttgccccgggaggtacctc

SEQ ID NO: 18 - DNA, 36 bp, Artificial Sequence
  CLEC14A C103S reverse primer
ctcgttctccagggttgagtgggaacgcctgcgctc

SEQ ID NO: 19 - DNA, 36 bp, Artificial Sequence
  CLEC14A C103S forward primer
gagcgcaggcgttcccactcaaccctggagaacgag

SEQ ID NO: 20 - DNA, 39 bp, Artificial Sequence
  CLEC14A C138S reverse primer
cgcgcatctccgcgcggtggaggagcgttggggctcctc

SEQ ID NO: 21 - DNA, 39 bp, Artificial Sequence
  CLEC14A C138S forward primer
gaggagccccaacgctcctccaccgcgcggagatgcgcg

SEQ ID NO: 22 - DNA, 29 bp, Artificial Sequence
  MMRN2 FL Forward primer
ccggaccggtcaggcttccagtactagcc

SEQ ID NO: 23 - DNA, 28 bp, Artificial Sequence
  MMRN2 820 Reverse primer
ctactaggtaccccagagcgccgcgccc

SEQ ID NO: 24 - DNA, 30 bp, Artificial Sequence
  MMRN2 133 Forward primer
ccggaccggtgattccatggcaatccctga

SEQ ID NO: 25 - DNA, 29 bp, Artificial Sequence
  MMRN2 FL Reverse primer
cggggtaccggtcttaaacatcaggaagc

SEQ ID NO: 26 - DNA, 30 bp, Artificial Sequence
  MMRN2 487 Forward primer
ccggaccggttacgtgaaggactgcaattg

SEQ ID NO: 27 - DNA, 29 bp, Artificial Sequence
  MMRN2 674 Reverse primer
ctactaggtacccggccgcgggggctccg

SEQ ID NO: 28 - DNA, 27 bp, Artificial Sequence
  MMRN2 675 Forward primer
ccggaccggtgcagagcacctggagcc

SEQ ID NO: 29 - DNA, 29 bp, Artificial Sequence
  MMRN2 603 Reverse primer
ctactaggtacccgcgtcctccagcaggg

SEQ ID NO: 30 - DNA, 28 bp, Artificial Sequence
  MMRN2 604 Forward primer
ccggaccggtctgcggcacgaggcggtg

SEQ ID NO: 31 - DNA, 28 bp, Artificial Sequence
  MMRN2 530 Forward primer
ccggaccggtggctcctccctgcaggcc

SEQ ID NO: 32 - DNA, 31 bp, Artificial Sequence
  MMRN2 624 Reverse primer
ctactaggtaccctcagacatctcctccagc

SEQ ID NO: 33 - DNA, 33 bp, Artificial Sequence
  MMRN2 495 Forward primer
tagtagaccggtcagaagctctatttagacctg

SEQ ID NO: 34 - DNA, 31 bp, Artificial Sequence
  mouse MMRN2 495 Forward primer
ccggaccggtcaaagggtcaactctgacgtg

SEQ ID NO: 35 - DNA, 31 bp, Artificial Sequence
  mouse MMRN2 678 Reverse primer
ctactaggtacccaactgtgggtgctgctcc

SEQ ID NO: 36 - DNA, 45 bp, Artificial Sequence
  BirA
ggtggtggtctgaacgatatttttgaagctcagaaaatcgaatgg

SEQ ID NO: 37 - DNA, 38 bp, Artificial Sequence
  Mouse CLEC14A-ECD forward primer
actagcctcgaggtttaaacatgaggccagcgcttgcc

SEQ ID NO: 38 - DNA, 43 bp, Artificial Sequence
  Mouse CLEC14A-ECD reverse primer
cactcgatgaggatccggaagaggtgtcgaaagtcagagaaac

SEQ ID NO: 39 - DNA, 41 bp, Artificial Sequence
  mouse Fc for fusion to mCLEC14A forward primer
cctcttccggatcctcatcgagtgtgcccagggattgtggt

SEQ ID NO: 40 - DNA, 36 bp, Artificial Sequence
  mouse Fc for fusion to mCLEC14A reverse primer
ctgcagcccgtagttttcatttaccaggagagtggg

SEQ ID NO: 41 - DNA, 39 bp, Artificial Sequence
  mouse CLEC14A signal peptide forward primer
agactagcctcgaggtttaaacatgaggccagcgcttgc

SEQ ID NO: 42 - DNA, 27 bp, Artificial Sequence
  mouse CLEC14A signal peptide reverse primer
tgaggatccctccccattccctggccg

SEQ ID NO: 43 - DNA, 27 bp, Artificial Sequence
  mouse Fc fused to signal peptide forward primer
aatggggagggatcctcatcgagtgtg

SEQ ID NO: 44 - DNA, 37 bp, Artificial Sequence
  mouse Fc fused to signal peptide reverse primer
tcctgcagcccgtagttttcatttaccaggagagtgg

SEQ ID NO: 45 - DNA, 39 bp, Artificial Sequence
  Mouse MMRN2 495-678 forward primer
cagggaatggggagggatcccaaagggtcaactctgacg

SEQ ID NO: 46 - DNA, 38 bp, Artificial Sequence
  Mouse MMRN2 495-678 reverse primer
ggcacactcgatgaggatcccaactgtgggtgctgctc

SEQ ID NO: 47 - protein, 180 aa, Homo sapiens
  MMRN2 495-674
QKLYLDLDVIREGQRDATRALEETQVSLDERRQLDGSSLQALQNAVDAVSLAVDAHKAEGERARAATSRLRSQVQALDDEVGALKAAAAEARHEVRQLHSAFAALLEDALRHEAVLAALFGEEVLEEMSEQTPGPLPLSYEQIRVALQDAASGLQEQALGWDELAARVTALEQASEPPRP

SEQ ID NO: 48 - protein, 181 aa, Bos taurus
  MMRN2 492-672
QKLYFDLDVIREDQRDTTRALEETQVTLDERRQQDGSSLQALRDTVASLSLAVDTQQAEGERARAEAARLRSQLRALGGEVSALQAAETEIRRDIRQLHSSFSALLEDALRHEAVLAALFGEEVMEEMSEEGPGTLPLRYEQIRVALMDAASGLQEQALGWDALAARVTALEQASGARRQT

SEQ ID NO: 49 - protein, 180 aa, Pan troglodytes; identical to SEQ ID NO: 47
  MMRN2 495-674

SEQ ID NO: 50 - protein, 183 aa, Rattus norvegicus
  Mmrn2 490-672
RRVYSDMDVIPEGRRDAMHTLEETQVSPDEQHQLDGSLQALQSTVDAMSSALDAYRGEGERARAERARMRSQLRALDHAVEALKTAANGTRKEIRLLHGSFAALLEDALRHQAVLAALFGEEMVDEMSEEPPRPLPLNYEQIRLALQDAASGLQEQAVGWEDLATRVEALEKAAGGFVEQHPR

SEQ ID NO: 51 - protein, 185 aa, Mus musculus
  Mmrn2 495-679
QRVNSDVDVAPEGHRDVMHTLEETQVSLDEQHQLDGSSLQALQSTVDAMSSAMDAYRGEGERARAERARIRSQLRALDHAVEALKTAANGTRKEIRLLHGSFTALLEDALRHQAVLAALFGEEMIDEMSEEAPRPLPLDYEQIRLALQDAASGLQEQAIGWEDLATRVEALEKAAGGFVEQHPQL

SEQ ID NO: 52 - protein, 198 aa, Danio rerio
  mmrn2a 615-812
IALGATVTHLEQTVADVMKTVNENILAQEMMANRHNRAWVPSVEDLKLGLLNVQNSLAFQQEKSRTLQHNVTQALASLLGSQQDIEMLQKQDRAKVEKIKQLEGTFNTLLQDATRHSDVLEILLGEEVLEFTKWSQQDKRRFSIPQLDERMKDMQKQINGHSLSLASMLNAATPQVTAADEPSLLADWASTGTKRKRE

SEQ ID NO: 53 - protein, 197 aa, Danio rerio
  mmrn2b 523-719
KVINSSLVRLEKEVDNVTQLARQNSLALEEADLERNEATEMEDLQHGLLNMKESLAFEQGRSRSLQDSILQLQDSLLDSKLEIQGLREQDGEQSAEIQGLSAAFSSLLNDAVRHSEVLEVLLGEEVLEFTHWSQSRQKDLSLPDLLQTIQMMQQKIETHDRSLTSLRRKHPDGDEMNSDDPVPYSEKPGMKQQMKGT

SEQ ID NO: 54 - protein, 203 aa, Gallus gallus
  Chicken MMRN2 526-728
QKIPSDANTLEEDLKNITYSLEGQQSKDVKQLESVFKDLLRNEIEELSSAFPFIHQSLNLRQEENRQLQLQVASFSEDIGLLKKKDEEIHRHIKYLNSSFGSLLEDAMRHEAALEALLGEEFMEVLFEEDPSTLISSVVQLQEFLKEISNKLQNQNVTLESLVKRLHHLERGQQNNHHVHASSKRPEQETQTLSMLDEANQLS

SEQ ID NO: 55 - protein, 152 aa, Homo sapiens
  CLEC14A 22-173
EHPTADRAGCSASGACYSLHHATMKRQAAEEACILRGGALSTVRAGAELRAVLALLRAGPGPGGGSKDLLFWVALERRRSHCTLENEPLRGFSWLSSDPGGLESDTLQWVEEPQRSCTARRCAVLQATGGVEPAGWKEMRCHLRANGYLCKY

SEQ ID NO: 56 - protein, 153 aa, Homo sapiens
  THBD 17-169
FPAPAEPQPGGSQCVEHDCFALYPGPATFLNASQICDGLRGHLMTVRSSVAADVISLLLNGDGGVGRRRLWIGLQLPPGCGDPKRLGPLRGFQWVTGDNNTSYSRWARLDLNGAPLCGPLCVAVSAAEATVPSEPIWEEQQCEVKADGFLCEF

SEQ ID NO: 57 - protein, 141 aa, Homo sapiens
  CD248 18-158
QDPWAAEPRAACGPSSCYALFPRRRTFLEAWRACRELGGDLATPRTPEEAQRVDSLVGAGPASRLLWIGLQRQARQCQLQRPLRGFTWTTGDQDTAFTNWAQPASGGPCPAQRCVALEASGEHRWLEGSCTLAVDGYLCQF

SEQ ID NO: 58 - protein, 151 aa, Homo sapiens
  CD93 24-174
ADTEAVVCVGTACYTAHSGKLSAAEAQNHCNQNGGNLATVKSKEEAQHVQRVLAQLLRREAALTARMSKFWIGLQREKGKCLDPSLPLKGFSWVGGGEDTPYSNWHKELRNSCISKRCVSLLLDLSQPLLPSRLPKWSEGPCGSPGSPGSN

SEQ ID NO: 59 - DNA, 2850 bp, Homo sapiens
  MMRN2 coding sequence
atgatcctgagcttgctgttcagccttgggggccccctgggctgggggctgctgggggcatgggcccaggcttccagtactagcctctctgatctgcagagctccaggacacctggggtctggaaggcagaggctgaggacaccggcaaggaccccgttggacgtaactggtgcccctacccaatgtccaagctggtcaccttactagctctttgcaaaacagagaaattcctcatccactcgcagcagccgtgtccgcagggagctccagactgccagaaagtcaaagtcatgtaccgcatggcccacaagccagtgtaccaggtcaagcagaaggtgctgacctctttggcctggaggtgctgccctggctacacgggccccaactgcgagcaccacgattccatggcaatccctgagcctgcagatcctggtgacagccaccaggaacctcaggatggaccagtcagcttcaaacctggccaccttgctgcagtgatcaatgaggttgaggtgcaacaggaacagcaggaacatctgctgggagatctccagaatgatgtgcaccgggtggcagacagcctgccaggcctgtggaaagccctgcctggtaacctcacagctgcagtgatggaagcaaatcaaacagggcacgagttccctgatagatccttggagcaggtgctgctaccccacgtggacaccttcctacaagtgcatttcagccccatctggaggagctttaaccaaagcctgcacagccttacccaggccataagaaacctgtctcttgacgtggaggccaaccgccaggccatctccagagtccaggacagtgccgtggccagggctgacttccaggagcttggtgccaaatttgaggccaaggtccaggagaacactcagagagtgggtcagctgcgacaggacgtggaggaccgcctgcacgcccagcactttaccctgcaccgctcgatctcagagctccaagccgatgtggacaccaaattgaagaggctgcacaaggctcaggaggccccagggaccaatggcagtctggtgttggcaacgcctggggctggggcaaggcctgagccggacagcctgcaggccaggctgggccagctgcagaggaacctctcagagctgcacatgaccacggcccgcagggaggaggagttgcagtacaccctggaggacatgagggccaccctgacccggcacgtggatgagatcaaggaactgtactccgaatcggacgagactttcgatcagattagcaaggtggagcggcaggtggaggagctgcaggtgaaccacacggcgctccgtgagctgcgcgtgatcctgatggagaagtctctgatcatggaggagaacaaggaggaggtggagcggcagctcctggagctcaacctcacgctgcagcacctgcagggtggccatgccgacctcatcaagtacgtgaaggactgcaattgccagaagctctatttagacctggacgtcatccgggagggccagagggacgccacgcgtgccctggaggagacccaggtgagcctggacgagcggcggcagctggacggctcctccctgcaggccctgcagaacgccgtggacgccgtgtcgctggccgtggacgcgcacaaagcggagggcgagcgggcgcgggcggccacgtcgcggctccggagccaagtgcaggcgctggatgacgaggtgggcgcgctgaaggcggccgcggccgaggcccgccacgaggtgcgccagctgcacagcgccttcgccgccctgctggaggacgcgctgcggcacgaggcggtgctggccgcgctcttcggggaggaggtgctggaggagatgtctgagcagacgccgggaccgctgcccctgagctacgagcagatccgcgtggccctgcaggacgccgctagcgggctgcaggagcaggcgctcggctgggacgagctggccgcccgagtgacggccctggagcaggcctcggagcccccgcggccggcagagcacctggagcccagccacgacgcgggccgcgaggaggccgccaccaccgccctggccgggctggcgcgggagctccagagcctgagcaacgacgtcaagaatgtcgggcggtgctgcgaggctgaggccggggccggggccgcctccctcaacgcctcccttgacggcctccacaacgcactcttcgccactcagcgcagcttggagcagcaccagcggctcttccacagcctctttgggaacttccaagggctcatggaagccaacgtcagcctggacctggggaagctgcagaccatgctgagcaggaaagggaagaagcagcagaaagacctggaagctccccggaagagggacaagaaggaagcggagcctttggtggacatacgggtcacagggcctgtgccaggtgccttgggcgcggcgctctgggaggcaggatcccctgtggccttctatgccagcttttcagaagggacggctgccctgcagacagtgaagttcaacaccacatacatcaacattggcagcagctacttccctgaacatggctacttccgagcccctgagcgtggtgtctacctgtttgcagtgagcgttgaatttggcccagggccaggcaccgggcagctggtgtttggaggtcaccatcggactccagtctgtaccactgggcaggggagtggaagcacagcaacggtctttgccatggctgagctgcagaagggtgagcgagtatggtttgagttaacccagggatcaataacaaagagaagcctgtcgggcactgcatttgggggcttcctgatgtttaagacctga

SEQ ID NO: 60 - protein, 949 aa, Homo sapiens [sequence omitted]

SEQ ID NO: 61 - protein, 949 aa, Homo sapiens
  MMRN2 amino acid sequence
MILSLLFSLGGPLGWGLLGAWAQASSTSLSDLQSSRTPGVWKAEAEDTGKDPVGRNWCPYPMSKLVTLLALCKTEKFLIHSQQPCPQGAPDCQKVKVMYRMAHKPVYQVKQKVLTSLAWRCCPGYTGPNCEHHDSMAIPEPADPGDSHQEPQDGPVSFKPGHLAAVINEVEVQQEQQEHLLGDLQNDVHRVADSLPGLWKALPGNLTAAVMEANQTGHEFPDRSLEQVLLPHVDTFLQVHFSPIWRSFNQSLHSLTQAIRNLSLDVEANRQAISRVQDSAVARADFQELGAKFEAKVQENTQRVGQLRQDVEDRLHAQHFTLHRSISELQADVDTKLKRLHKAQEAPGTNGSLVLATPGAGARPEPDSLQARLGQLQRNLSELHMTTARREEELQYTLEDMRATLTRHVDEIKELYSESDETFDQISKVERQVEELQVNHTALRELRVILMEKSLIMEENKEEVERQLLELNLTLQHLQGGHADLIKYVKDCNCQKLYLDLDVIREGQRDATRALEETQVSLDERRQLDGSSLQALQNAVDAVSLAVDAHKAEGERARAATSRLRSQVQALDDEVGALKAAAAEARHEVRQLHSAFAALLEDALRHEAVLAALFGEEVLEEMSEQTPGPLPLSYEQIRVALQDAASGLQEQALGWDELAARVTALEQASEPPRPAEHLEPSHDAGREEAATTALAGLARELQSLSNDVKNVGRCCEAEAGAGAASLNASLHGLHNALFATQRSLEQHQRLFHSLFGNFQGLMEANVSLDLGKLQTMLSRKGKKQQKDLEAPRKRDKKEAEPLVDIRVTGPVPGALGAALWEAGSPVAFYASFSEGTAALQTVKFNTTYINIGSSYFPEHGYFRAPERGVYLFAVSVEFGPGPGTGQLVFGGHHRTPVCTTGQGSGSTATVFAMAELQKGERVWFELTQGSITKRSLSGTAFGGFLMFKT

SEQ ID NO: 62 - protein, 490 aa, Homo sapiens
  CLEC14A polypeptide sequence
MRPAFALCLLWQALWPGPGGGEHPTADRAGCSASGACYSLHHATMKRQAAEEACILRGGALSTVRAGAELRAVLALLRAGPGPGGGSKDLLFWVALERRRSHCTLENEPLRGFSWLSSDPGGLESDTLQWVEEPQRSCTARRCAVLQATGGVEPAGWKEMRCHLRANGYLCKYQFEVLCPAPRPGAASNLSYRAPFQLHSAALDFSPPGTEVSALCRGQLPISVTCIADEIGARWDKLSGDVLCPCPGRYLRAGKCAELPNCLDDLGGFACECATGFELGKDGRSCVTSGEGQPTLGGTGVPTRRPPATATSPVPQRTWPIRVDEKLGETPLVPEQDNSVTSIPEIPRWGSQSTMSTLQMSLQAESKATITPSGSVISKFNSTTSSATPQAFDSSSAVVFIFVSTAVVVLVILTMTVLGLVKLCFHESPSSQPRKESMGPPGLESDPEPAALGSSSAHCTNNGVKVGDCDLRDRAEGALLAESPLGSSDA

SEQ ID NO: 63 - DNA, 2256 bp, Homo sapiens
  CLEC14A cDNA sequence
ctcctcttgctctaagcagggtgtttgaccttctagtcgactgcgtcccctgtacccggcgccagctgtgttcctgaccccagaataactcagggctgcaccgggcctggcagcgctccgcacacatttcctgtcgcggcctaagggaaactgttggccgctgggcccgcggggggattcttggcagttggggggtccgtcgggagcgagggcggaggggaagggagggggaaccgggttggggaagccagctgtagagggcggtgaccgcgctccagacacagctctgcgtcctcgagcgggacagatccaagttgggagcagctctgcgtgcggggcctcagagaatgaggccggcgttcgccctgtgcctcctctggcaggcgctctggcccgggccgggcggcggcgaacaccccactgccgaccgtgctggctgctcggcctcgggggcctgctacagcctgcaccacgctaccatgaagcggcaggcggccgaggaggcctgcatcctgcgaggtggggcgctcagcaccgtgcgtgcgggcgccgagctgcgcgctgtgctcgcgctcctgcgggcaggcccagggcccggagggggctccaaagacctgctgttctgggtcgcactggagcgcaggcgttcccactgcaccctggagaacgagcctttgcggggtttctcctggctgtcctccgaccccggcggtctcgaaagcgacacgctgcagtgggtggaggagccccaacgctcctgcaccgcgcggagatgcgcggtactccaggccaccggtggggtcgagcccgcaggctggaaggagatgcgatgccacctgcgcgccaacggctacctgtgcaagtaccagtttgaggtcttgtgtcctgcgccgcgccccggggccgcctctaacttgagctatcgcgcgcccttccagctgcacagcgccgctctggacttcagtccacctgggaccgaggtgagtgcgctctgccggggacagctcccgatctcagttacttgcatcgcggacgaaatcggcgctcgctgggacaaactctcgggcgatgtgttgtgtccctgccccgggaggtacctccgtgctggcaaatgcgcagagctccctaactgcctagacgacttgggaggctttgcctgcgaatgtgctacgggcttcgagctggggaaggacggccgctcttgtgtgaccagtggggaaggacagccgacccttggggggaccggggtgcccaccaggcgcccgccggccactgcaaccagccccgtgccgcagagaacatggccaatcagggtcgacgagaagctgggagagacaccacttgtccctgaacaagacaattcagtaacatctattcctgagattcctcgatggggatcacagagcacgatgtctacccttcaaatgtcccttcaagccgagtcaaaggccactatcaccccatcagggagcgtgatttccaagtttaattctacgacttcctctgccactcctcaggctttcgactcctcctctgccgtggtcttcatatttgtgagcacagcagtagtagtgttggtgatcttgaccatgacagtactggggcttgtcaagctctgctttcacgaaagcccctcttcccagccaaggaaggagtctatgggcccgccgggcctggagagtgatcctgagcccgctgctttgggctccagttctgcacattgcacaaacaatggggtgaaagtcggggactgtgatctgcgggacagagcagagggtgccttgctggcggagtcccctcttggctctagtgatgcatagggaaacaggggacatgggcactcctgtgaacagtttttcacttttgatgaaacggggaaccaagaggaacttacttgtgtaactgacaatttctgcagaaatcccccttcctctaaattccctttactccactgaggagctaaatcagaactgcacactccttccctgatgatagaggaagtggaagtgcctttaggatggtgatactgggggaccgggtagtgctggggagagatattttcttatgtttattcggagaatttggagaagtgattgaacttttcaagacattggaaacaaatagaacacaatataatttacattaaaaaataatttctaccaaaatggaaaggaaatgttctatgttgttcaggctaggagtatattggttcgaaatcccagggaaaaaaataaaaataaaaaattaaaggattgt

SEQ ID NO: 64 - DNA, 1473 bp, Homo sapiens
  CLEC14A coding sequence
atgaggccggcgttcgccctgtgcctcctctggcaggcgctctggcccgggccgggcggcggcgaacaccccactgccgaccgtgctggctgctcggcctcgggggcctgctacagcctgcaccacgctaccatgaagcggcaggcggccgaggaggcctgcatcctgcgaggtggggcgctcagcaccgtgcgtgcgggcgccgagctgcgcgctgtgctcgcgctcctgcgggcaggcccagggcccggagggggctccaaagacctgctgttctgggtcgcactggagcgcaggcgttcccactgcaccctggagaacgagcctttgcggggtttctcctggctgtcctccgaccccggcggtctcgaaagcgacacgctgcagtgggtggaggagccccaacgctcctgcaccgcgcggagatgcgcggtactccaggccaccggtggggtcgagcccgcaggctggaaggagatgcgatgccacctgcgcgccaacggctacctgtgcaagtaccagtttgaggtcttgtgtcctgcgccgcgccccggggccgcctctaacttgagctatcgcgcgcccttccagctgcacagcgccgctctggacttcagtccacctgggaccgaggtgagtgcgctctgccggggacagctcccgatctcagttacttgcatcgcggacgaaatcggcgctcgctgggacaaactctcgggcgatgtgttgtgtccctgccccgggaggtacctccgtgctggcaaatgcgcagagctccctaactgcctagacgacttgggaggctttgcctgcgaatgtgctacgggcttcgagctggggaaggacggccgctcttgtgtgaccagtggggaaggacagccgacccttggggggaccggggtgcccaccaggcgcccgccggccactgcaaccagccccgtgccgcagagaacatggccaatcagggtcgacgagaagctgggagagacaccacttgtccctgaacaagacaattcagtaacatctattcctgagattcctcgatggggatcacagagcacgatgtctacccttcaaatgtcccttcaagccgagtcaaaggccactatcaccccatcagggagcgtgatttccaagtttaattctacgacttcctctgccactcctcaggctttcgactcctcctctgccgtggtcttcatatttgtgagcacagcagtagtagtgttggtgatcttgaccatgacagtactggggcttgtcaagctctgctttcacgaaagcccctcttcccagccaaggaaggagtctatgggcccgccgggcctggagagtgatcctgagcccgctgctttgggctccagttctgcacattgcacaaacaatggggtgaaagtcggggactgtgatctgcgggacagagcagagggtgccttgctggcggagtcccctcttggctctagtgatgcatag

SEQ ID NO: 65 - protein, 652 aa, Homo sapiens
  CD93 polypeptide sequence
MATSMGLLLLLLLLLTQPGAGTGADTEAVVCVGTACYTAHSGKLSAAEAQNHCNQNGGNLATVKSKEEAQHVQRVLAQLLRREAALTARMSKFWIGLQREKGKCLDPSLPLKGFSWVGGGEDTPYSNWHKELRNSCISKRCVSLLLDLSQPLLPSRLPKWSEGPCGSPGSPGSNIEGFVCKFSFKGMCRPLALGGPGQVTYTTPFQTTSSSLEAVPFASAANVACGEGDKDETQSHYFLCKEKAPDVFDWGSSGPLCVSPKYGCNFNNGGCHQDCFEGGDGSFLCGCRPGFRLLDDLVTCASRNPCSSSPCRGGATCVLGPHGKNYTCRCPQGYQLDSSQLDCVDVDECQDSPCAQECVNTPGGFRCECWVGYEPGGPGEGACQDVDECALGRSPCAQGCTNTDGSFHCSCEEGYVLAGEDGTQCQDVDECVGPGGPLCDSLCFNTQGSFHCGCLPGWVLAPNGVSCTMGPVSLGPPSGPPDEEDKGEKEGSTVPRAATASPTRGPEGTPKATPTTSRPSLSSDAPITSAPLKMLAPSGSPGVWREPSIHHATAASGPQEPAGGDSSVATQNNDGTDGQKLLLFYILGTVVAILLLLALALGLLVYRKRRAKREEKKEKKPQNAADSYSWVPERAESRAMENQYSPTPGTDC

SEQ ID NO: 66 - DNA, 6701 bp, Homo sapiens
  CD93 cDNA sequence
aaagccctcagcctttgtgtccttctctgcgccggagtggctgcagctcacccctcagctccccttggggcccagctgggagccgagatagaagctcctgtcgccgctgggcttctcgcctcccgcagagggccacacagagaccgggatggccacctccatgggcctgctgctgctgctgctgctgctcctgacccagcccggggcggggacgggagctgacacggaggcggtggtctgcgtggggaccgcctgctacacggcccactcgggcaagctgagcgctgccgaggcccagaaccactgcaaccagaacgggggcaacctggccactgtgaagagcaaggaggaggcccagcacgtccagcgagtactggcccagctcctgaggcgggaggcagccctgacggcgaggatgagcaagttctggattgggctccagcgagagaagggcaagtgcctggaccctagtctgccgctgaagggcttcagctgggtgggcgggggggaggacacgccttactctaactggcacaaggagctccggaactcgtgcatctccaagcgctgtgtgtctctgctgctggacctgtcccagccgctccttcccagccgcctccccaagtggtctgagggcccctgtgggagcccaggctcccccggaagtaacattgagggcttcgtgtgcaagttcagcttcaaaggcatgtgccggcctctggccctggggggcccaggtcaggtgacctacaccacccccttccagaccaccagttcctccttggaggctgtgccctttgcctctgcggccaatgtagcctgtggggaaggtgacaaggacgagactcagagtcattatttcctgtgcaaggagaaggcccccgatgtgttcgactggggcagctcgggccccctctgtgtcagccccaagtatggctgcaacttcaacaatgggggctgccaccaggactgctttgaagggggggatggctccttcctctgcggctgccgaccaggattccggctgctggatgacctggtgacctgtgcctctcgaaacccttgcagctccagcccatgtcgtgggggggccacgtgcgtcctgggaccccatgggaaaaactacacgtgccgctgcccccaagggtaccagctggactcgagtcagctggactgtgtggacgtggatgaatgccaggactccccctgtgcccaggagtgtgtcaacacccctgggggcttccgctgcgaatgctgggttggctatgagccgggcggtcctggagagggggcctgtcaggatgtggatgagtgtgctctgggtcgctcgccttgcgcccagggctgcaccaacacagatggctcatttcactgctcctgtgaggagggctacgtcctggccggggaggacgggactcagtgccaggacgtggatgagtgtgtgggcccggggggccccctctgcgacagcttgtgcttcaacacacaagggtccttccactgtggctgcctgccaggctgggtgctggccccaaatggggtctcttgcaccatggggcctgtgtctctgggaccaccatctgggccccccgatgaggaggacaaaggagagaaagaagggagcaccgtgccccgtgctgcaacagccagtcccacaaggggccccgagggcacccccaaggctacacccaccacaagtagaccttcgctgtcatctgacgcccccatcacatctgccccactcaagatgctggcccccagtgggtccccaggcgtctggagggagcccagcatccatcacgccacagctgcctctggcccccaggagcctgcaggtggggactcctccgtggccacacaaaacaacgatggcactgacgggcaaaagctgcttttattctacatcctaggcaccgtggtggccatcctactcctgctggccctggctctggggctactggtctatcgcaagcggagagcgaagagggaggagaagaaggagaagaagccccagaatgcggcagacagttactcctgggttccagagcgagctgagagcagggccatggagaaccagtacagtccgacacctgggacagactgctgaaagtgaggtggccctagagacactagagtcaccagccaccatcctcagagctttgaactccccattccaaaggggcacccacatttttttgaaagactggactggaatcttagcaaacaattgtaagtctcctccttaaaggccccttggaacatgcaggtattttctacgggtgtttgatgttcctgaagtggaagctgtgtgttggcgtgccacggtggggatttcgtgactctataatgattgttactccccctcccttttcaaattccaatgtgaccaattccggatcagggtgtgaggaggccggggctaaggggctcccctgaatatcttctctgctcacttccaccatctaagaggaaaaggtgagttgctcatgctgattaggattgaaatgatttgtttctcttcctaggatgaaaactaaatcaattaattattcaattaggtaagaagatctggttttttggtcaaagggaacatgttcggactggaaacatttctttacatttgcattcctccatttcgccagcacaagtcttgctaaatgtgatactgttgacatcctccagaatggccagaagtgcaattaacctcttaggtggcaaggaggcaggaagtgcctctttagttcttacatttctaatagccttgggtttatttgcaaaggaagcttgaaaaatatgagaaaagttgcttgaagtgcattacaggtgtttgtgaagtcacataatctacggggctagggcgagagaggccagggatttgttcacagatacttgaattaattcatccaaatgtactgaggttaccacacacttgactacggatgtgatcaacactaacaaggaaacaaattcaaggacaacctgtctttgagccagggcaggcctcagacaccctgcctgtggccccgcctccacttcatcctgcccggaatgccagtgctccgagctcagacagaggaagccctgcagaaagttccatcaggctgtttcctaaaggatgtgtgaacgggagatgatgcactgtgttttgaaagttgtcattttaaagcattttagcacagttcatagtccacagttgatgcagcatcctgagattttaaatcctgaagtgtgggtggcgcacacaccaagtagggagctagtcaggcagtttgcttaaggaacttttgttctctgtctcttttccttaaaattgggggtaaggagggaaggaagagggaaagagatgactaactaaaatcatttttacagcaaaaactgctcaaagccatttaaattatatcctcattttaaaagttacatttgcaaatatttctccctatgataatgtagtcgatagtgtgcactctttctctctctctctctctctcacacacacacacacacacacacacacacacacagagacacggcaccattctgcctggggcactggaacacattcctgggggtcaccgatggtcagagtcactagaagttacctgagtatctctgggaggcctcatgtctcctgtgggctttttaccaccactgtgcaggagaacagacagaggaaatgtgtctccctccaaggccccaaagcctcagagaaagggtgtttctggttttgccttagcaatgcatcggtctctgaggtgacactctggagtggttgaagggccacaaggtgcagggttaatactcttgccagttttgaaatatagatgctatggttcagattgtttttaatagaaaactaaaggggcaggggaagtgaaaggaaagatggaggttttgtgcggctcgatggggcatttggaacttctttttaaagtcatctcatggtctccagttttcagttggaactctggtgtttaacacttaagggagacaaaggctgtgtccatttggcaaaacttccttggccacgagactctaggtgatgtgtgaagctgggcagtctgtggtgtggagagcagccatctgtctggccattcagaggattctaaagacatggctggatgcgctgctgaccaacatcagcacttaaataaatgcaaatgcaacatttctccctctgggccttgaaaatccttgcccttatcatttggggtgaaggagacatttctgtccttggcttcccacagccccaacgcagtctgtgtatgattcctgggatccaacgagccctcctattttcacagtgttctgattgctctcacagcccaggcccatcgtctgttctctgaatgcagccctgttctcaacaacagggaggtcatggaacccctctgtggaacccacaaggggagaaatgggtgataaagaatccagttcctcaaaaccttccctggcaggctgggtccctctcctgctgggtggtgctttctcttgcacaccactcccaccacggggggagagccagcaacccaaccagacagctcaggttgtgcatctgatggaaaccactgggctcaaacacgtgctttattctcctgtttatttttgctgttactttgaagcatggaaattcttgtttgggggatcttggggctacagtagtgggtaaacaaatgcccaccggccaagaggccattaacaaatcgtccttgtcctgaggggccccagcttgctcgggcgtggcacagtggggaatccaagggtcacagtatggggagaggtgcaccctgccacctgctaacttctcgctagacacagtgtttctgcccaggtgacctgttcagcagcagaacaagccagggccatggggacgggggaagttttcacttggagatggacaccaagacaatgaagatttgttgtccaaataggtcaataattctgggagactcttggaaaaaactgaatatattcaggaccaactctctccctcccctcatcccacatctcaaagcagacaatgtaaagagagaacatctcacacacccagctcgccatgcctactcattcctgaatttcaggtgccatcactgctctttctttcttctttgtcatttgagaaaggatgcaggaggacaattcccacagataatctgaggaatgcagaaaaaccagggcaggacagttatcgacaatgcattagaacttggtgagcatcctctgtagagggactccacccctgctcaacagcttggcttccaggcaagaccaaccacatctggtctctgccttcggtggcccacacacctaagcgtcatcgtcattgccatagcatcatgatgcaacacatctacgtgtagcactacgacgttatgtttgggtaatgtggggatgaactgcatgaggctctgattaaggatgtggggaagtgggctgcggtcactgtcggccttgcaaggccacctggaggcctgtctgttagccagtggtggaggagcaaggcttcaggaagggccagccacatgccatcttccctgcgatcaggcaaaaaagtggaattaaaaagtcaaacctttatatgcatgtgttatgtccattttgcaggatgaactgagtttaaaagaatttttttttctcttcaagttgctttgtcttttccatcctcatcacaagcccttgtttgagtgtcttatccctgagcaatctttcgatggatggagatgatcattaggtacttttgtttcaacctttattcctgtaaatatttctgtgaaaactaggagaacagagatgagatttgacaaaaaaaaattgaattaaaaataacacagtctttttaaaactaacataggaaagcctttcctattatttctcttcttagcttctccattgtctaaatcaggaaaacaggaaaacacagctttctagcagctgcaaaatggtttaatgccccctacatatttccatcaccttgaacaatagctttagcttgggaatctgagatatgatcccagaaaacatctgtctctacttcggctgcaaaacccatggtttaaatctatatggtttgtgcattttctcaactaaaaatagagatgataatccgaattctccatatattcactaatcaaagacactattttcatactagattcctgagacaaatactcactgaagggcttgtttaaaaataaattgtgttttggtctgttcttgtagataatgcccttctattttaggtagaagctctggaatccctttattgtgctgttgctcttatctgcaaggtggcaagcagttcttttcagcagattttgcccactattcctctgagctgaagttctttgcatagatttggcttaagcttgaattagatccctgcaaaggcttgctctgtgatgtcagatgtaattgtaaatgtcagtaatcacttcatgaatgctaaatgagaatgtaagtatttttaaatgtgtgtatttcaaatttgtttgactaattctggaattacaagatttctatgcaggatttaccttcatcctgtgcatgtttcccaaactgtgaggagggaaggctcagagatcgagcttctcctctgagttctaacaaaatggtgctttgagggtcagcctttaggaaggtgcagctttgttgtcctttgagctttctgttatgtgcctatcctaataaactcttaaacacattga

SEQ ID NO: 67 - DNA, 1959 bp, Homo sapiens
  CD93 coding sequence
atggccacctccatgggcctgctgctgctgctgctgctgctcctgacccagcccggggcggggacgggagctgacacggaggcggtggtctgcgtggggaccgcctgctacacggcccactcgggcaagctgagcgctgccgaggcccagaaccactgcaaccagaacgggggcaacctggccactgtgaagagcaaggaggaggcccagcacgtccagcgagtactggcccagctcctgaggcgggaggcagccctgacggcgaggatgagcaagttctggattgggctccagcgagagaagggcaagtgcctggaccctagtctgccgctgaagggcttcagctgggtgggcgggggggaggacacgccttactctaactggcacaaggagctccggaactcgtgcatctccaagcgctgtgtgtctctgctgctggacctgtcccagccgctccttcccagccgcctccccaagtggtctgagggcccctgtgggagcccaggctcccccggaagtaacattgagggcttcgtgtgcaagttcagcttcaaaggcatgtgccggcctctggccctggggggcccaggtcaggtgacctacaccacccccttccagaccaccagttcctccttggaggctgtgccctttgcctctgcggccaatgtagcctgtggggaaggtgacaaggacgagactcagagtcattatttcctgtgcaaggagaaggcccccgatgtgttcgactggggcagctcgggccccctctgtgtcagccccaagtatggctgcaacttcaacaatgggggctgccaccaggactgctttgaagggggggatggctccttcctctgcggctgccgaccaggattccggctgctggatgacctggtgacctgtgcctctcgaaacccttgcagctccagcccatgtcgtgggggggccacgtgcgtcctgggaccccatgggaaaaactacacgtgccgctgcccccaagggtaccagctggactcgagtcagctggactgtgtggacgtggatgaatgccaggactccccctgtgcccaggagtgtgtcaacacccctgggggcttccgctgcgaatgctgggttggctatgagccgggcggtcctggagagggggcctgtcaggatgtggatgagtgtgctctgggtcgctcgccttgcgcccagggctgcaccaacacagatggctcatttcactgctcctgtgaggagggctacgtcctggccggggaggacgggactcagtgccaggacgtggatgagtgtgtgggcccggggggccccctctgcgacagcttgtgcttcaacacacaagggtccttccactgtggctgcctgccaggctgggtgctggccccaaatggggtctcttgcaccatggggcctgtgtctctgggaccaccatctgggccccccgatgaggaggacaaaggagagaaagaagggagcaccgtgccccgtgctgcaacagccagtcccacaaggggccccgagggcacccccaaggctacacccaccacaagtagaccttcgctgtcatctgacgcccccatcacatctgccccactcaagatgctggcccccagtgggtccccaggcgtctggagggagcccagcatccatcacgccacagctgcctctggcccccaggagcctgcaggtggggactcctccgtggccacacaaaacaacgatggcactgacgggcaaaagctgcttttattctacatcctaggcaccgtggtggccatcctactcctgctggccctggctctggggctactggtctatcgcaagcggagagcgaagagggaggagaagaaggagaagaagccccagaatgcggcagacagttactcctgggttccagagcgagctgagagcagggccatggagaaccagtacagtccgacacctgggacagactgctga

(19) United States
(12) Patent Application Publication
(10) Pub. No.: US 2019/0185532 A1
(43) Pub. Date: Jun. 20, 2019

(54) INHIBITORS

(71) Applicant: THE UNIVERSITY OF BIRMINGHAM, Birmingham (GB)

(72) Inventors: Roy Bicknell, Edgbaston Birmingham (GB); Kabir Ali Khan, Toronto, Ontario (CA)

(21) Appl. No.: 16/320,522

(22) PCT Filed: Jul. 24, 2017
(86) PCT No.: PCT/GB2017/052154

§ 371 Date: Jan. 25, 2019

(30) Foreign Application Priority Data
Jul. 25, 2016 (GB) 1612860.5

(51) Int. Cl. C07K 14/47; C07K 16/30; C07K 14/725; C07K 14/705; A61K 45/06; A61K 38/17; A61K 39/395; A61P 35/00; A61K 51/08; G01N 33/574

(52) U.S. Cl. CPC C07K 14/4748; C07K 2319/02; C07K 14/7051; C07K 14/70521; A61K 45/06; A61K 38/1774; A61K 39/39558; A61P 35/00; A61K 51/08; C07K 14/7056; A61K 38/178; C07K 14/70596; A61K 38/177; G01N 33/57492; C07K 2317/34; C07K 2317/76; C07K 2319/03; C07K 16/30

(57) ABSTRACT

The invention provides a portion of multimerin 2 (MMRN2) or a variant thereof, that inhibits the interaction between CLEC14A and MMRN2, in addition to a portion of MMRN2 or a variant thereof, that inhibits the interaction between CD93 and MMRN2. The invention provides compounds comprising said portions and either a cytotoxic moiety or a detectable moiety.

The present invention relates generally to endothelium specific genes and polypeptides, inhibitors of these endothelium specific genes/polypeptides for inhibiting angiogenesis and combating other diseases, and the use of agents that bind to these polypeptides for imaging and targeting neovasculature. In particular, the present invention relates to portions of MMRN2, and specifically to portions of MMRN2 that inhibit the interaction between MMRN2 and CLEC14A and/or that inhibit the interaction between MMRN2 and CD93. The invention also relates to antibodies that bind to said portions.

The listing or discussion of an apparently prior-published document in this specification should not necessarily be taken as an acknowledgement that the document is part of the state of the art or is common general knowledge.

Endothelial cells form a single cell layer that lines all blood vessels and regulates exchanges between the blood stream and the surrounding tissues. New blood vessels develop from the walls of existing small vessels by the outgrowth of endothelial cells in the process called angiogenesis. Endothelial cells even have the capacity to form hollow capillary tubes when isolated in culture. Once the vascular system is fully developed, endothelial cells of blood vessels normally remain quiescent with no new vessel formation, with the exception of the formation of new blood vessels in natural wound healing. However, some tumours attract a new blood supply by secreting factors that stimulate nearby endothelial cells to construct new capillary sprouts. Angiogenesis plays a major role in the progression of solid tumours and is widely recognised as a rate-limiting process in the growth of solid tumours. Tumours that fail to attract a blood supply are severely limited in their growth. Thus the ability to inhibit inappropriate or undesirable angiogenesis may be useful in the treatment of solid tumours.

The development of new blood vessels is essential for both local tumour progression and the development of distant metastases. Indeed, the growth and survival of tumours is dependent on their ability to obtain a blood supply and damage inflicted on the tumour endothelium has been shown to effectively eradicate tumours (Burrows et a/ (1993) “Eradication of large solid tumors in mice with an immunotoxin directed against tumor vasculature.” Proc Natl Acad Sci USA, 90(19): 8996-9000). Tumour angiogenesis involves the degradation of the basement membrane by activated tissue or circulating endothelial precursors, proliferation and migration of endothelial cells, interaction with the extracellular matrix, morphological differentiation, cell adherence and vascular tube formation. Inhibition of tumour angiogenesis is thus a target for anti-tumour therapies, employing either angiogenesis inhibitors alone or in combination with standard cancer treatments. However, targeting anti-tumour agents to the site of angiogenesis depends upon the identification of specific markers of tumour angiogenesis. It is now accepted that the growth of solid tumours is dependent on their capacity to acquire a blood supply, and much effort has been directed towards the development of anti-angiogenic agents that disrupt this process. It has also become apparent that targeted destruction of the established tumour vasculature is another avenue for exciting therapeutic opportunities, and the discovery of widely expressed tumour endothelial markers promises much clinical benefit (Neri & Bicknell (2005) “Tumour vascular targeting.” Nat Rev Cancer 5(6): 436-446).

The inventors have previously identified CLEC14A as a tumour endothelial marker (WO 2011/027132). CLEC14A is a single-pass transmembrane glycoprotein that belongs to the vascular restricted C-type lectin family 14, whose other members include CD248/TEM1/Endosialin, Thrombomodulin and CD93. Available data on CLEC14A suggests that manipulation of CLEC14A levels or function blocking antibodies will regulate endothelial migration (WO2011/027132).

The inventors have also previously demonstrated that the interaction between CLEC14A and multimerin 2 (MMRN2) plays an important role in angiogenesis (PCT/GB2016/050134). MMRN2 is an endothelial specific marker of the emilin family and a component of the extracellular matrix. MMRN2 is an extracellular interacting protein for CLEC14A and was found to be co-expressed with CLEC14A in the tumour vasculature.

The inventors have now shown that CD93 as well as CLEC14A can directly bind to MMRN2. Binding of CLEC14A and CD93 has been mapped to a non-glycosylated coiled-coil region of MMRN2, and a CLEC14A and CD93 binding portion of MMRN2 has been shown both to disrupt angiogenesis in an in vitro assay and increase adherence of HUVEC cells. Further, the portion of MMRN2 expressed by Lewis lung carcinoma cells inhibited their growth in vivo. Taken together, the inventors' findings propose previously unknown protein interactions in the endothelium, and new therapeutic avenues in inhibiting angiogenesis and combating cancer.

Accordingly, a first aspect of the invention provides a portion of multimerin 2 (MMRN2) or a variant thereof, which portion inhibits the interaction between CLEC14A and MMRN2.

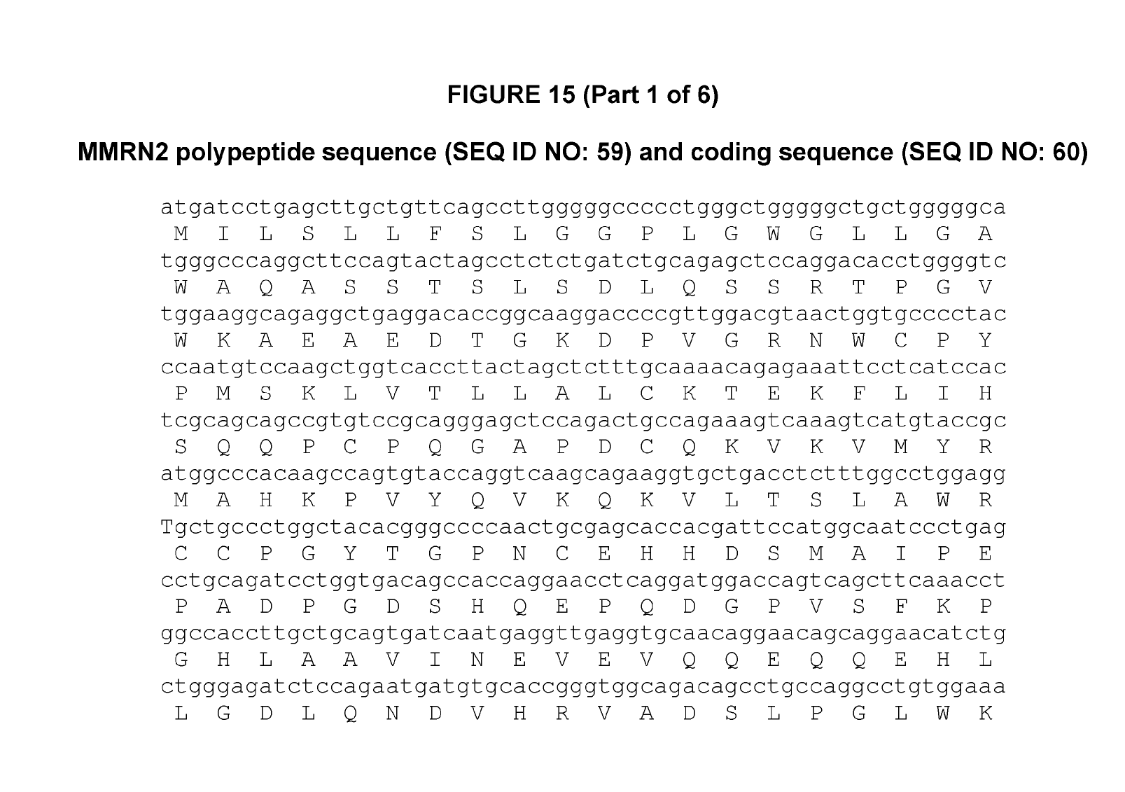

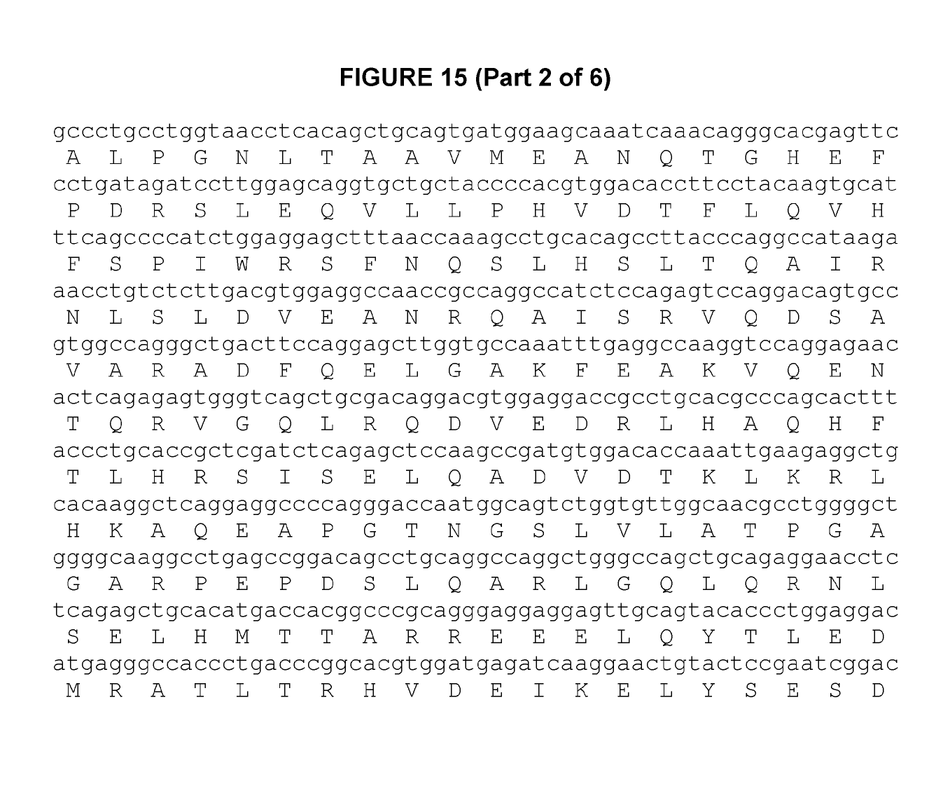

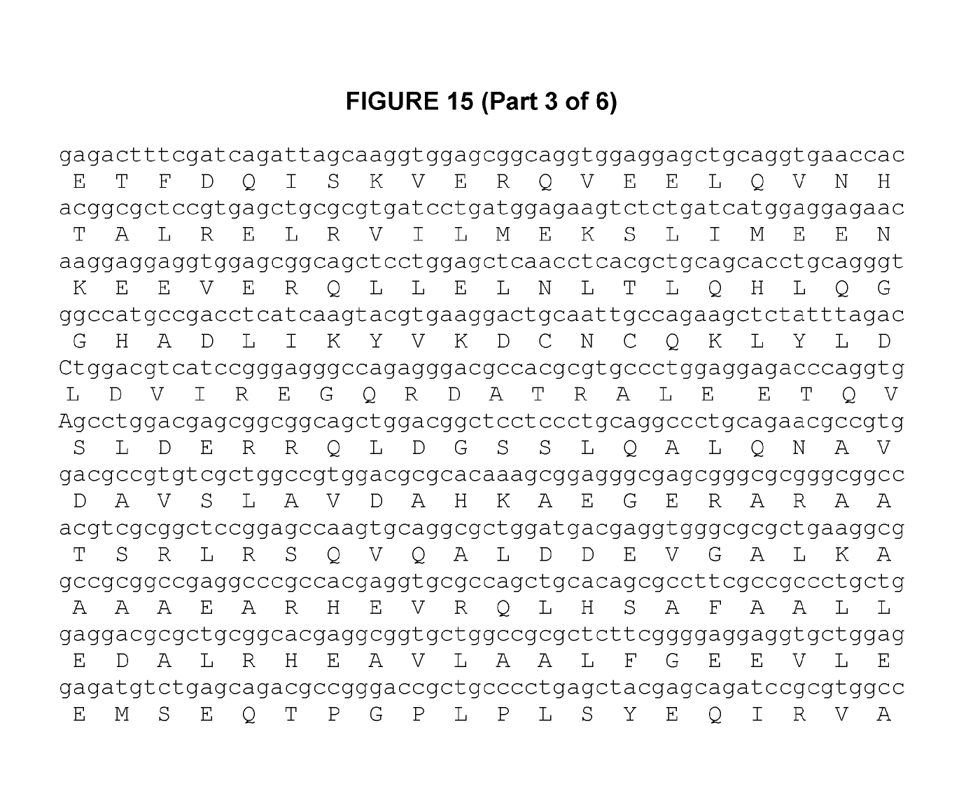

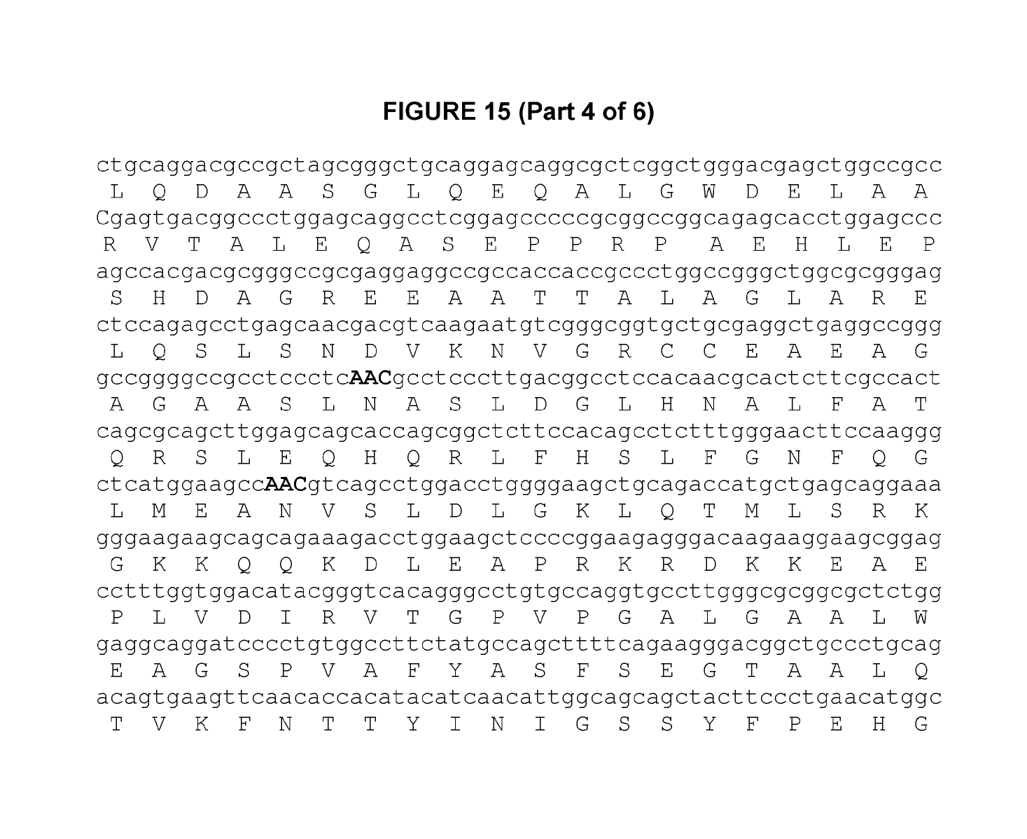

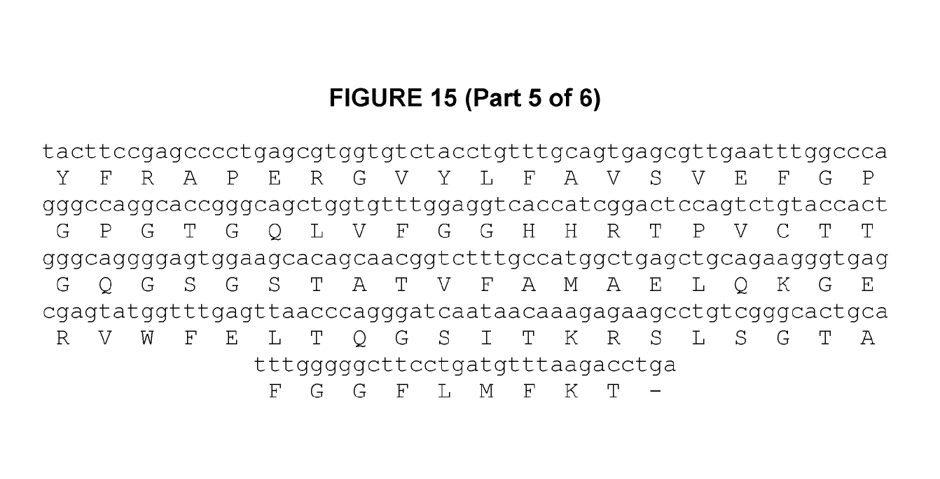

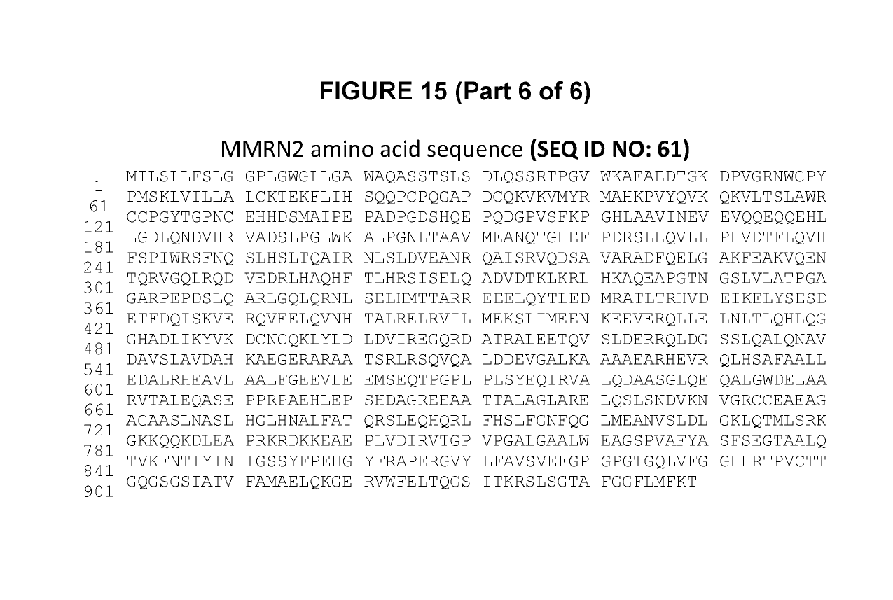

The gene MMRN2 is located at 10q23.2 and encodes a 888 amino acid residue polypeptide. By the MMRN2 polypeptide we include the meaning of a gene product of human MMRN2, including naturally occurring variants thereof. Human MMRN2 polypeptide includes the amino acid sequence found in Genbank Accession No XP_006718033 and naturally occurring variants thereof. The MMRN2 polypeptide sequence from XP_006718033 is shown in FIG. 15. Also included are MMRN2 orthologues found in other species, such as in horse, bull, chimp, chicken, zebrafish, dog, pig, cow, sheep, rat, mouse, guinea pig or a primate.

By “natural variants” we include, for example, allelic variants. Typically, these will vary from the given sequence by only one or two or three, and typically no more than 10 or 20 amino acid residues. Typically, the variants have conservative substitutions.

A cDNA sequence corresponding to a human MMRN2 mRNA is found in Genbank Accession No NM_024756.2, and the coding region is also shown in FIG. 15.

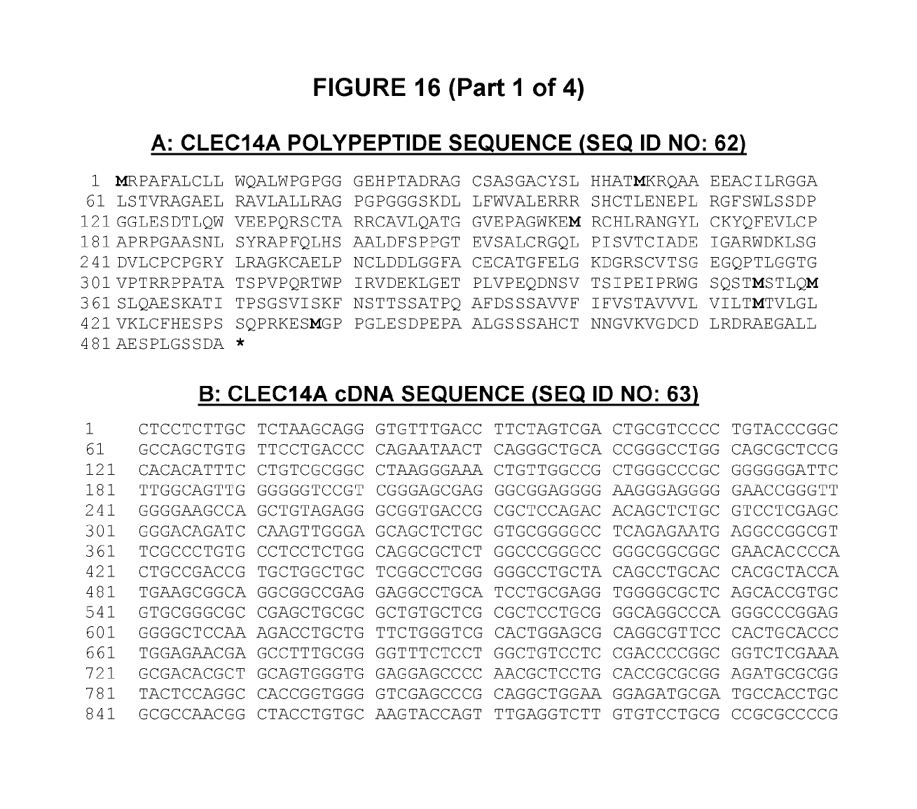

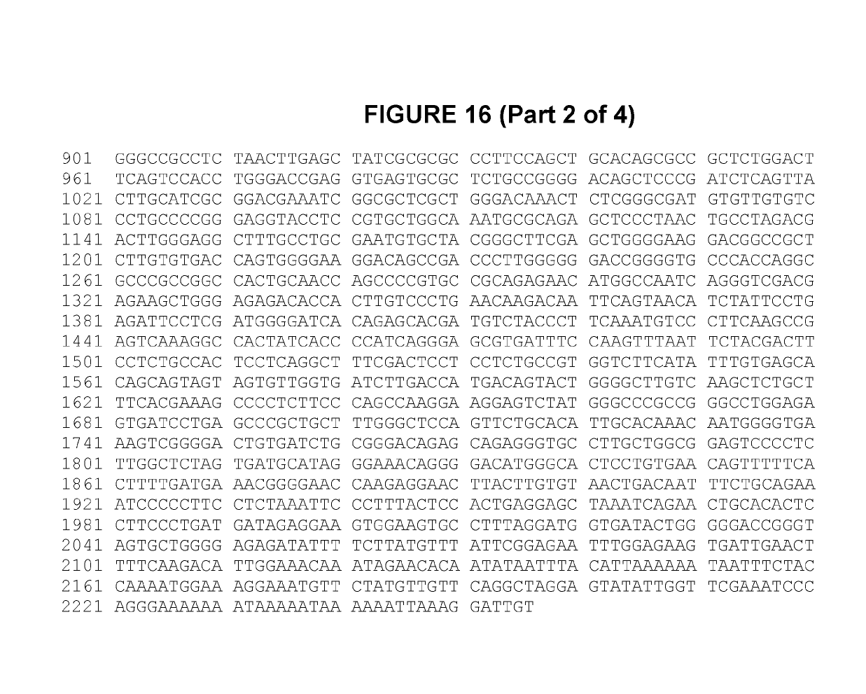

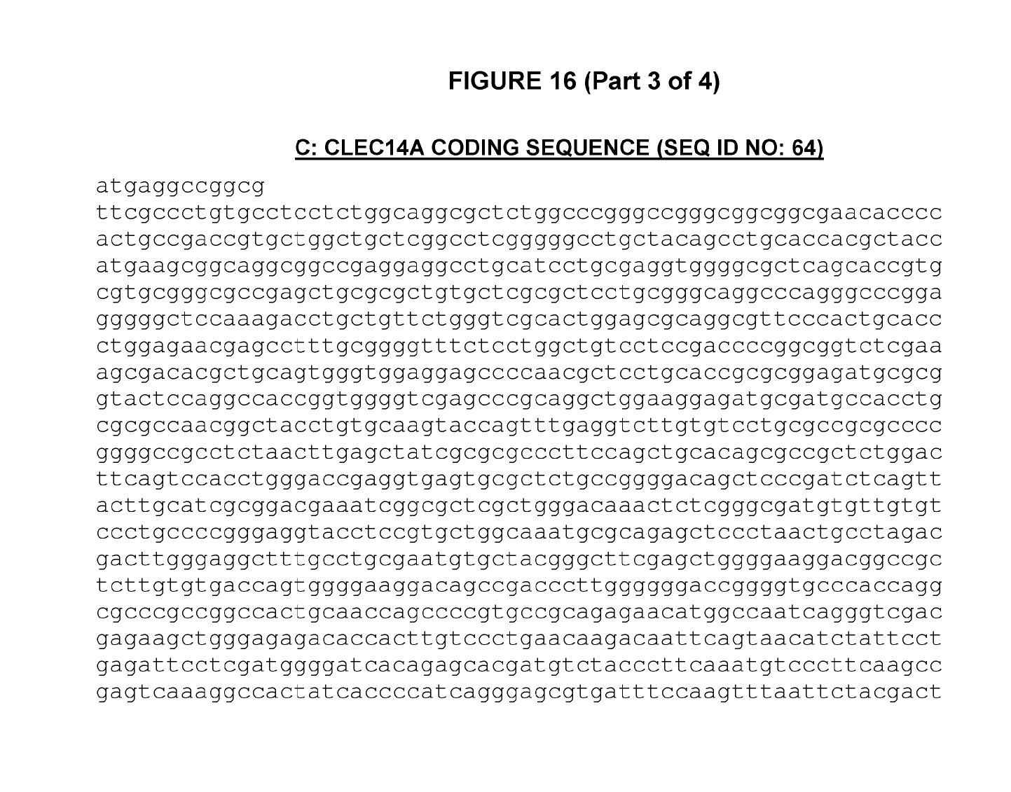

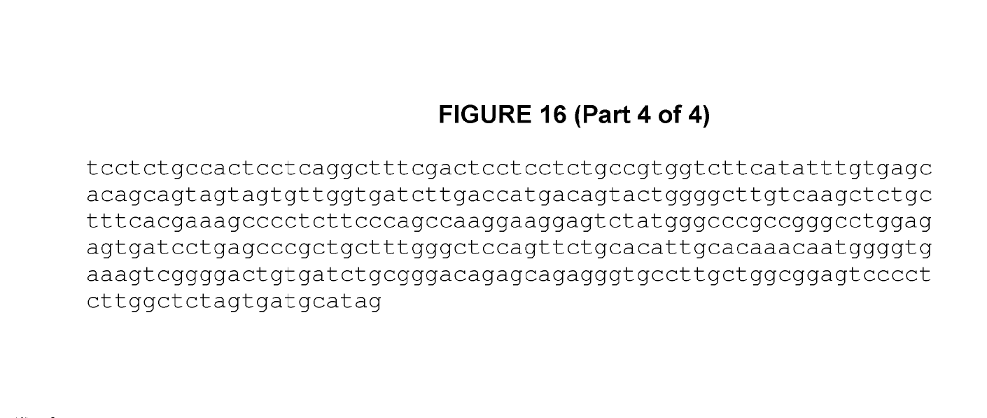

The gene CLEC14A (C-type lectin domain family 14, member A), which is located at 14q21.1, was previously known as C14orf27, CE.G.1 and E.G.FR5. CLEC14A encodes a 490 amino acid residue polypeptide with a predicted MW of 51 kDa. By the CLEC14A polypeptide we include the meaning of a gene product of human CLEC14A, including naturally occurring variants thereof. Human CLEC14A polypeptide includes the amino acid sequence found in Genbank Accession No NP_778230 and naturally occurring variants thereof. The CLEC14A polypeptide sequence from NP_778230 is shown in FIG. 16. Also included are CLEC14A orthologues found in other species, such as in horse, dog, pig, cow, sheep, rat, mouse, guinea pig or a primate.

A cDNA sequence corresponding to a human CLEC14A mRNA is found in Genbank Accession No NM_175060 and shown in FIG. 16. The coding region of this cDNA from NM_175060 is from nucleotide 348 to nucleotide 1820, and this is also shown in FIG. 16.

CLEC14A is a type I transmembrane protein with a signal peptide at residues 1-21. The mature human polypeptide is 469 amino acids in length (amino acid residues 22-490), and contains a 375 residue extracellular region (residues 22-396), a transmembrane region (residues 397-425), and a cytoplasmic region (residues 426-490). The extracellular region contains a C-type lectin like domain (residues 32-173) and an E.G.F-like region (residues 245-287).

Portions of MMRN2

By a “portion of MMRN2 or a variant thereof”, we include the meaning of any portion of the full length native MMRN2 polypeptide or any portion of a variant of the full length native MMRN2 polypeptide, provided that the portion inhibits the interaction between MMRN2 and CLEC14A. In other words, the portion of MMRN2 is one that reduces the level of binding between CLEC14A and MMRN2, as compared to the level of binding between CLEC14A and MMRN2 in the absence of the portion of MMRN2.

Preferably, the portion is one that reduces the level of binding between CLEC14A and MMRN2 by at least 10%, 20%, 30%, 40% or 50%, and more preferably the portion is one that reduces the level of binding between CLEC14A and MMRN2 by at least 70%, 80%, 90%, 95% or 99%. Most preferably, the portion is one that reduces the level of binding between CLEC14A and MMRN2 to an undetectable level, or eliminates binding between CLEC14A and MMRN2. 0

Suitable methods for detecting and/or measuring (quantifying) the binding of CLEC14A to MMRN2 are well known to those skilled in the art. Examples of appropriate methods include pull-down assays, enzyme linked immunosorbent assays (ELISA), surface plasmon resonance assays, chip-based assays, immunocytofluorescence, yeast two-hybrid technology and phage display, which are common practice in the art and are described, for example, in Plant et al (1995) Analyt Biochem, 226(2), 342-348.and Sambrook et al (2001) Molecular Cloning A Laboratory Manual. Third Edition. Cold Spring Harbor Laboratory Press, Cold Spring Harbor, N.Y. Other methods of detecting binding between CLEC14A and MMRN2 include ultrafiltration with ion spray mass spectroscopy/HPLC methods or other physical and analytical methods. Fluorescence Energy Resonance Transfer (FRET) methods, for example, well known to those skilled in the art, may be used, in which binding of two fluorescent labelled entities (i.e. CLEC14A and MMRN2 or portions or variants thereof) may be measured by measuring the interaction of the fluorescent labels when in close proximity to each other.

It will be appreciated that the portion of MMRN2 may be a portion of a variant of native MMRN2. By a variant of native MMRN2, we include the meaning that the variant has at least 30% sequence identity to the wild type MMRN2 polypeptide (e.g. human MMRN2) over its entire length, for example at least 40%, 50%, 60%, 70% or 80% sequence identity. More preferably, the variant of MMRN2 has at least 85%, 90%, 95%, 96%, 97%, 98% or at least 99% sequence identity to the wild type MMRN2 polypeptide (e.g. human MMRN2) over its entire length. Generally, the variant of MMRN2 is one that retains one or more of the biological activities of the wild type MMRN2 polypeptide, such as binding to CLEC14A and/or CD93.

By “portion”, we include the meaning of any part of the full length MMRN2 polypeptide or variant thereof, and so the portion may be any portion of MMRN2 (or a variant thereof) that has less amino acids than the full length wild type polypeptide or a variant thereof. For example, human MMRN2 is 888 amino acids in length, and so a portion of human MMRN2 would have 887 amino acids or less, and so on.

A suitable portion is typically a contiguous portion of the MMRN2 polypeptide, or variant thereof, of at least 10 amino acids in length, such as at least, 20, 30, 40, 50, 60, 70, 80, 90, 100, 110, 120, 130, 140, 150, 160, 170, 180, 190 or 200 amino acids in length. Preferably, the portion is between 20 and 180 amino acids in length, such as between 25 and 179 amino acids in length, or 25 and 175 amino acids in length, or 25 and 100 amino acids in length. It will be appreciated that the contiguous portion of the MMRN2 may be larger, and so may be at least 250 amino acids in length, such as 300, 350, 400, 450, 500, 550, 600, 650, 700, 750, 800 or 850 amino acids in length. Generally, the portion is no more than 800 amino acids in length, such as no more than 700, 600, 500, or 400 amino acids. Preferably, the portion is no more than 300 amino acids in length, such as no more than 250, 240, 230, 220, 210, 200, 190, 180, 170, 160, 150, 140, 130, 120, 110, or 100, or 50 amino acids in length.

Generally, the portions of MMRN2 have at least 30% sequence identity to the amino acid sequence of the corresponding portion of the native MMRN2, for example at least 35%, 40%, 45%, 50%, 55%, 60%, 65%, 70%, 75%, or 80% sequence identity, and preferably, 85%, 90%, 95%, 96%, 97%, 98%, or 99% sequence identity, over the length of the portion. Thus, when the portion of MMRN2 is a portion of human MMRN2, the portion typically has at least 30% sequence identity to the amino acid sequence of the corresponding portion of the wild type human MMRN2, such as at least 35%, 40%, 45%, 50%, 55%, 60%, 65%, 70%, 75%, or 80% sequence identity, and preferably, 85%, 90%, 95%, 96%, 97%, 98%, or 99% sequence identity, over the length of the portion.

The percent sequence identity between two polypeptides may be determined using any suitable computer program, for example the GAP program of the University of Wisconsin Genetic Computing Group and it will be appreciated that percent identity is calculated in relation to polypeptides whose sequence has been aligned optimally. The alignment may alternatively be carried out using the Clustal W program Thompson et al., (1994) Nucleic Acids Res 22, 4673-80). The parameters used may be as follows: Fast pairwise alignment parameters: K-tuple(word) size; 1, window size; 5, gap penalty; 3, number of top diagonals; 5. Scoring method: x percent. Multiple alignment parameters: gap open penalty; 10, gap extension penalty; 0.05. Scoring matrix: BLOSUM.

For the avoidance of doubt, the invention does not include the full length wild type MMRN2 polypeptide or any naturally occurring variant thereof. Thus, the invention does not include the amino acid sequence of human MMRN2, or any other known MMRN2 orthologue or naturally occurring variant thereof.

In a preferred embodiment, the portion of MMRN2 of the first aspect of the invention is one that binds to CLEC14A, and more preferably is one that selectively binds to CLEC14A. By a portion of MMRN2 that selectively binds to CLEC14A, we include the meaning that the portion of MMRN2 binds CLEC14A with a greater affinity than for an irrelevant polypeptide such as human serum albumin. Preferably, the portion of MMRN2 binds CLEC14A with at least 5, or at least 10 or at least 50 times greater affinity than for the irrelevant polypeptide. More preferably, the portion of MMRN2 binds the CLEC14A with at least 100, or at least 1,000, or at least 10,000 times greater affinity than for the irrelevant polypeptide. Such binding may be determined by methods well known in the art, such as one of the Biacore® systems.

It is preferred that the portion of MMRN2 does not bind to thrombomodulin, or that the portion of MMRN2 binds CLEC14A with a greater affinity than for thrombomodulin. Preferably, the portion of MMRN2 binds the CLEC14A with at least 5, or at least 10 or at least 50 times greater affinity than for thrombomodulin. More preferably, the portion of MMRN2 binds the CLEC14A with at least 100, or at least 1,000, or at least 10,000 times greater affinity than for thrombomodulin. Such binding may be determined by methods well known in the art, such as one of the Biacore® systems.

Typically, the portion of MMRN2 that selectively binds to CLEC14A binds to the mature polypeptide (residues 22-490) and not to the signal peptide (residues 1-21). As shown in Example 1, the inventors have identified an anti-angiogenic portion of MMRN2 that binds to the CLEC14A C-type lectin domain (residues 32-173), and more specifically to the long-loop region of CLEC14A (residues E97-C143 and even more specifically to the region spanning amino acids 97-108 of human CLEC14A, namely ERRRSHCTLENE. Hence, it is preferred if the portion of MMRN2 selectively binds to the C-type lectin domain (residues 32-173) of CLEC14A, and it is more preferred if the portion binds to the long loop region of CLEC14A (residues E97-C143), and it is even more preferred if the portion binds to a region of CLEC14A corresponding to the region spanning amino acid residues 97-108 of the human CLEC14A polypeptide.

By the region corresponding to the region spanning amino acid residues 97-108 of human CLEC14A, we include the meaning of a region in a CLEC14A orthologue or variant that aligns to the region spanning amino acid residues 97-108 when the sequence of the CLEC14A orthologue or variant is compared to the human CLEC14A sequence by alignment. Of course, when the portion is one that binds to human CLEC14A, the region corresponding to the region spanning amino acid residues 97-108 will be the region in the human CLEC14A itself.

Whether or not a given portion selectively binds to the C-type lectin domain or the region spanning amino acids 97-108 can be determined using routine methods in the art such as epitope mapping, competition binding studies, and chimera studies as described in Example 1.

By a portion of MMRN2 that selectively binds to a specific portion of CLEC14A, we include the meaning that not only does the portion of MMRN2 selectively bind to the target as described above, the portion of MMRN2 also binds the specified region of the CLEC14A with a greater affinity than for any other region of it. Preferably, the portion of MMRN2 binds the specified region with at least 2, or at least 5, or at least 10 or at least 50 times greater affinity than for any other region on CLEC14A. More preferably, the portion of MMRN2 binds the specified region with at least 100, or at least 1,000, or at least 10,000 times greater affinity than for than for any other region on the CLEC14A. Such binding may be determined by methods well known in the art, such as one of the Biacore® systems. It is preferred if the portion of MMRN2 has an affinity for CLEC14A of at least 10⁻³ M, 10⁻⁴ M, 10 ⁻⁵ M, 10⁻⁶ M or higher. Preferably, the portion of MMRN2 selectively binds the particular specified region within the CLEC14A and does not bind any other regions within it. Thus, in a particularly preferred embodiment, the portion of MMRN2 selectively binds to the region of CLEC14A corresponding to the region spanning amino acids residues 97-108 of human CLEC14A, and does not bind to, or makes only weak interactions with, amino acids outside of this region. For example, mutating amino acids residues other than residues 97-108 would not be expected to significantly affect binding of the portion of MMRN2 (e.g. reduce it to less than 90%, 80%, 70%, 60% or 50% of the original level of binding). Of course, it will be appreciated that mutations outside of the region spanning amino acids 97-108 may affect the overall structure and folding of the CLEC14A which may have an effect on binding of the portions of MMRN2. However, where the mutations outside of this region do not affect the global structure or folding of the protein, they are not expected to significantly affect binding of the portion of MMRN2 to CLEC14A.

The inventors have shown that the anti-angiogenic portion of MMRN2 described in Example 1 to CLEC14A is dependent on cysteine residues 103 and 138 of human CLEC14A, and so in a preferred embodiment, the portion of MMRN2 of the first aspect of the invention does not bind to a mutant CLEC14A polypeptide in which the cysteine corresponding to cysteine-103 of human CLEC14A is mutated and/or the cysteine corresponding to cysteine-138 of human CLEC14A is mutated. By cysteine corresponding to cysteine-103 or cysteine-138 of human CLEC14A, we include the meaning of cysteines in other CLEC14A orthologues or variants that align to cysteine-103 or cysteine-138 in human CLEC14A when the CLEC14A orthologue or variant and human CLEC14A are compared by alignment. Of course, when the CLEC14A is human CLEC14A, the cysteines corresponding to cysteine-103 and cysteine-138 will be cysteine-103 and cysteine-138.

In one embodiment, the portion of MMRN2 of the first aspect of the invention comprises or consists of the coiled-coil domain of MMRN2, or part thereof. The coiled-coil domain of human MMRN2 corresponds to amino acid residues 133-820, and the coiled-coil domain in other MMRN2 orthologues or variants can be readily identified by alignment. Thus, the portion of MMRN2 may comprise or consist of contiguous portion of at least 10, 20, 30, 40, 50, 60, 70, 80, 90, 100, 110, 120, 130, 140, 150, 160, 170, 180, 190 or 200 amino acids of the coiled-coil domain of MMRN2.

In a preferred embodiment, the portion of MMRN2 comprises or consists of a region of MMRN2 corresponding to the region spanning amino acid residues 487-820 or 487-674 or 495-678 or 495-674 or 530-624 or 588-620 of human MMRN2, or a part thereof. Generally, the part thereof, is at least 10, 20, 30, 35, 40, 50, 60, 70, 80, 90, 100, 110, 120, 130, 140 or 150, consecutive amino acids in length. By corresponding region, we include the meaning of a region which aligns to one of the specified regions of human MMRN2 when a MMRN2 orthologue or variant and human MMRN2 polypeptides are compared by alignment. Of course, when the portion of MMRN2 is a portion of human MMRN2, it is preferred if the portion comprises or consists of the region spanning amino acid residues 487-820 or 487-674 or 495-678 or 495-674 or 530-624 or 588-620 of human MMRN2, or a part thereof.

In a particularly preferred embodiment, the portion of MMRN2 comprises or consists of a region corresponding to the region spanning amino acid residues 495-674 of human MMRN2, or a part thereof. Hence, the portion of MMRN2 may comprise or consist of at least 10, 20, 30, 40, 50, 60, 70, 80, 90, 100, 110, 120, 130, 140, 150, 160, 170 or 175 consecutive amino acids of the region corresponding to the region spanning amino acid residues 495-674 of human MMRN2, or may comprise or consist of the entire region corresponding to the region spanning amino acid residues 495-674 of human MMRN2. It will be appreciated that small portions of MMRN2 may be particularly desirable provided that they are expressed and are folded correctly. As stated above, the portion may be a portion of a variant of MMRN2, and so it will be appreciated that these particular portions (e.g. those that comprise or consist of a region corresponding to the region spanning amino acid residues 495-674 of human MMRN2 or part thereof) may have at least 30%, 40%, 50%, 60%, or 70% sequence identity to the amino acid sequence of the corresponding portion of the wild type MMRN2 (e.g. human MMRN2), and preferably 75%, 80%, 85%, 90%, 95%, 96%, 97%, 98% or 99% sequence identity.

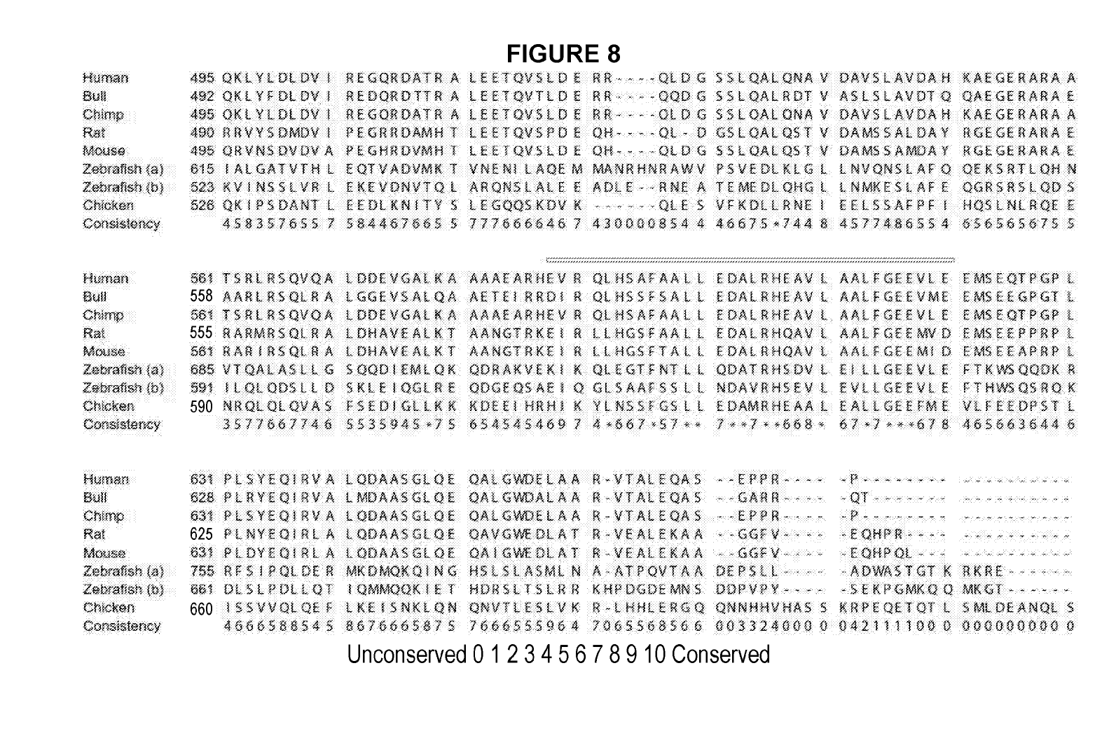

The inventors have aligned human MMRN2 with orthologues from different species within the region spanning amino acid residues 495-674, as illustrated in FIG. 8. The most conserved residues are Leu-536, Val-575, Leu-578, Val-589, Leu-592, Phe-596, Leu-599, Leu-600, Asp-602, Ala-603, Arg-605, His-606, Leu-610, Leu-613, Gly-615, Glu-616, Glu-617, and Leu-658, and the next most conserved residues are Leu-497, Glu-506, Gln-527, Val-540, Ala-546, Val-609, Glu-620, Gln-636, Ile-637, Leu-641, Leu-648 and Glu-666.

Thus, it is particularly preferred if the portion of MMRN2, or of a variant thereof, comprises one or more (for example at least 2, 3, 4, 5, 6, 7, 8, 9, 10, 11, 12, 13, 14, 15, 16 of 17 of or all 18) of amino acids corresponding to any of the following amino acids according to the numbering of human MMRN2 in FIG. 15: Leu-536, Val-575, Leu-578, Val-589, Leu-592, Phe-596, Leu-599, Leu-600, Asp-602, Ala-603, Arg-605, His-606, Leu-610, Leu-613, Gly-615, Glu-616, Glu-617, and Leu-658; and/or one or more (for example at least 2, 3, 4, 5, 6, 7, 8, 9, 10, or 11 of or all 12) of amino acids corresponding to any of the following amino acids according to the numbering of human MMRN2 in FIG. 15: Leu-497, Glu-506, Gln-527, Val-540, Ala-546, Val-609, Glu-620, Gln-636, Ile-637, Leu-641, Leu-648 and Glu-666.

It will be appreciated that when the portion (e.g. portion of the MMRN2 that comprises or consists of a region corresponding to the region spanning amino acid residues 495-674 of human MMRN2, or part thereof) is a portion of a variant of MMRN2, it is preferred if the particular residues listed above (i.e. Leu-536, Val-575, Leu-578, Val-589, Leu-592, Phe-596, Leu-599, Leu-600, Asp-602, Ala-603, Arg-605, His-606, Leu-610, Leu-613, Gly-615, Glu-616, Glu-617, and Leu-658 and/or Leu-497, Glu-506, Gln-527, Val-540, Ala-546, Val-609, Glu-620, Gln-636, Ile-637, Leu-641, Leu-648 and Glu-666) are not substituted for another amino acid. In other words, it is preferred if the portion of a variant of MMRN2 comprises all of the recited amino acids.

As seen from FIG. 8, there is a highly conserved region in MMRN2 orthologues that corresponds to the region spanning residues 588-620 of human MMRN2. Thus, it is preferred if any portion of MMRN2 comprises the region corresponding to the region spanning residues 588-620 of human MMRN2.

The inventors have found that the conserved region has the consensus sequence BₙX¹[V/L]X²X³LX⁴X⁵X⁶FX⁷X⁸LLX⁹DAX¹⁰RHX¹¹X¹²X¹³LX¹⁴X¹⁵LX¹⁶GEEX¹⁷X¹⁸X¹⁹Jᵣ (SEQ ID NO: 1) wherein B is a first chemical moiety, X¹-X¹⁹ are any amino acid, J is a second chemical moiety, n=0 or 1, and r=0 or 1 (Formula I) and so in a particularly preferred embodiment, the portion of MMRN2 of the first aspect of this invention has the structure BₙX¹[V/L]X²X³LX⁴X⁵X⁶FX⁷X⁸LLX⁹DAx¹⁰RHX¹¹X¹²X¹³LX¹⁴X¹⁵LX¹⁶GEEX¹⁷X¹⁸X¹⁹Jᵣ (SEQ ID NO: 1) wherein B is a first chemical moiety, X¹-X¹⁹ are any amino acid, J is a second chemical moiety, n=0 or 1, and r=0 or 1 (Formula I).

By [V/L], we mean that the peptide has a valine or a leucine residue at the specified position.

In a preferred embodiment (SEQ ID NO: 2):

X¹ is any basic amino acid, such as Glu, Asp, Lys or His;

X² is Arg or Lys or Gln;

X³ is any amino acid;

X⁴ is His or Glu or Ser or Asn;

X⁵ is Ser or Gly or Ala;

X⁶ is Ala or Ser or Thr;

X⁷ is any amino acid;

X⁸ is Ala or Thr or Ser;

X⁹ is Glu or Gln or Asn;

X¹⁹ is Leu or Thr or Val or Met;

X¹¹ is Glu or Gln or Ser;

X¹² is Ala or Asp or Glu;

X¹³ is Val or Ala;

X14 is Ala or Glu;

X¹⁵ is Ala or Ile or Val;

X¹⁶ is Phe or Leu;

X¹⁷ is Val or Met or Phe;

x18 is Leu or Met or Val or Ile; and

X¹⁹ is Glu or Asp.

optionally, wherein the moieties of each of B and J comprise one or more amino acids.

In a particularly preferred embodiment, X¹ is Glu, X² is Arg, X³ is any amino acid (preferably Gln), X⁴ is His, X⁵ is Ser, X⁶ is Ala, X⁷ is any amino acid (preferably Ala), X⁸ is Ala, X⁹ is Glu, X¹⁰ is Leu, X¹¹ is Glu, X¹² is Ala, X¹³ is Val, X¹⁴ is Ala, X¹⁵ is Ala, X¹⁶ is Phe, X¹⁷ is Val, X¹⁸ is Leu and X¹⁶ is Glu, and so the portion of MMRN2 may have the structure BₙE[V/L]RX³LHSAFX⁷ALLEDALRHEAVLAALFGEEVLEJᵣ (SEQ ID NO: 3).

The moieties of each of B and J may comprise one or more amino acids, and so may correspond to regions of the MMRN2 amino acid sequence, or variants thereof, that flank the conserved region defined by residues 588-620. However, it will be appreciated that either of moieties B and J may be any other chemical moiety, including for example chemical modifications, such as those intended to stabilise the portion of MMRN2. Suitable such modification are described in more detail below.

In an embodiment, the portion of MMRN2 is a peptide of no more than 180 amino acids (eg no more than 170, 160, 150, 140, 130, 120, 110, 100 or 50 amino acids) that comprises the structure

BₙX¹[V/L]X²X³_{LX}⁴X⁵X⁶FX⁷X⁸LLX⁹DAX¹⁰RHX¹¹X¹²X¹³LX¹⁴X₁₅LX¹⁶GEEX¹⁷X¹⁸X¹⁹Jᵣ (SEQ ID NO: 1) wherein B is a first chemical moiety, X¹-X¹⁹ are any amino acid, J is a second chemical moiety, n=0 or 1, and r=0 or 1. Preferences for X¹-X¹⁹ are listed above. Hence, the portion of MMRN2 may be a peptide of no more than 180 amino acids that comprises the structure BₙE[V/L]RX³LHSAFX⁷ALLEDALRHEAVLAALFGEEVLEJᵣ (SEQ ID NO: 3). X³ and X⁷ are both any amino acid, but are preferably Gln and Ala, respectively.

In a preferred embodiment, the portion of MMRN2 comprises or consists of the amino acid sequence of any of the MMRN2 portions listed in FIG. 8, or any part or variant of said portions. It is especially preferred if the portion of MMRN2 comprises or consists of the amino acid sequence of the human MMRN2 portion listed in FIG. 8, or a part of variant thereof.

By “part of said portion”, we include the meaning of a contiguous part of one of the MMRN2 portions listed in FIG. 8 of at least 10 amino acids in length, such as at least, 20, 30, 40, 50, 60, 70, 80, 90, 100, 110, 120, 130, 140, 150, 160, 170, or 175 amino acids in length. Typically, the part is no more than 180 amino acids in length. Preferably, the part of said portion (e.g. one no more than 180 amino acids in length) comprises the region of MMRN2 corresponding to the region spanning amino acid residues 588-620 of human MMRN2.

By a “variant of said portion”, we include the meaning of a variant having at least 30% sequence identity to one of the MMRN2 portions listed in FIG. 8, such as at least 40%, 50%, 60% or 70% sequence identity, and more preferably, 75%, 80%, 85%, 90%, 95% or 99% sequence identity.

As described in Example 1, the inventors have shown that portions of MMRN2 that inhibit the interaction between MMRN2 and CLEC14A have anti-angiogenic properties as well as anti-cancer properties. In particular, a portion of MMRN2 was shown to inhibit tube formation, and inhibited tumour growth in mice with Lewis lung carcinoma. Thus, in one embodiment of this aspect of the invention, the portion of MMRN2 is one that inhibits angiogenesis, for example as demonstrated in an angiogenesis assay, and/or is one that inhibits tumour growth, for example as demonstrated in an animal model of cancer (e.g. a mouse with Lewis lung carcinoma). Suitable angiogenesis assays are well known in the art and include an aortic ring assay, a sponge angiogenesis assay, an assay of endothelial cell proliferation, an assay of endothelial cell migration and/or an assay of endothelial cell invasion. Likewise, suitable animal models of cancer are well known in the art. It will be appreciated that assessment of anti-angiogenic properties and anti-cancer properties can be carried out in vitro or in vivo.

Conveniently, the portion of MMRN2 molecule may be modified so that it can be more easily detected, for example by biotinylating it or by incorporating any detectable label known in the art such as affinity tags, radiolabels, fluorescent labels or enzymatic labels. The incorporation of affinity tags into the portion of MMRN2 is discussed in more detail below.

The portion of MMRN2 is typically made by recombinant DNA technology. Suitable techniques for cloning, manipulation, modification and expression of nucleic acids, and purification of expressed proteins, are well known in the art and are described for example in Sambrook et a! (2001) “Molecular Cloning, a Laboratory Manual”, 3rd edition, Sambrook et al (eds), Cold Spring Harbor Laboratory Press, Cold Spring Harbor, N.Y., USA. Alternatively, the portion of MMRN2 may be made using protein chemistry techniques for example using partial proteolysis (either exolytically or endolytically), or by de novo synthesis.

The amino acid residues described herein may be in the “L” isomeric form. However, residues in the “D” isomeric form can be substituted for any L-amino acid residue, as long as the portion of MMRN2 can still inhibit the interaction between MMRN2 and CLEC14A. This definition includes, unless otherwise specifically indicated, chemically-modified amino acids, including amino acid analogues (such as penicillamine, 3-mercapto-D-valine), naturally-occurring non-proteogenic amino acids (such as norleucine), and chemically-synthesised compounds that have properties known in the art to be characteristic of an amino acid. The term “proteogenic” indicates that the amino acid can be incorporated into a protein in a cell through well-known metabolic pathways.

The portion of MMRN2 can be a peptide “mimetic”, i.e. peptidomimetics which mimic the structural features of the portion comprising or consisting of the amino acid sequence as described above. Retro-inverso peptides (known as all-D-retro or retro-enantio peptides) are also included, whereby all of the L-amino acids are replaced with D-amino acids and the peptide bonds are reversed.

Peptidomimetics that are non-peptide in nature can be designed and synthesised by standard organic chemical methods. Peptidomimetics that are non-peptide in nature can be even more advantageous in therapeutic use, in the resistance to degradation, in permeability and in possible oral administration.

Peptidomimetics are small molecules that can bind to proteins by mimicking certain structural aspects of peptides and proteins. They are used extensively in science and medicine as agonists and antagonists of protein and peptide ligands of cellular and other receptors, and as substrates and substrate analogues for enzymes. Some examples are morphine alkaloids (naturally-occurring endorphin analogues), penicillins (semi-synthetic), and HIV protease inhibitors (synthetic). Such compounds have structural features that mimic a peptide or a protein and as such are recognised and bound by other proteins. Binding the peptidomimetic either induces the binding protein to carry out the normal function caused by such binding (agonist) or disrupts such function (antagonist, inhibitor).

It is appreciated that the portion of MMRN2 may be modified. For example, a derivative of the portion of MMRN2 described herein may be useful to inhibit angiogenesis and/or combat cancer in an individual as described in more detail below. By “derivative”, we include the meaning of the portion of MMRN2 having one or more residues chemically derivatised by reaction of a functional side group. Such derivatised molecules include, for example, those molecules in which free amino groups have been derivatised to form amine hydrochlorides, p-toluene sulfonyl groups, carbobenzoxy groups, t-butyloxycarbonyl groups, chloroacetyl groups or formyl groups. Free carboxyl groups may be derivatised to form salts, methyl and ethyl esters or other types of esters or hydrazides. Free hydroxyl groups may be derivatised to form O-acyl or 0-alkyl derivatives. Also included as derivatives are those peptide portions that contain one or more naturally occurring amino acid derivatives of the twenty standard amino acids. For example, 4-hydroxyproline may be substituted for proline; 5-hydroxylysine may be substituted for lysine; 3-methylhistidine may be substituted for histidine; homoserine may be substituted for serine; and ornithine may be substituted for lysine. The derivatisation does not include changes in functional groups which change one amino acid to another.

Some useful modifications are designed to increase the stability and, therefore, the half-life of polypeptides or peptides in solutions, particularly biological fluids, such as blood, plasma or serum, by blocking proteolytic activity in the blood. Thus, the portion of MMRN2 may have a stabilising group at one or both termini. Typical stabilising groups include amido, acetyl, benzyl, phenyl, tosyl, alkoxycarbonyl, alkyl carbonyl, benzyloxycarbonyl and the like end group modifications. Additional modifications include using a “D” amino acid in place of a “L” amino acid at the termini, and amide rather than amino or carboxy termini to inhibit exopeptidase activity. Thus, it is appreciated that the portion of MMRN2 may have a capping moiety at one or both ends, preferably a moiety that is less than 100 Da in molecular weight such as an amide group. It will be appreciated that such a modification may correspond to at least part of moieties B and/or J in the formulae above.

Agents that Inhibit the Interaction Between CD93 and MMRN2

The inventors have shown that MMRN2 interacts with CD93, that the interaction plays a role in angiogenesis and that portions of MMRN2 that inhibit the interaction between MMRN2 and CD93 have anti-angiogenic properties as well as anti-cancer properties. Accordingly, a second aspect of the invention provides an agent that inhibits the interaction between CD93 and MMRN2. Preferably, the agent is a portion of MMRN2 or a variant thereof, that inhibits the interaction between CD93 and MMRN2.

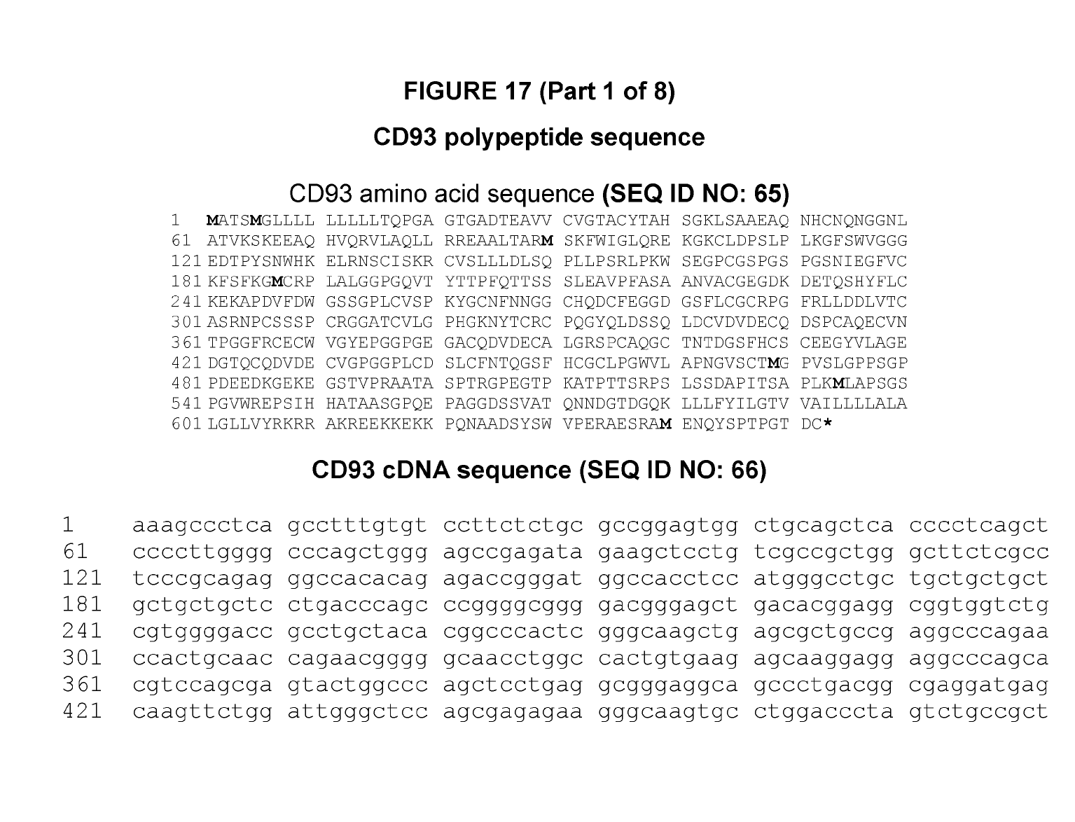

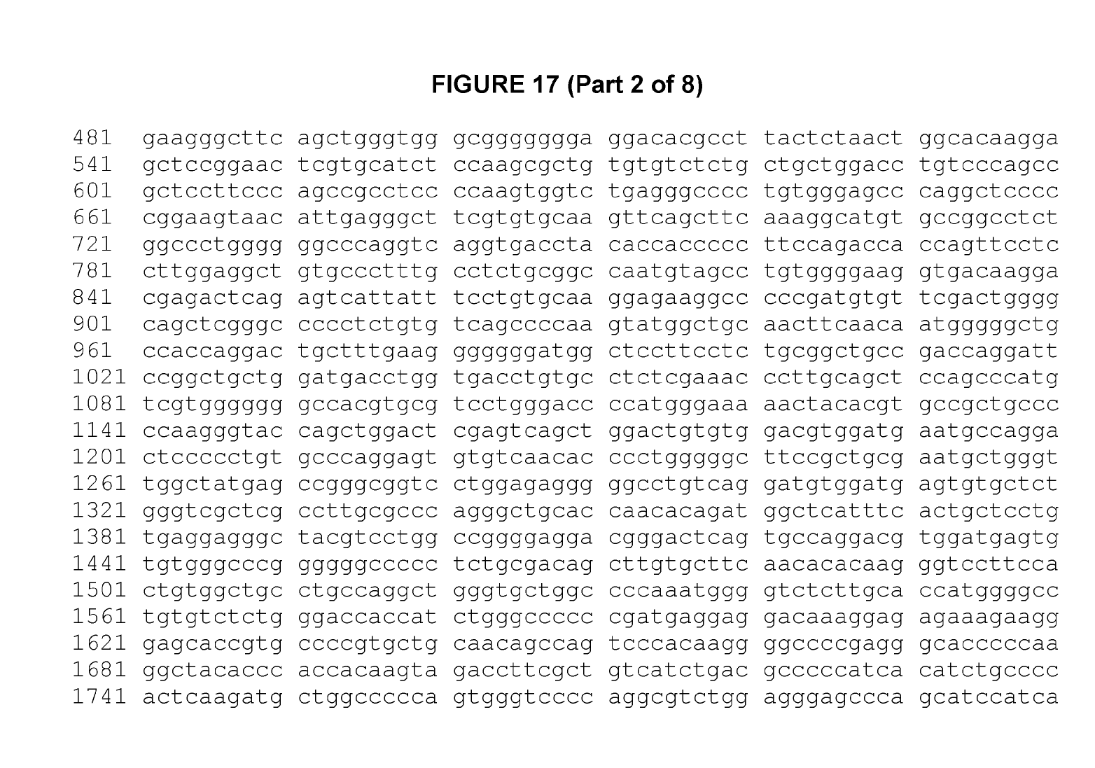

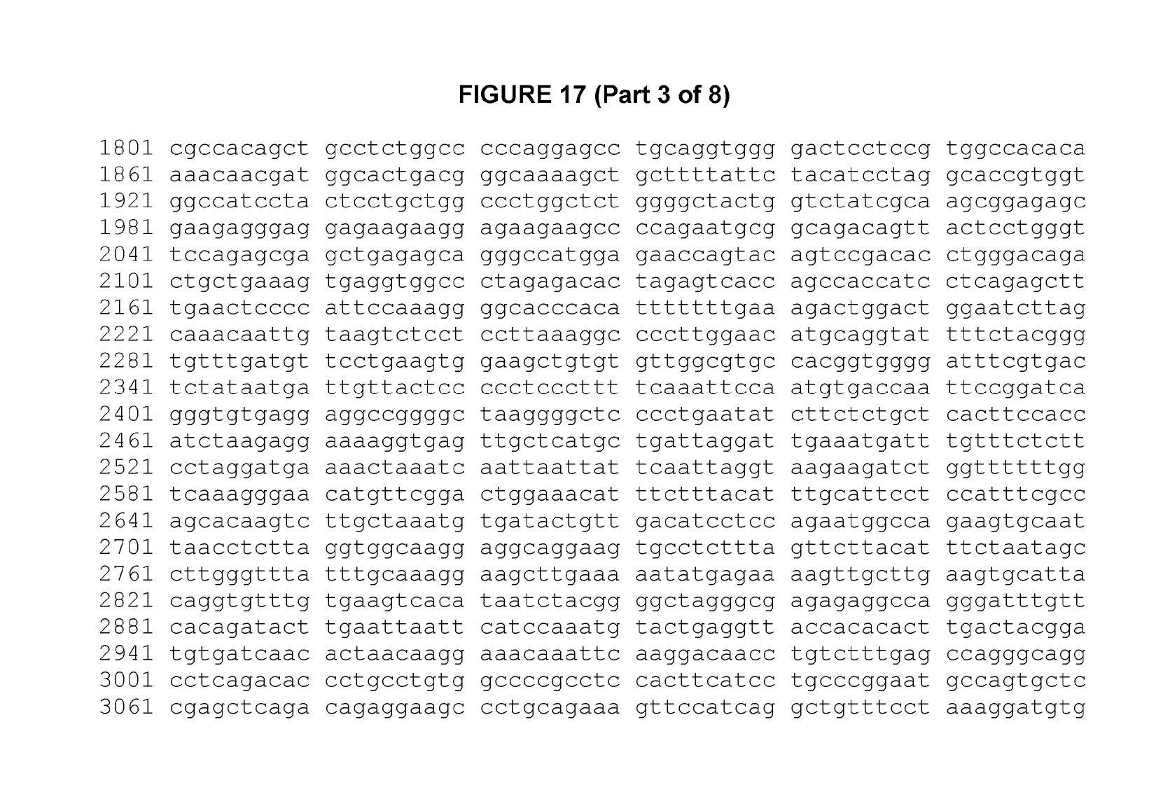

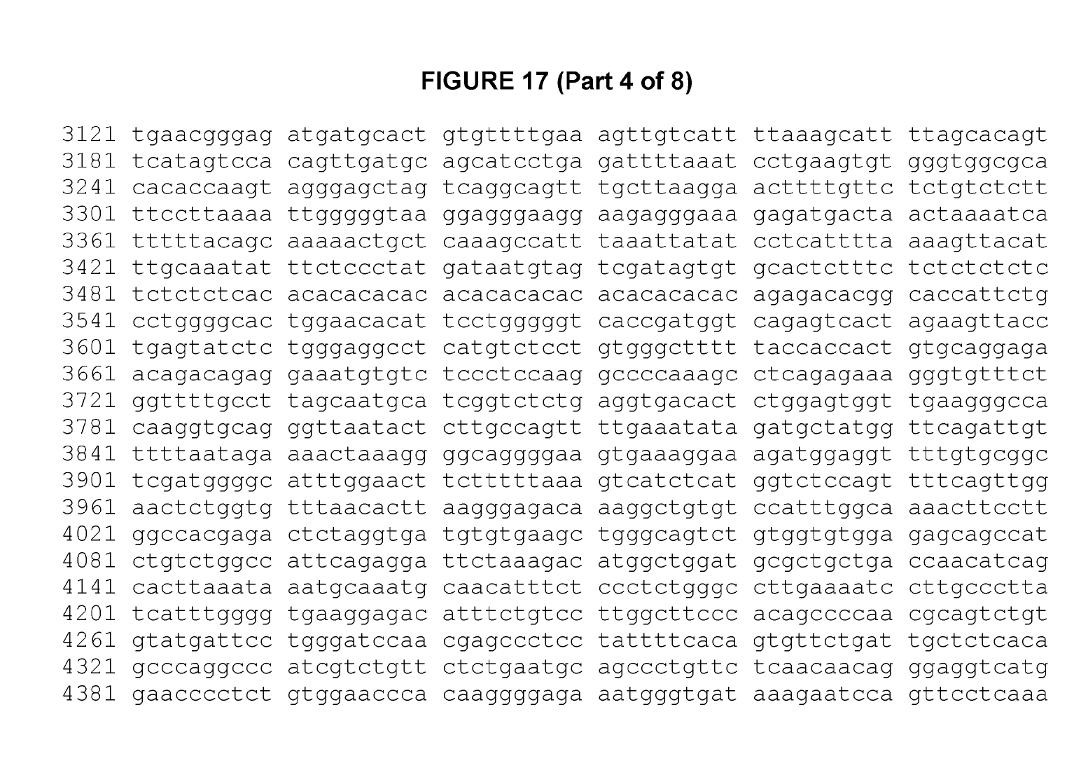

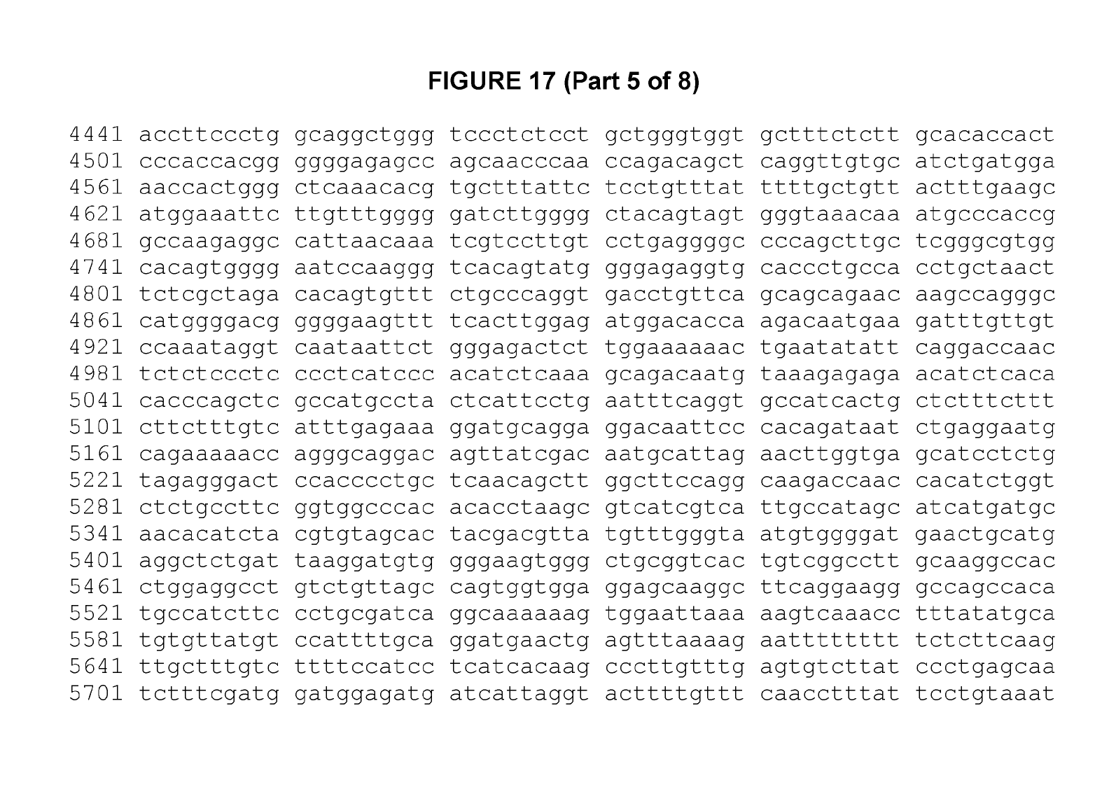

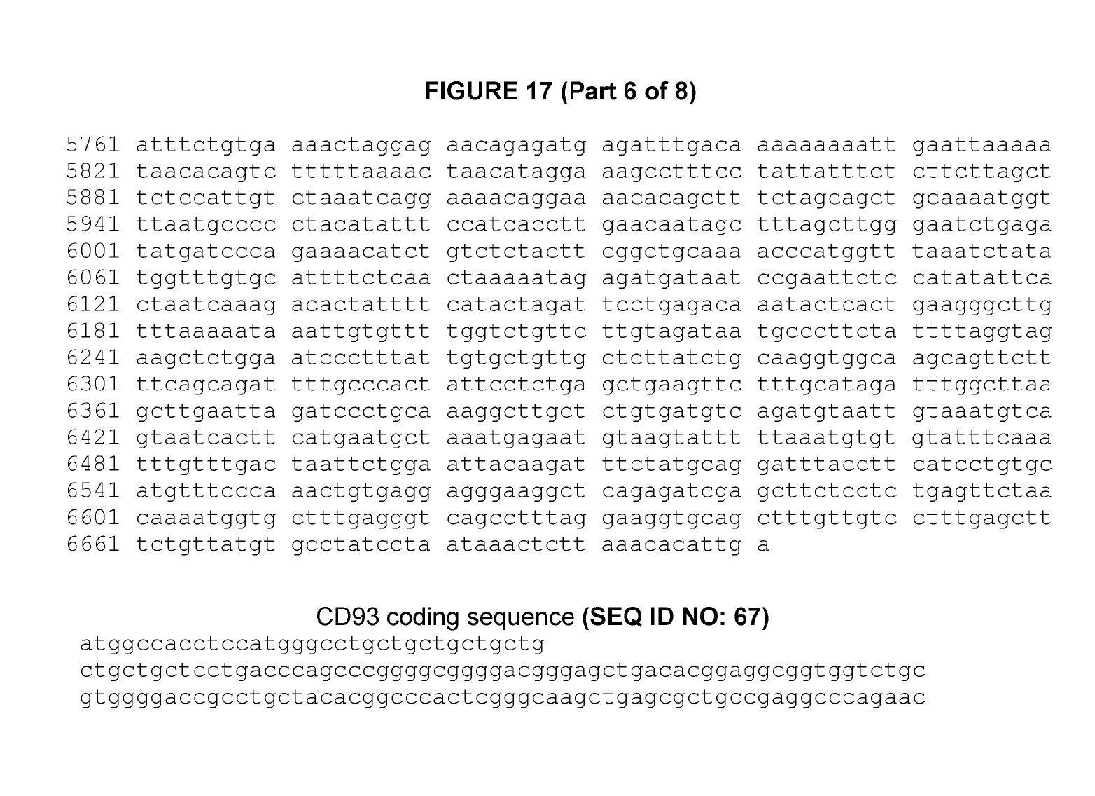

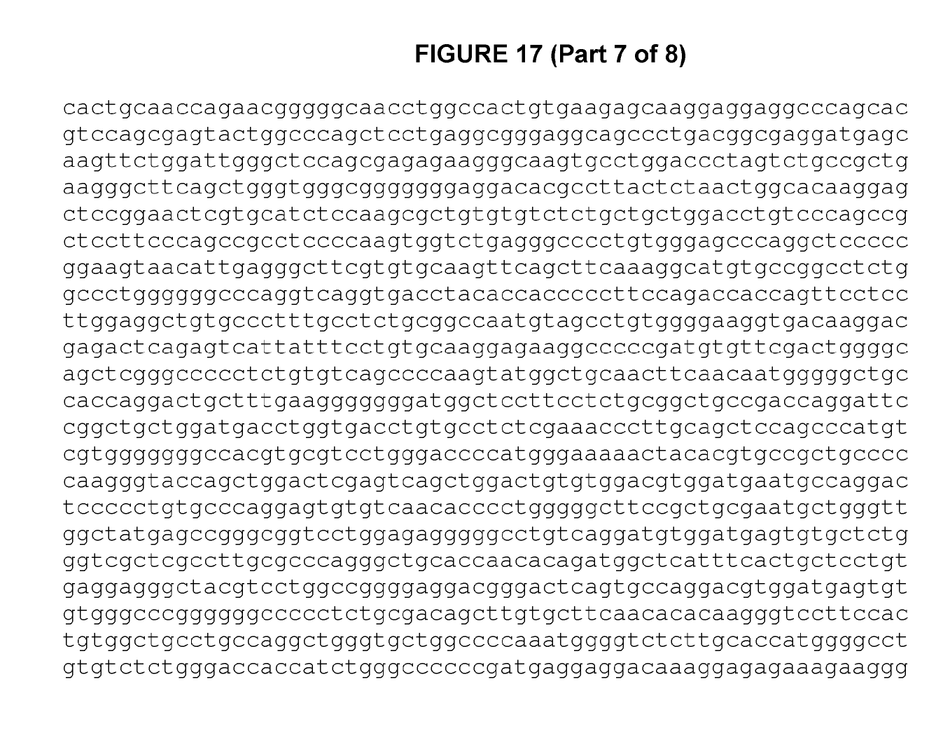

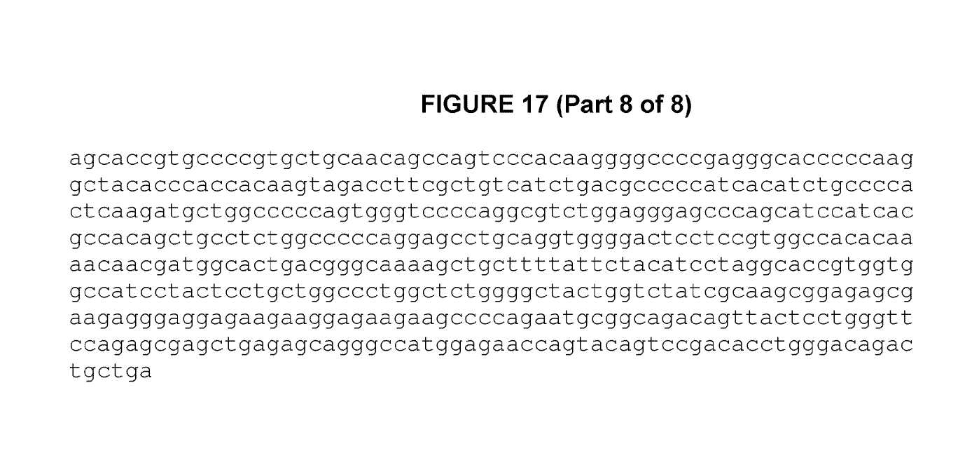

The gene CD93 is located at 20p11.21 and encodes a 652 amino acid residue polypeptide. By the CD93 polypeptide we include the meaning of a gene product of human CD93, including naturally occurring variants thereof. Human CD93 polypeptide includes the amino acid sequence found in Genbank Accession No NP_036204.2 and naturally occurring variants thereof. The CD93 polypeptide sequence from NP_036204.2 is shown in FIG. 17. Also included are CD93 orthologues found in other species, such as in horse, bull, chimp, chicken, zebrafish, dog, pig, cow, sheep, rat, mouse, guinea pig or a primate.

By “natural variants” we include, for example, allelic variants. Typically, these will vary from the given sequence by only one or two or three, and typically no more than 10 or 20 amino acid residues. Typically, the variants have conservative substitutions.

A cDNA sequence corresponding to a human CD93 mRNA is found in Genbank Accession No NM_012072.3, is also shown in FIG. 17.

By an agent that inhibits the interaction between CD93 and MMRN2, we include the meaning of an agent that reduces the level of binding between CD93 and MMRN2, as compared to the level of binding between CD93 and MMRN2 in the absence of the agent. Preferably, the agent is one that reduces the level of binding between CD93 and MMRN2 by at least 10%, 20%, 30%, 40% or 50%, and more preferably the agent is one that reduces the level of binding between CD93 and MMRN2 by at least 70%, 80%, 90%, 95% or 99%. Most preferably, the agent is one that reduces the level of binding between CD93 and MMRN2 to an undetectable level, or eliminates binding between CD93 and MMRN2.

Suitable methods for detecting and/or measuring (quantifying) the binding of CD93 to MMRN2 are well known to those skilled in the art, and include those described above.

The agent may be any of an antibody, a polypeptide, a peptide, a polynucleotide, a peptidomimetic, a natural product, a carbohydrate, an aptamer or a small molecule. Particular examples of what the agent may be are described below, and methods for identifying suitable agents feature in a subsequent aspect of the invention.

It is appreciated that the agent itself may inhibit the interaction between CD93 and MMRN2 directly (eg by binding to CD93 or MMRN2).

It will be appreciated that polypeptide agents that inhibit the interaction between CD93 and MMRN2 may be administered directly, or may be administered in the form of a polynucleotide that encodes the agent. Thus, as used herein, unless the context demands otherwise, by administering to the individual an agent that inhibits the interaction between CD93 and MMRN2 which agent is a polypeptide, we include the meanings of administering the inhibitor directly, or administering a polynucleotide that encodes the inhibitor, typically in the form of a vector. Similarly, as used herein, unless the context demands otherwise, by a medicament or a composition comprising an agent that inhibits the interaction between CD93 and MMRN2 which is a polypeptide, we include the meanings that the medicament or composition comprises the agent itself, or comprises a polynucleotide that encodes the agent.

For the avoidance of doubt, by an agent that inhibits the interaction between CD93 and MMRN2, we also include the meaning of prodrugs thereof. For example, the agent may be administered as a prodrug which is metabolised or otherwise converted into its active form once inside the body of a subject. The term “prodrug” as used in this application refers to a precursor or derivative form of a pharmaceutically active substance that is less active compared to the parent drug and is capable of being enzymatically activated or converted into the more active parent form (see, for example, D. E. V. Wilman “Prodrugs in Cancer Chemotherapy” Biochemical Society Transactions 14, 375-382 (615th Meeting, Belfast 1986) and V. J. Stella et al. “Prodrugs: A Chemical Approach to Targeted Drug Delivery” Directed Drug Delivery R. Borchardt et al (ed.) pages 247-267 (Humana Press 1985)).

Agents that are especially active at inhibiting tumour angiogenesis are preferred for anti-cancer therapeutic agents, and they can be selected for this activity using methods well known in the art and as described below.

It will be appreciated that the agent may be an antibody that inhibits the interaction between CD93 and MMRN2.

The antibody may be one that binds specifically to regions of CD93 and/or MMRN2 that are involved either directly or indirectly in the interaction between CD93 and MMRN2. For example, the antibody may bind to the MMRN2 binding site in CD93 and so directly block binding of MMRN2, or the antibody may bind to a region of CD93 outside the MMRN2 binding site that is nevertheless required for a stable interaction and so indirectly affects binding to MMRN2. Similarly, the antibody may bind to the CD93 binding site in MMRN2 and so directly block binding of CD93, or the antibody may bind to a region of MMRN2 outside the CD93 binding site that is nevertheless required for a stable interaction and so indirectly affects binding to CD93.

Suitable antibodies which bind to CD93 or MMRN2, or to specified portions thereof, can be made by the skilled person using technology long-established in the art. Methods of preparation of monoclonal antibodies and antibody fragments are well known in the art and include hybridoma technology (Kohler & Milstein (1975) “Continuous cultures of fused cells secreting antibody of predefined specificity. Nature 256: 495-497); antibody phage display (Winter et al (1994) “Making antibodies by phage display technology.” Annu. Rev. Immunol. 12: 433-455); ribosome display (Schaffitzel et al (1999) “Ribosome display: an in vitro method for selection and evolution of antibodies from libraries.” J. Immunol. Methods 231: 119-135); and iterative colony filter screening (Giovannoni et al (2001) “Isolation of anti-angiogenesis antibodies from a large combinatorial repertoire by colony filter screening.” Nucleic Acids Res. 29: E27). Further, antibodies and antibody fragments suitable for use in the present invention are described, for example, in the following publications: “Monoclonal Hybridoma Antibodies: Techniques and Application”, Hurrell (CRC Press, 1982); “Monoclonal Antibodies: A Manual of Techniques”, H. Zola, CRC Press, 1987, ISBN: 0-84936-476-0; “Antibodies: A Laboratory Manual” 1st Edition, Harlow & Lane, Eds, Cold Spring Harbor Laboratory Press, New York, 1988. ISBN 0-87969-314-2; “Using Antibodies: A Laboratory Manual” 2nd Edition, Harlow & Lane, Eds, Cold Spring Harbor Laboratory Press, New York, 1999. ISBN 0-87969-543-9; and “Handbook of Therapeutic Antibodies” Stefan Dübel, Ed., 1st Edition,—Wiley-VCH, Weinheim, 2007. ISBN: 3-527-31453-9.

By an antibody that selectively binds to CD93 or MMRN2, we include the meaning that the antibody molecule binds CD93 or MMRN2 with a greater affinity than for an irrelevant polypeptide, such as human serum albumin (HSA). Preferably, the antibody binds the CD93 or MMRN2 with at least 5, or at least 10 or at least 50 times greater affinity than for the irrelevant polypeptide. More preferably, the antibody molecule binds the CD93 or MMRN2 with at least 100, or at least 1,000, or at least 10,000 times greater affinity than for the irrelevant polypeptide. Such binding may be determined by methods well known in the art, such as one of the Biacore® systems.

It is preferred that the antibody that selectively binds CD93 or MMRN2 does not bind a related polypeptide, such as thrombomodulin in the case of CD93 or multimerin 1 in the case of MMRN2, or that the antibody molecule binds CD93 or MMRN2 with a greater affinity than for the related polypeptide, such as thrombomodulin in the case of CD93 or multimerin 1 in the case of MMRN2. Preferably, the antibody binds the CD93 or MMRN2 with at least 5, or at least 10 or at least 50 times greater affinity than for the related polypeptide. More preferably, the antibody molecule binds the CD93 or MMRN2 with at least 100, or at least 1,000, or at least 10,000 times greater affinity than for the related polypeptide. Such binding may be determined by methods well known in the art, such as one of the Biacore® systems.

It is preferred if the antibodies have an affinity for CD93 or MMRN2 of at least 10⁻⁵ M, 10⁻⁶ M, or 10⁻⁷ M and more preferably 10⁻⁸ M, although antibodies with higher affinities, e.g. 10⁻⁹ M, or higher, may be even more preferred.

In a particularly preferred embodiment, the antibody is one that selectively binds to the CD93 polypeptide.

Typically, the antibody that selectively binds to CD93 binds to the mature peptide (residues 22-652) and not to the signal peptide (residues 1-21). Preferably, the antibody that selectively binds CD93 binds to the extracellular region of CD93 (residues A22-K580). The antibody may bind to the EGF-like region, but it is preferred if the antibody binds to the C-type lectin domain (A22-N174) or the long-loop region of CD93 (residues Q98-C142). More preferably, the antibody binds to the region spanning amino acid residues 97-108 of CD93 which is within the C-type lectin domain, namely QREKGKCLDPSL.

It is especially preferred if the antibody that selectively binds to the CD93 polypeptide, selectively binds to the MMRN2 binding region of the CD93 polypeptide within the C-type lectin domain. Thus, the antibody may be one that competes with MMNR2 for specific binding to the CD93 polypeptide. Whether or not a given antibody selectively binds to the MMRN2 binding region or competes with MMRN2 for specific binding to the CD93 polypeptide can be determined using routine methods in the art such as epitope mapping, and competition binding studies. For example, binding of CD93 to the given antibody can be assessed following pre-incubation with varying concentrations of MMRN2.

In another embodiment, the antibody is one that selectively binds to the MMRN2 polypeptide. Thus, the antibody may be one that competes with CD93 for specific binding to the MMRN2 polypeptide. In this embodiment, it is preferred if the antibody selectively binds to the CD93 binding region of the MMRN2 polypeptide. Again, whether or not a given antibody binds to the CD93 binding region of the MMRN2 polypeptide or competes with CD93 for specific binding to the MMRN2 polypeptide can be determined using routine methods in the art such as epitope mapping and competition binding studies.

By an antibody that selectively binds a specific portion of CD93 or MMRN2 we include the meaning that not only does the antibody selectively bind to the target as described above, the antibody molecule also binds the specified portion of the CD93 or MMRN2 with a greater affinity than for any other portion of it. Preferably, the antibody binds the specified portion with at least 2, or at least 5, or at least 10 or at least 50 times greater affinity than for any other epitope on CD93 or MMRN2. More preferably, the antibody molecule binds the specified portion with at least 100, or at least 1,000, or at least 10,000 times greater affinity than for than for any other epitope on the CD93 or MMRN2. Such binding may be determined by methods well known in the art, such as one of the Biacore® systems. It is preferred if the antibodies have an affinity for their target epitope on the CD93 or MMRN2 of at least 10⁻⁷ M and more preferably 10⁻⁸ M, although antibodies with higher affinities, e.g. 10⁻⁹ M, or higher, may be even more preferred. Preferably, the antibody selectively binds the particular specified epitope within the CD93 or MMRN2 and does not bind any other epitopes within it.

Preferably, when the antibody is administered to an individual, the antibody binds to the target CD93 or MMRN2 or to the specified portion thereof with a greater affinity than for any other molecule in the individual. Preferably, the antibody binds to (a specified portion of) the CD93 or MMRN2 with at least 2, or at least 5, or at least 10 or at least 50 times greater affinity than for any other molecule in the individual. More preferably, the agent binds the CD93 or MMRN2 (at the specific domain) with at least 100, or at least 1,000, or at least 10,000 times greater affinity than any other molecule in the individual. Preferably, the antibody molecule selectively binds the CD93 or MMRN2 without significantly binding other polypeptides in the body.

In a preferred embodiment of the second aspect of the invention the agent is a portion of MMRN2 or a variant thereof that inhibits the interaction between CD93 and MMRN2.

Preferably, the portion of MMRN2 that inhibits the interaction between CD93 and MMRN2 is also one that inhibits the interaction between CLEC14A and MMRN2, and so it will be appreciated that all of the preferences and limitations described above in relation to the first aspect of the invention (including all structural and functional properties of the portion of MMRN2) may equally apply to the portion of MMRN2 according to the second aspect of the invention.

By a “portion of MMRN2 or a variant thereof” in the context of the second aspect of the invention, we include the meaning of any portion of the full length native MMRN2 polypeptide or any portion of a variant of the full length native MMRN2 polypeptide, provided that that portion inhibits the interaction between MMRN2 and CD93. In other words, the portion of MMRN2 is one that reduces the level of binding between CD93 and MMRN2, as compared to the level of binding between CD93 and MMRN2 in the absence of the portion of MMRN2. Suitable methods of assessing the binding between CD93 and MMRN2 are known in the art and include those described above in relation to CLEC14A and MMRN2, such as pull-down assays, enzyme linked immunosorbent assays (ELISA), surface plasmon resonance assays, chip-based assays, immunocytofluorescence, yeast two-hybrid technology and phage display.

Preferably, the portion is one that reduces the level of binding between CD93 and MMRN2 by at least 10%, 20%, 30%, 40% or 50%, and more preferably the portion is one that reduces the level of binding between CD93 and MMRN2 by at least 70%, 80%, 90%, 95% or 99%. Most preferably, the portion is one that reduces the level of binding between CD93 and MMRN2 to an undetectable level, or eliminates binding between CD93 and MMRN2.

In a preferred embodiment, the portion of MMRN2 of the second aspect of the invention is one that binds to CD93, and more preferably is one that selectively binds to CD93. By a portion of MMRN2 that selectively binds to CD93, we include the meaning that the portion of MMRN2 binds CD93 with a greater affinity than for an irrelevant polypeptide such as human serum albumin. Preferably, the portion of MMRN2 binds CD93 with at least 5, or at least 10 or at least 50 times greater affinity than for the irrelevant polypeptide. More preferably, the portion of MMRN2 binds the CD93 with at least 100, or at least 1,000, or at least 10,000 times greater affinity than for the irrelevant polypeptide. Such binding may be determined by methods well known in the art, such as one of the Biacore® systems.

In a particularly preferred embodiment, the portion of MMRN2 of the second aspect of the invention is one that binds to CLEC14A, and more preferably is one that selectively binds to CLEC14A, as described above in relation to the first aspect of the invention.

It is preferred that the portion of MMRN2 does not bind to thrombomodulin, or that the portion of MMRN2 binds CD93 with a greater affinity than for thrombomodulin. Preferably, the portion of MMRN2 binds the CD93 with at least 5, or at least 10 or at least 50 times greater affinity than for thrombomodulin. More preferably, the portion of MMRN2 binds the CD93 with at least 100, or at least 1,000, or at least 10,000 times greater affinity than for thrombomodulin. Such binding may be determined by methods well known in the art, such as one of the Biacore® systems.

Typically, the portion of MMRN2 that selectively binds to CD93 binds to the mature polypeptide (residues 22-652) and not to the signal peptide (residues 1-21). Preferably, the portion of MMRN2 binds to the extracellular region of CD93 (residues A22-K580). The portion of MMRN2 may bind to the EGF-like region, but it is preferred if the portion of MMRN2 selectively binds to the C-type lectin domain of CD93 (residues A22-N174), and it is more preferred if the portion binds to the long-loop region of CD93 (residues Q98-C142), and it is still more preferred if the portion binds to a region of CD93 corresponding to the region spanning amino acid residues 97-108 of the human CD93 polypeptide.

By the region corresponding to the region spanning amino acid residues 97-108 of human CD93, we include the meaning of a region in a CD93 orthologue or variant that aligns to the region spanning amino acid residues 97-108 when the sequence of the CD93 orthologue or variant is compared to the human CD93 sequence by alignment. Of course, when the portion is one that binds to human CD93, the region corresponding to the region spanning amino acid residues 97-108 will be the region in the human CD93 itself.

By a portion of MMRN2 that selectively binds to a specific portion of CD93, we include the meaning that not only does the portion of MMRN2 selectively bind to the target as described above, the portion of MMRN2 also binds the specified region of the CD93 with a greater affinity than for any other region of it. Preferably, the portion of MMRN2 binds the specified region with at least 2, or at least 5, or at least 10 or at least 50 times greater affinity than for any other region on CD93. More preferably, the portion of MMRN2 binds the specified region with at least 100, or at least 1,000, or at least 10,000 times greater affinity than for than for any other region on the CD93. Such binding may be determined by methods well known in the art, such as one of the Biacore® systems. It is preferred if the portion of MMRN2 has an affinity for CD93 of at least 10⁻³ M, 10⁻⁴ M, 10⁻⁵ M, 10⁻⁶ M or higher. Preferably, the portion of MMRN2 selectively binds the particular specified region within the CD93 and does not bind any other regions within it.

The inventors have shown that binding of the anti-angiogenic portion of MMRN2 described in Example 1 is dependent on cysteine residues 104 and 136 of human CD93, and so in a preferred embodiment, the portion of MMRN2 of the second aspect of the invention does not bind to a mutant CD93 polypeptide in which the cysteine corresponding to cysteine-104 of human CD93 is mutated and/or the cysteine corresponding to cysteine-136 of human CD93 is mutated. By cysteine corresponding to cysteine-104 or cysteine-136 of human CD93, we include the meaning of cysteines in other CD93 orthologues or variants that align to cysteine-104 or cysteine-136 in human CD93 when the CD93 orthologue or variant and human CD93 are compared by alignment. Of course, when the CD93 is human CD93, the cysteines corresponding to cysteine-104 and cysteine-136 will be cysteine-104 and cysteine-136.

Other preferences for the structural and functional properties of the portion of MMRN2 of the second aspect of the invention include those described above in relation to the portion of MMRN2 of the first aspect of the invention. In particular, the portion of MMRN2 of the second aspect of the invention may be a peptide of no more than 180 amino acids in length that comprises the structure

BₙX¹[V/L]X²X³LX⁴X⁵X⁶FX⁷X⁸LLX⁹DAX¹⁰RHX¹¹X¹²X¹³LX¹⁴X¹⁵LX¹⁶GEEX¹⁷X¹⁸X¹⁹Jᵣ (SEQ ID NO: 1) wherein B is a first chemical moiety, X¹-X¹⁹ are any amino acid, J is a second chemical moiety, n=0 or 1, and r=0 or 1 (Formula I).

Fusion Proteins

A third aspect of the invention provides a fusion protein comprising a portion of MMRN2 according to the first aspect of the invention or a polypeptide agent that inhibits the interaction between CD93 and MMRN2 according to the second aspect of the invention (which itself may be a portion of MMRN2), wherein the fusion protein does not comprise wild-type MMRN2. Thus, the fusion protein does not comprise the full-length wild type MMRN2 or any naturally occurring variant thereof.

It will be appreciated that the fusion protein may comprise the portion of MMRN2 or a polypeptide agent that inhibits the interaction between CD93 and MMRN2, and an affinity tag. Affinity tags are well known in the art and are described in more detail below. Particularly preferred examples are a poly histidine tag and an Fc tag. However, it will be appreciated that the fusion protein may comprise the portion of MMRN2 or polypeptide agent, and any other polypeptide such as an antibody.

A fourth aspect of the invention provides a nucleic acid molecule encoding the portion of MMRN2 of the first aspect of the invention or a polypeptide agent of the second aspect of the invention (which itself may be a portion of MMRN2), or the fusion protein according to the third aspect of the invention. The nucleic acid molecule may be DNA or it may be RNA. Typically, it is comprised in a vector, such as a vector which can be used to express the said portion of MMRN2 or polypeptide agent or fusion protein.

Antibodies

A fifth aspect of the invention provides an antibody that selectively binds to a portion of MMRN2 according to the first or second aspect of the invention. Preferences for the portion of MMRN2 include those defined above in relation to the first and second aspects of the invention.

By “selectively binds to a portion of MMRN2”, we include the meaning that the antibody binds to the portion of MMRN2 with a greater affinity than for an irrelevant polypeptide such as human serum albumin. Preferably, the antibody binds to the portion of MMRN2 with at least 5, or at least 10 or at least 50 times greater affinity than for the irrelevant polypeptide. More preferably, the antibody binds to the portion of MMRN2 with at least 100, or at least 1,000, or at least 10,000 times greater affinity than for the irrelevant polypeptide.

It is preferred if the antibody that selectively binds to the portion of MMRN2 binds to that portion of MMRN2 with a greater affinity than for any other region of the MMRN2 polypeptide (ie amino acid residues of MMRN2 outside of the portion of MMRN2). Preferably, the antibody binds to the portion of MMRN2 with at least 2, or at least 5, or at least 10 or at least 50 times greater affinity than for any other region on MMRN2. More preferably, the antibody binds to the portion of MMRN2 with at least 100, or at least 1,000, or at least 10,000 times greater affinity than for than for any other region on the MMRN2. It is appreciated that the antibody may bind to amino acid residues outside of the portion of MMRN2. However, the interaction between the antibody and such amino acids is expected to be weaker than that between the antibody and the portion of MMRN2.

In an embodiment, the antibody selectively binds to the coiled-coil domain of MMRN2, or part thereof, optionally wherein the coiled-coil domain of MMRN2 corresponds to amino acid residues 133-820 of human MMRN2. For example, the antibody may selectively bind to a region of MMRN2 corresponding to the region spanning amino acid residues 487-820 or 487-674 or 495-678 or 495-674 or 530-624 or 588-620 of human MMRN2, or a part thereof.

It is particularly preferred if the antibody selectively binds to a region corresponding to the region spanning amino acid residues 495-674 of human MMRN2, or a part thereof. Thus, the antibody may selectively bind to a portion of MMRN2 (e.g. a peptide of no more than 180 amino acids in length) that has the structure

BₙX¹[V/L]X¹X²X³LX⁴NX⁵X⁶FX⁷X⁸LLX⁹DAX¹⁰RHX¹¹X¹²X¹³LX¹⁴X¹⁵LX¹⁶GEEX¹⁷X¹⁸X¹⁹Jᵣ (SEQ ID NO: 1) wherein B is a first chemical moiety, X¹-X¹⁹ are any amino acid, J is a second chemical moiety, n=0 or 1, and r=0 or 1 (Formula I).

In an embodiment, the antibody is one that does not interact with, or makes only weak interactions with, amino acids outside the region defined by amino acid residues 495-674 of human MMRN2. For example, mutating amino acid residues other than residues 495-674 would not be expected to significantly affect antibody binding (e.g. reduce it to less than 90%, 80%, 70%, 60% or 50% of the original level of binding).

In an embodiment, the antibody is one that does not compete with an antibody known to bind elsewhere within MMRN2 for specific binding to an epitope located within amino acid residues 495-674 of MMRN2.

As with the portions of MMRN2 of the invention, it is preferred if the antibodies of the invention inhibit the interaction between CLEC14A and MMRN2 and/or inhibit the interaction between CD93 and MMRN2. Thus, the antibody may be one that competes with CLEC14A and/or CD93 for specific binding to the MMRN2 polypeptide.

It is preferred if the antibodies have an affinity for the portion of MMRN2 of at least 10⁻⁵ M, 10⁻⁶ M, or 10⁻⁷ M and more preferably 10⁻⁸ M, although antibodies with higher affinities, e.g. 10⁻⁹ M, or higher, may be even more preferred.

Antibodies that are especially active at inhibiting tumour angiogenesis are preferred to anti-cancer therapeutic agents, and they can be selected for this activity using methods well known in the art.

Preferably, when the antibody is administered to an individual, the antibody binds to the target portion of MMRN2 with a greater affinity than for any other molecule in the individual. Preferably, the antibody binds to target portion of MMRN2 with at least 2, or at least 5, or at least 10 or at least 50 times greater affinity than for any other molecule in the individual. More preferably, the antibody binds to target portion of MMRN2 with at least 100, or at least 1,000, or at least 10,000 times greater affinity than any other molecule in the individual. Preferably, the antibody molecule selectively binds the portion of MMRN2 without significantly binding other polypeptides in the body.

A sixth aspect of the invention provides a nucleic acid molecule encoding an antibody as defined above in the fifth aspect of the invention. The nucleic acid molecule may be comprised in a vector, such as an expression vector.

The term “antibody” or “antibody molecule” as used herein includes but is not limited to polyclonal, monoclonal, chimeric, single chain, Fab fragments and fragments produced by a Fab expression library. Such fragments include fragments of whole antibodies which retain their binding activity for a target substance, Fv, F(ab′) and F(ab′)2 fragments, as well as single chain antibodies (scFv), fusion proteins and other synthetic proteins which comprise the antigen-binding site of the antibody. The term also includes antibody-like molecules which may be produced using phage-display techniques or other random selection techniques for molecules which bind to the specified polypeptide or to particular regions of it. Thus, the term antibody includes all molecules which contain a structure, preferably a peptide structure, which is part of the recognition site (i.e. the part of the antibody that binds or combines with the epitope or antigen) of a natural antibody. Furthermore, the antibodies and fragments thereof may be humanised antibodies, which are now well known in the art.

By “ScFv molecules” we mean molecules wherein the V_{H} and V_{L} partner domains are linked via a flexible oligopeptide. Engineered antibodies, such as ScFv antibodies, can be made using the techniques and approaches long known in the art. The advantages of using antibody fragments, rather than whole antibodies, are several-fold. The smaller size of the fragments may lead to improved pharmacological properties, such as better penetration to the target site. Effector functions of whole antibodies, such as complement binding, are removed. Fab, Fv, ScFv and dAb antibody fragments can all be expressed in and secreted from E. coli, thus allowing the facile production of large amounts of the fragments. Whole antibodies, and F(ab′)₂ fragments are “bivalent'. By “bivalent” we mean that the antibodies and F(ab′)₂ fragments have two antigen combining sites. In contrast, Fab, Fv, ScFv and dAb fragments are usually monovalent, having only one antigen combining site.

It is possible however that the ScFv may be monovalent, divalent, trivalent or tetravalent. The ScFv may be a diabody, tribody, or a tetrabody. The two or more V_{H} and V_{L} partner domains in a divalent, trivalent or tetravalent or diabody, tribody, or a tetrabody may be different. In such a situation, an ScFv agent may comprise more than 2 or more than 3, for example 4 different V_{H} and V_{L} domains.

Antibodies may be produced by standard techniques, for example by immunisation with the appropriate (glyco)polypeptide or portion(s) thereof, or by using a phage display library.

If polyclonal antibodies are desired, a selected mammal (e.g., mouse, rabbit, goat, horse, etc) is immunised with an immunogenic polypeptide bearing a desired epitope(s), optionally haptenised to another polypeptide. Depending on the host species, various adjuvants may be used to increase immunological response. Such adjuvants include, but are not limited to, Freund's, mineral gels such as aluminium hydroxide, and surface active substances such as lysolecithin, pluronic polyols, polyanions, peptides, oil emulsions, keyhole limpet to hemocyanin, and dinitrophenol. Serum from the immunised animal is collected and treated according to known procedures. If serum containing polyclonal antibodies to the desired epitope contains antibodies to other antigens, the polyclonal antibodies can be purified by immunoaffinity chromatography. Techniques for producing and processing polyclonal antisera are well known in the art.

Monoclonal antibodies directed against entire polypeptides or particular epitopes thereof can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced against the polypeptides listed above can be screened for various properties; i.e., for isotype and epitope affinity. Monoclonal antibodies may be prepared using any of the well-known techniques which provides for the production of antibody molecules by continuous cell lines in culture.

It is preferred if the antibody is a monoclonal antibody. In some circumstances, particularly if the antibody is to be administered repeatedly to a human patient, it is preferred if the monoclonal antibody is a human monoclonal antibody or a humanised monoclonal antibody, which are suitable for administration to humans without engendering an immune response by the human against the administered immunoglobulin. Suitably prepared non-human antibodies can be “humanised” in known ways, for example by inserting the CDR regions of mouse antibodies into the framework of human antibodies. Humanised antibodies can be made using the techniques and approaches described in Verhoeyen et al (1988) Science, 239, 1534-1536, and in Kettleborough eta!, (1991) Protein Engineering, 14(7), 773-783. In some instances, Fv framework residues of the human immunoglobulin are replaced by corresponding non-human residues. In general, the humanised antibody will contain variable domains in which all or most of the CDR regions correspond to those of a non-human immunoglobulin, and framework regions which are substantially or completely those of a human immunoglobulin consensus sequence.

Completely human antibodies may be produced using recombinant technologies. Typically large libraries comprising billions of different antibodies are used. In contrast to the previous technologies employing chimerisation or humanisation of e.g. murine antibodies this technology does not rely on immunisation of animals to generate the specific antibody. Instead the recombinant libraries comprise a huge number of pre-made antibody variants wherein it is likely that the library will have at least one antibody specific for any antigen. Thus, using such libraries, an existing antibody having the desired binding characteristics can be identified.

It is appreciated that when the antibody is for administration to a non-human individual, the antibody may have been specifically designed/produced for the intended recipient species.

WO 98/32845 and Soderlind et al (2000) Nature BioTechnol. 18: 852-856 describe technology for the generation of variability in antibody libraries. Antibody fragments derived from this library all have the same framework regions and only differ in their CDRs. Since the framework regions are of germline sequence the immunogenicity of antibodies derived from the library, or similar libraries produced using the same technology, are expected to be particularly low (Soderlind et al, 2000). This property is of great value for therapeutic antibodies, reducing the risk that the patient forms antibodies to the administered antibody, thereby reducing risks for allergic reactions, the occurrence of blocking antibodies, and allowing a long plasma half-life of the antibody. Thus, when developing therapeutic antibodies to be used in humans, modern recombinant library technology (Soderlind et al, 2001, Comb. Chem. & High Throughput Screen. 4: 409-416) is now used in preference to the earlier hybridoma technology.

By antibodies we also include heavy-chain antibodies structurally derived from camelidae antibodies, such as Nanobodies® (Ablynx). These are antibody-derived therapeutic proteins that contain the structural and functional properties of naturally-occurring heavy-chain antibodies. The Nanobody® technology was developed following the discovery that camelidae (camels and llamas) possess fully functional antibodies that lack light chains. These heavy-chain antibodies contain a single variable domain (VHH) and two constant domains (C_{H}2 and CH₃). The cloned and isolated VHH domain is a perfectly stable polypeptide harbouring the full antigen-binding capacity of the original heavy-chain antibody. These VHH domains with their unique structural and functional properties form the basis of Nanobodies®. They combine the advantages of conventional antibodies (high target specificity, high target affinity and low inherent toxicity) with important features of small molecule drugs (the ability to inhibit enzymes and access receptor clefts). Furthermore, they are stable, have the potential to be administered by means other than injection, are easier to manufacture, and can be humanised. (See, for example U.S. Pat. Nos. 5,840,526; 5,874,541; 6,005,079, 6.765,087; EP 1 589 107; WO 97/34103; WO97/49805; U.S. Pat. Nos. 5,800,988; 5,874, 541 and 6,015,695).

Compounds Comprising a Detectable Moiety

A seventh aspect of the invention provides a compound comprising a portion of MMRN2 according to the first or second aspect of the invention; and a detectable moiety. Preferences for the portion of MMRN2 include those described above in relation to the relevant aspects of this invention. Thus, the portion of MMRN2 may comprise the structure of Formula I (e.g. a peptide of no more than 180 amino acids comprising the structure of Formula I).

Such a compound can be used, in combination with an appropriate detection method, to detect the location of the compound in the individual, and hence to identify the sites and extent of angiogenesis (e.g. tumour angiogenesis) in the individual, as well as inhibition of angiogenesis (e.g. tumour angiogenesis) in the individual.

By a “detectable moiety” we include the meaning that the moiety is one which, when located at the target site following administration of the compound of the invention into a patient, may be detected, typically non-invasively from outside the body, and the site of the target located. Thus, the compounds of this aspect of the invention are useful in imaging and diagnosis, especially in the imaging and diagnosis of neovasculature of solid tumours, as is described further below.

Typically, the detectable moiety is or comprises a magnetic nano-particle, a radionuclide or a fluorophore.

Thus, in an embodiment, the detectable moiety may be a radioactive atom which is useful in imaging. Suitable radioactive atoms include technetium-99m or iodine-123 for scintigraphic studies. Others may be selected from the group consisting of: iodine-124; iodine-125; iodine-126; iodine-131; iodine-133; indium-111; indium-113m, fluorine-18; fluorine-19; carbon-11; carbon-13; copper-64; nitrogen-13; nitrogen-15; oxygen-15; oxygen-17; arsenic-72; gadolinium; manganese; iron; deuterium; tritium; yttrium-86; zirconium-89; bromine-77, gallium-67; gallium-68, ruthenium-95, ruthenium-97, ruthenium-103, ruthenium-105, mercury-107, rhenium-99m, rhenium-101, rhenium-105, scandium-47. Suitable methods for coupling such radioisotopes to the antibodies—either directly or via a chelating agent such as EDTA or DTPA—can be employed, as is known in the art.

Other readily detectable moieties include, for example, spin labels for magnetic resonance imaging (MRI) such as iodine-123 again, iodine-131, indium-111, fluorine-19, carbon-13, nitrogen-15, oxygen-17, gadolinium, manganese or iron. Clearly, the compound of the invention must have sufficient of the appropriate atomic isotopes in order for the molecule to be detectable.

The radio- or other label may be incorporated in the compound in known ways. For example, the portion of MMRN2 may be biosynthesised or synthesised by chemical amino acid synthesis using suitable amino acid precursors involving, for example, fluorine-19 in place of hydrogen. Labels such as ^{99m}TC, ¹²³I, ¹⁸⁶Rh, ¹⁸⁸Rh and ¹¹¹In can, for example, be attached via cysteine residues in the portion of MMRN2, or antibody thereto. Yttrium-90 can be attached via a lysine residue. The IODOGEN method (Fraker et al (1978) Biochem. Biophys. Res. Comm. 80, 49-57) can be used to incorporate iodine-123. The reference (“Monoclonal Antibodies in Immunoscintigraphy”, J. F. Chatal, CRC Press, 1989) describes other methods in detail.

Many suitable fluorophores and detection methods are well known in the art and are described, for example by Stefan Andersson-Engels et al (1997) “In vivo fluorescence imaging for tissue diagnostics. Phys. Med. Biol. 42: 815-824; Altinoğlu et al (2008) “Near-Infrared Emitting Fluorophore-Doped Calcium Phosphate Nanoparticles for In Vivo Imaging of Human Breast Cancer” ACS Nano 2(10): 2075-84; and Chin et al (2009) “In-vivo optical detection of cancer using chlorin e6—polyvinylpyrrolidone induced fluorescence imaging and spectroscopy” BMC Medical imaging 9:1 (doi:10.1186/1471-2342-9-1). Examples include fluorescein and its derivatives, fluorochrome, rhodamine and its derivatives, Green Fluorescent Protein (GFP), dansyl, umbelliferone etc. In such conjugates, the portions of MMRN2 of the invention or their functional fragments can be prepared by methods known to the person skilled in the art.

The detectable moiety may comprise a detectable enzyme such as peroxidase, alkaline phosphatase, beta-D-galactosidase, glucose oxidase, glucose amylase, carbonic anhydrase, acetylcholinesterase, lysozyme, malate dehydrogenase or glucose 6-phosphate dehydrogenase.

The detectable moiety may comprise a molecule such as biotin, digoxygenin or 5-bromodeoxyuridine.

The detectable moiety may comprise a chemiluminescent label such as luminol and the dioxetanes, or a bioluminescent label such as luciferase and luciferin.

The detectable label may comprise an affinity tag (e.g. histidine tag, Fc tag, BirA tag, maltose-binding protein tag, GST tag, HA tag, FLAG tag); or a directly detectable label (such as a fluorophore, a radioisotope, a contrast agent, or a luminescent label); or an indirectly detectable label (such as an enzyme, an enzyme substrate, an antibody, an antibody fragment, an antigen, a hapten, a ligand, an affinity molecule, a chromogenic substrate, a protein, a peptide, a nucleic acid, a carbohydrate and a lipid).

It will be appreciated that the detectable moiety may be one that is useful to detect the portion of MMRN2 during its manufacture and/or purification. Conveniently, the compound comprising the portion of MMRN2 and detectable moiety may also comprise a cleavage site, for example to enable removal of a detectable moiety during purification. Any suitable cleavage site known in the art may be used. An example is the tobacco etch virus (TEV) cleavage site.

An eighth aspect of the invention provides a polynucleotide encoding a compound as defined above in the seventh aspect of the invention, wherein the portion of MMRN2 and the detectable moiety are polypeptides which are fused. It will be appreciated that the eighth aspect of the invention also provides a vector comprising a polynucleotide encoding a compound as defined above in the seventh aspect of the invention, wherein the portion of MMRN2 and the detectable moiety are polypeptides which are fused.

Compounds Comprising a Cytotoxic Moiety

A ninth aspect of the invention provides a compound comprising: a portion of MMRN2 according to the first or second aspects of the invention; and a cytotoxic moiety. Preferences for the portion of MMRN2 include those described above in relation to the relevant aspects of the invention. Thus, the portion of MMRN2 may comprise the structure of Formula I (e.g. a peptide of no more than 180 amino acids comprising the structure of Formula I).

The cytotoxic moiety may be directly or indirectly toxic to cells in neovasculature or cells which are in close proximity to and associated with neovasculature. By “directly cytotoxic” we include the meaning that the moiety is one which on its own is cytotoxic. By “indirectly cytotoxic” we include the meaning that the moiety is one which, although is not itself cytotoxic, can induce cytotoxicity, for example by its action on a further molecule or by further action on it. For example, an indirect cytotoxic moiety may act to recruit an immune cell (e.g. a cytotoxic immune cell such as a cytotoxic T cell), and thereby indirectly induce a cytotoxic effect.

Typically, the cytotoxic moiety is selected from a directly cytotoxic chemotherapeutic agent, a directly cytotoxic polypeptide, a moiety which is able to convert a prodrug into a cytotoxic drug, a radiosensitizer, a directly cytotoxic nucleic acid, a nucleic acid molecule that encodes a directly or indirectly cytotoxic polypeptide or a radioactive atom. Examples of such cytotoxic moieties, as well as methods of making the conjugates comprising the cytotoxic moiety, are provided in our earlier publications WO 02/36771, WO 2004/046191, and WO 2011/027132 incorporated herein by reference. It will be appreciated that it may be necessary for the compound to comprise a translocation domain capable of translocating the cytotoxic moiety into the cell, where the cytotoxic moiety is a directly cytotoxic moiety which exerts its cytotoxic effect inside the cell. For example, the translocation domain is preferably capable of forming permeable pores in the cellular membrane. The domain may be a translocating domain of an enzyme, such as a bacterial toxin or viral protein.

In one embodiment the cytotoxic moiety is a cytotoxic chemotherapeutic agent. Cytotoxic chemotherapeutic agents, such as anticancer agents, are well known in the art, and include those described above.

Various of the cytotoxic moieties mentioned above, such as cytotoxic chemotherapeutic agents, have previously been attached to antibodies and other targeting agents, and so compounds of the invention comprising these agents may readily be made by the person skilled in the art. For example, carbodiimide conjugation (Bauminger & Wilchek (1980) Methods Enzymol. 70, 151-159) may be used to conjugate a variety of agents, including doxorubicin, to proteins. Other methods for conjugating a cytotoxic moiety to a protein can also be used. For example, sodium periodate oxidation followed by reductive alkylation of appropriate reactants can be used, as can glutaraldehyde cross-linking. Methods of cross-linking polypeptides are known in the art and described in WO 2004/046191. However, it is recognised that, regardless of which method of producing a compound of the invention is selected, a determination must be made that the portion of MMRN2 maintains its targeting ability (e.g. the ability to bind to CLEC14A and/or CD93) and that the attached moiety maintains its relevant function.

In a further embodiment of the invention, the cytotoxic moiety may be a cytotoxic peptide or polypeptide moiety by which we include any moiety which leads to cell death. Cytotoxic peptide and polypeptide moieties are well known in the art and include, for example, ricin, abrin, Pseudomonas exotoxin, tissue factor and the like. Methods for linking them to targeting moieties such as polypeptides are also known in the art, and include, for example, conventional ways of crosslinking polypeptides and production of the compound as a fusion polypeptide using recombinant DNA techniques. The use of ricin as a cytotoxic moiety is described in Burrows & Thorpe (1993) Proc. Natl. Acad. Sci. USA 90, 8996-9000, and the use of tissue factor, which leads to localised blood clotting and infarction of a tumour, has been described by Ran et al (1998) Cancer Res. 58, 4646-4653 and Huang et al (1997) Science 275, 547-550. Tsai et al (1995) Dis. Colon Rectum 38, 1067-1074 describes the abrin A chain conjugated to a monoclonal antibody. Other ribosome inactivating proteins are described as cytotoxic moieties in WO 96/06641. Pseudomonas exotoxin may also be used as the cytotoxic polypeptide moiety (Aiello et al (1995) Proc. Natl. Acad. Sci. USA 92, 10457-10461).

Certain cytokines, such as TNFα, INFγ and IL-2, may also be useful as cytotoxic moieties.

Certain radioactive atoms may also be cytotoxic if delivered in sufficient doses. Thus, the cytotoxic moiety may comprise a radioactive atom which, in use, delivers a sufficient quantity of radioactivity to the target site so as to be cytotoxic. Suitable radioactive atoms include phosphorus-32, iodine-125, iodine-131, indium-111, rhenium-186, rhenium-188 or yttrium-90, or any other isotope which emits enough energy to destroy neighbouring cells, organelles or nucleic acid. Preferably, the isotopes and density of radioactive atoms in the compound of the invention are such that a dose of more than 4000 cGy (preferably at least 6000, 8000 or 10000 cGy) is delivered to the target site and, preferably, to the cells at the target site and their organelles, particularly the nucleus.

The radioactive atom may be attached to the portion of MMRN2 in known ways. For example EDTA or another chelating agent may be attached to the portion and used to attach ¹¹¹In or ⁹⁰Y. Tyrosine residues may be labelled with ¹²⁵I or ¹³¹I.

The cytotoxic moiety may be a radiosensitizer. Radiosensitizers include fluoropyrimidines, thymidine analogues, hydroxyurea, gemcitabine, fludarabine, nicotinamide, halogenated pyrimidines, 3-aminobenzamide, 3-aminobenzodiamide, etanixadole, pimonidazole and misonidazole (see, for example, McGinn et al (1996) J. Natl. Cancer Inst. 88, 1193-11203; Shewach & Lawrence (1996) Invest. New Drugs 14, 257-263; Horsman (1995) Acta Oncol. 34, 571-587; Shenoy & Singh (1992) Clin. Invest. 10, 533-551; Mitchell et al (1989) Int. J. Radiat. Biol. 56, 827-836; Iliakis & Kurtzman (1989) Int. J. Radiat. Oncol. Biol. Phys. 16, 1235-1241; Brown (1989) Int. J. Radiat. Oncol. Blot Phys. 16, 987-993; Brown (1985) Cancer 55, 2222-2228).

The cytotoxic moiety may be a procoagulant factor, such as the extracellular domain of tissue factor (Rippmann et al (2000) “Fusion of the tissue factor extracellular domain to a tumour stroma specific single-chain fragment variable antibody results in an antigen-specific coagulation-promoting molecule.” Biochem J. 349: 805-12; Huang et a/ (1997) “Tumor infarction in mice by antibody-directed targeting of tissue factor to tumor vasculature.” Science. 275(5299): 547-550.

The cytotoxic moiety may be an indirectly cytotoxic polypeptide. In a particularly preferred embodiment, the indirectly cytotoxic polypeptide is a polypeptide which has enzymatic activity and can convert a relatively non-toxic prodrug into a cytotoxic drug. When the targeting moiety is an antibody, this type of system is often referred to as ADEPT (Antibody-Directed Enzyme Prodrug Therapy). The system requires that the targeting moiety locates the enzymatic portion to the desired site in the body of the patient (e.g. the site of new vascular tissue associated with a tumour) and after allowing time for the enzyme to localise at the site, administering a prodrug which is a substrate for the enzyme, the end product of the catalysis being a cytotoxic compound. The object of the approach is to maximise the concentration of drug at the desired site and to minimise the concentration of drug in normal tissues (Senter et al (1988) “Anti-tumor effects of antibody-alkaline phosphatase conjugates in combination with etoposide phosphate” Proc. Natl. Acad. Sci. USA 85, 4842-4846; Bagshawe (1987) Br. J. Cancer 56, 531-2; and Bagshawe, et al (1988) “A cytotoxic moiety can be generated selectively at cancer sites” Br. J. Cancer. 58, 700-703); Bagshawe (1995) Drug Dev. Res. 34, 220-230 and WO 2004/046191, describe various enzyme/prodrug combinations which may be suitable in the context of this invention.

Typically, the prodrug is relatively non-toxic compared to the cytotoxic drug. Typically, it has less than 10% of the toxicity, preferably less than 1% of the toxicity as measured in a suitable in vitro cytotoxicity test.

It is likely that the moiety which is able to convert a prodrug to a cytotoxic drug will be active in isolation from the rest of the compound but it is necessary only for it to be active when (a) it is in combination with the rest of the compound and (b) the compound is attached to, adjacent to or internalised in target cells.

The cytotoxic moiety may be one which becomes cytotoxic, or releases a cytotoxic moiety, upon irradiation. For example, the boron-10 isotope, when appropriately irradiated, releases α particles which are cytotoxic (U.S. Pat. No. 4,348,376; Primus et al (1996) Bioconjug. Chem. 7: 532-535).

Similarly, the cytotoxic moiety may be one which is useful in photodynamic therapy such as photofrin (see, for example, Dougherty et al (1998) J. Natl. Cancer Inst. 90, 889-905).

In a particular embodiment, the cytotoxic moiety is an antibody, such as one that specifically binds to an immune cell, such as a cytotoxic immune cell (e.g. T cell). Thus, it will be appreciated that the compound of the invention may be useful in targeting an immune synapse, which is considered to be an interface between a target cell (e.g. a cell overexpressing CLE14 or CD93) and a lymphocyte such as an effector T cell or Natural Killer cell. In this way, the compounds of the invention are similar to bispecific T cell engagers (BiTEs), which are well known in the art.

In another embodiment, the cytotoxic moiety is a pyrrolobenzodiazepine dimer (PBD). PBDs are potent anticancer agents which have been shown to have broad spectrum anti-tumour activity in vivo. These drugs exert their activity by binding the minor groove of DNA and linking the two DNA strands together in a way that cells find difficult to recognise and repair. Thus the compound of the invention may comprise a PBD. Further information on PBDs can be found in Hartley et al, 2012 (Invest New Drugs 30: 950-958).

A tenth aspect of the invention provides a polynucleotide encoding a compound as defined above in the ninth aspect of the invention, wherein the portion of MMRN2, and the cytotoxic moiety are polypeptides which are fused. The aspect also includes a vector comprising such a polynucleotide.

Chimeric Antigen Receptors (CARs)

An eleventh aspect of the invention provides a chimeric antigen receptor (CAR) comprising (a) a portion of MMRN2 or a variant thereof that binds to CLEC14A and/or CD93 (b) a transmembrane domain; and (c) an intracellular signalling domain.

CARs are recombinant receptors for antigen, which, in a single molecule, redirect the specificity and function of T lymphocytes and other immune cells (Sadelain et al 2013 Cancer Discov 3(4): 388). The general premise for their use in cancer immunotherapy is to rapidly generate tumour-targeted T cells, bypassing the barriers and incremental kinetics of active immunisation. Once expressed in T cells, CAR-modified T cells acquire supra-physiological properties and act as “living drugs” that may exert both immediate and long- term effects. The engineering of CARs into T cells requires that T cells be cultured to allow for transduction and expansion. The transduction may utilise a variety of methods, but stable gene transfer is required to enable sustained CAR expression in clonally expanding and persisting T cells. In principle, any cell surface molecule can be targeted through a CAR, thus overriding tolerance to self-antigens and the antigen recognition gaps in the physiological T cell repertoire that limit the scope of T cell reactivity. Various T cell subsets, as well as T cell progenitors and other immune cells such as natural killer cells, can be targeted to any unwanted cell by modification with a CAR.

By a portion of MMRN2 or a variant thereof that binds to CLEC14A and/or CD93, we include the meaning of any portion of the full length native MMRN2 polypeptide or any portion of a variant of the full length native MMRN2 polypeptide, provided that that portion binds to CLEC14A and/or CD93. Preferences for the variant of native MMRN2, for the length of the portion, and for the degree of sequence identity between the portion and corresponding portion of wild type MMRN2 include those described above in relation to the first aspect of the invention. It is preferred if the portion of MMRN2 selectively binds to CLEC14A and/or CD93.

In a particularly preferred embodiment, the portion of MMRN2 contained within the CAR is the portion of MMRN2 of the first or second aspect of the invention. Thus, the portion of MMRN2 may comprise the structure of Formula I (e.g. a peptide of no more than 180 amino acids comprising the structure of Formula I). It will be appreciated that the portion of MMRN2 may need to be attached to a linker or spacer moiety to allow it to bind to CLEC14A and/or CD93. Such linker or spacer moieties are common in CARs that comprise scFv antibodies, and so are well known to the skilled person. Typically, the portion of MMRN2 is attached to a linker peptide that adopts a random coil conformation, such as peptides that contain alanine or proline or a mixture of alanine plus proline residues. Other possible linkers include glycine and/or serine residues. The linker peptide may be between 2 and 100 amino acids, such as 2 and 50 amino acids.

By a transmembrane domain we include the meaning of any moiety that is capable of being embedded in a lipid membrane. By being embedded in a lipid membrane we include the meaning of the transmembrane domain favourably interacting with the hydrophobic portions of the lipids that make up the lipid membrane. Insertion into lipid membranes may be assayed using any suitable method known in the art, including fluorescence labelling with fluorescence microscopy. Hence, it will be appreciated that the transmembrane domain is one that locates the CAR molecule within the lipid membrane.

In an embodiment, the transmembrane domain comprises the transmembrane domain of a protein (e.g. a transmembrane protein). The transmembrane domain can include one or more additional amino acids adjacent to the transmembrane region, such as one or more amino acids associated with the extracellular region of the protein from which the transmembrane domain was derived (e.g. 1, 2, 3, 4, 5, 6, 7, 8, 9, 10 up to 15 amino acids of the extracellular region) and/or one or more additional amino acids associated with the intracellular region of the protein from which the transmembrane protein is derived (e.g. 1, 2, 3, 4, 5, 6, 7, 8, 9, 10 up to 15 amino acids of the intracellular region). In an embodiment, the transmembrane domain is one that is associated with one of the other domains of the CAR. In an embodiment, the transmembrane domain comprises the transmembrane portion of an intracellular signalling protein that constitutes at least part of the intracellular signalling domain. In some instances, the transmembrane domain can be selected or modified by amino acid substitution to avoid binding of such domains to the transmembrane domains of the same or different surface membrane proteins, e.g. to minimise interactions with other members of the receptor complex. In some instances, the transmembrane domain is capable of homodimerisation with another CAR on the cell surface.

The transmembrane domain may be derived either from a natural or from a recombinant source. The domain may be derived from any membrane-bound or transmembrane protein. In one embodiment, the transmembrane domain is capable of signalling to the intracellular domain(s) whenever the CAR has bound to a target. A suitable transmembrane domain for use in the invention may include the transmembrane region(s) of the alpha, beta or zeta chain of the T cell receptor, CD28, CD3 epsilon, CD8, CD45 and CD4.

In some instances, the transmembrane domain can be attached to the portion of MMRN2 that binds to CLEC14A, optionally via a hinge region. The hinge region may comprise one or more immunoglobulin domains. Particular examples include the Fc region of IgG1 and the immunoglobulin-like extracellular regions of CD4 and CD8. The hinge may be from a human protein such as human immunoglobulin.

Typically, the transmembrane domain comprises predominantly hydrophobic amino acid residues such as leucine and valine.

In an embodiment, a short oligo- or polypeptide linker, such as between 2 and 10 amino acids in length, may form the linkage between the transmembrane domain and the intracellular signalling domain of the CAR. The linker may comprise glycine and/or serine residues (eg a glycine-serine doublet).

By intracellular signalling domain we include the meaning of a domain that is capable of activating at least one of the normal functions of the cell in which the CAR is introduced, such as at least one of the normal effector functions of an immune cell (e.g. T cell). An effector function refers to a specialised function of a cell. The effector function of a T cell, for example, may be cytolytic function or helper activity including the secretion of cytokines. Thus, the intracellular signalling domain may be a portion of a protein which transduces the effector function signal and directs the cell (e.g. T cell) to perform a specialised function.

Generally, the whole intracellular signalling domain can be used; however, it is appreciated that it is not necessary to use the entire domain, provided that whatever part of the signalling domain that is used is still capable of transducing the effector function signal. It will also be appreciated that variants of such intracellular signalling domains with substantially the same or greater functional capability may also be used. By this we include the meaning that the variants should have substantially the same or greater transduction of the effector functional signal. Typically, substantially the same or greater signal transduction includes at least 80%, 85%, 90%, 95%, 100%, 105%, 110%, 115%, or 120%, or more of the signal transduction of the unmodified intracellular signalling domain, wherein signal transduction of the unmodified intracellular signalling domain corresponds to 100%.

Methods for assessing transduction of effector function signal are well known to those skilled in the art and include, for example, assessing the amounts and/or activity of molecules (e.g. proteins such as cytokines) that are indicative of the transduced signal. Thus, when the signal is the cytolytic function of a T-cell, the methods may involve measurement of one or more cytokines secreted by the T-cell, which cytokines are known to have a cytolytic activity (e.g. IFN gamma). Another means of assessing the cytolytic function is by a chromium release assay as is well known in the art.

Examples of intracellular signalling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are generally insufficient for full activation of a T cell and that a secondary and/or costimulatory signal may also be required. Thus, T cell activation can be said to be mediated by two distinct classes of intracellular signaling sequences: those that initiate antigen-dependent primary activation through the TCR (primary intracellular signaling domains) and those that act in an antigen-independent manner to provide a secondary or costimulatory signal (secondary intracellular signalling domain, such as a costimulatory domain). Costimulatory domains promote activation of effector functions and may also promote persistence of the effector function and/or survival of the cell.

A primary intracellular signaling domain regulates primary activation of the TCR complex either in a stimulatory way, or in an inhibitory way. Primary intracellular signaling domains that act in a stimulatory manner may contain signalling motifs which are known as immunoreceptor tyrosine-based activation motifs or ITAMs (e.g. 2, 3, 4, 5 or more ITAMs). Thus, the intracellular signalling domain may comprise one or more ITAMs. Examples of ITAM containing primary intracellular signaling domains that are of particular use in the invention include those of CD3 zeta, Fc receptor gamma, Fc receptor beta, CD3 gamma, CD3 delta , CD3 epsilon, CD5, CD22, CD79a, CD79b, and CD66d.

In one embodiment, a CAR of the invention comprises an intracellular signaling domain of CD3-zeta.

It will be appreciated that one or more ITAMs of the intracellular signalling domain may be modified, for example by mutation. The modification may be used to increase or decrease the signalling function of the ITAM as compared to the native ITAM domain.

As mentioned above, the intracellular signalling domain may comprise a primary intracellular signalling domain by itself, or it may comprise a primary intracellular signalling domain in combination with one or more secondary intracellular signalling domains, such as one or more costimulatory signalling domains. Thus, the intracellular signalling domain of the CAR may comprise the CD3 zeta signalling domain by itself or in combination with one or more other intracellular signalling domains such as one or more costimulatory signalling domains.

The costimulatory signaling domain refers to a portion of the CAR comprising the intracellular domain of a costimulatory molecule. A costimulatory molecule may be a cell surface molecule other than an antigen receptor or its ligands that is required for an efficient response of immune cells (e.g. lymphocytes) to an antigen. Examples of such molecules include CD28, 4-1BB (CD137), OX40, ICOS, DAP10, CD27, CD30, CD40, ICOS, lymphocyte function-associated antigen-1 (LFA-1), CD2, CD7, LIGHT, NKG2C, B7-H3, and a ligand that specifically binds with CD83, and the like. For example, CD27 co-stimulation has been demonstrated to enhance expansion, effector function, and survival of human CART cells in vitro and augments human T cell persistence and anti-tumour activity in vivo (Song et al. Blood. 2012; 119(3):696-706).

The intracellular signaling sequences within the intracellular portion of the CAR of the invention may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences. In one embodiment, a glycine- serine doublet can be used as a suitable linker. In another embodiment, a single amino acid, such as an alanine or a glycine, can be used as a suitable linker.

In one embodiment, the intracellular signaling domain is designed to comprise two or more, for example 3, 4, 5, or more, costimulatory signalling domains. In an embodiment, the two or more, e.g., 2, 3, 4, 5, or more, costimulatory signaling domains, are separated by a linker molecule, such as one described herein. In one embodiment, the intracellular signaling domain comprises two costimulatory signaling domains. In some embodiments, the linker molecule is a glycine residue. In some embodiments, the linker is an alanine residue.

In a preferred embodiment, the intracellular portion of the CAR comprises the signalling domain of CD3 zeta and the signalling domain of CD28.

In another embodiment, the intracellular portion of the CAR comprises the signaling domain of CD3-zeta and the signaling domain of 4-1BB.

In another embodiment, the intracellular portion of the CAR comprises the signaling domain of CD3-zeta and the signaling domain of OX40.

In another embodiment, the intracellular portion of the CAR comprises the signaling domain of CD3-zeta and the signaling domain of ICOS.

In another embodiment, the intracellular portion of the CAR comprises the signaling domain of CD3-zeta and the signaling domain of DAP10.

In another embodiment, the intracellular portion of the CAR comprises the signalling domain of CD3-zeta, the signalling domain of 4-1BB and the signalling domain of OX40.

In another embodiment, the intracellular portion of the CAR comprises the signaling domain of 4-1BB and the signaling domain of CD28.

In another embodiment, the intracellular portion of the CAR comprises the signaling domain of CD3-zeta, the signaling domain of 4-1BB and the signalling domain of CD28.

In an embodiment, the CAR further comprises a leader sequence. By a “leader sequence” we include the meaning of a peptide sequence that directs the CAR to the cell membrane. Thus, when the CAR is a chimeric fusion protein, it may contain a leader sequence at the amino-terminus (N-ter) of the portion of MMRN2 that binds CLEC14A. Optionally, the leader sequence is cleaved from the portion of MMRN2 during cellular processing and localisation of the CAR to the cellular membrane.

An exemplary leader sequence is the oncostatin M leader sequence MGVLLTQRTLLSLVLALLFPSMAS (SEQ ID NO: 4) or a variant thereof (e.g. one having at least 70%, 75%, 80%, 85%, 90%, 95% or 99% sequence identity to said sequence); however, other leader sequences will also be known to a person skilled in the art, such as the leader sequence from CD8. The core of a leader sequence typically contains a stretch of hydrophobic amino acids that has a tendency to form a single alpha-helix.

In a further embodiment, the CAR may comprise a suicide moiety. By a suicide moiety we include the meaning of a molecule which possesses an inducible capacity to lead to the death of the cell in whose cellular membrane the CAR resides (e.g. T cell). In this way, the effect that the CARs have on a subject can be tightly controlled via selective deletion of the cells that comprise them. Conveniently, the suicide moiety comprises the epitope of an antibody that is either directly or indirectly cytotoxic. Antibodies that are directly cytotoxic include lytic antibodies such as Rituximab, which binds to CD20. Thus, in one embodiment, the CAR may comprise a CD20 epitope. Antibodies may also be indirectly cytotoxic by being conjugated to one or more cytotoxic moieties. Suicide moieties are well known in the art and are reviewed in Jones et al (Front Pharmacol 2014, 5: 254). An example is inducible caspase-9.

In an embodiment, the CAR comprises a portion of MMRN2 of the first or second aspects of the invention, a transmembrane domain, and an intracellular signalling domain (e.g. an intracellular signalling domain comprising a primary signalling domain such as CD3 zeta, and optionally one or more costimulatory domains such as CD28, 4-1BB, OX40, ICOS and DAP10).

In an embodiment, the CAR comprises a leader sequence (e.g. oncostatin M leader sequence), a portion of MMRN2 of the first or second aspects of the invention, a hinge region, a transmembrane domain, and an intracellular signalling domain (e.g. an intracellular signalling domain comprising a primary signalling domain such as CD3 zeta, and optionally one or more costimulatory domains such as CD28, 4-1BB, OX40, ICOS and DAP10).

A twelfth aspect of the invention provides a polynucleotide encoding a CAR as defined above in relation to the eleventh aspect of the invention. It will be appreciated that the twelfth aspect of the invention also provides a vector comprising a polynucleotide encoding a CAR defined above.

Polynucleotides, Vectors, Expression and Cells

The nucleic acid molecule of any of the fourth, sixth, eighth, tenth and twelfth aspects of the invention may be DNA or RNA, and is preferably DNA, in particular circumstances. In other circumstances, e.g. when employing cell therapy, RNA may be preferred. It may comprise deoxyribonucleotides, ribonucleotides, modified nucleotides or bases, and/or their analogues, or any substrate that can be incorporated into a polymer by DNA or RNA polymerase, or by a synthetic reaction. A polynucleotide may comprise modified nucleotides, such as methylated nucleotides and their analogues. If present, modification to the nucleotide structure may be imparted before or after assembly of the polymer. The sequence of nucleotides may be interrupted by non-nucleotide components. Suitable sequences can readily be determined based upon the knowledge of the genetic code.

A thirteenth aspect of the invention provides a vector comprising the polynucleotide of any of the fourth, sixth, eighth, tenth and twelfth aspects of the invention.

A fourteenth aspect of the invention provides a host cell comprising a polynucleotide according to any of the fourth, sixth, eighth, tenth and twelfth aspects of the invention, and/or a MMRN2 portion according to the first or second aspects of the invention, or an antibody according to the fifth aspect of the invention, or a CAR according to the eleventh aspect of the invention, or a vector according to the thirteenth aspect of the invention.

The vector can be of any type, and refers to a nucleic acid that comprises an isolated nucleic acid and which can be used to deliver the isolated nucleic acid to the interior of a cell. Numerous vectors are known in the art including linear polynucleotides, polynucleotides associated with ionic or amphiphilic compounds, plasmids, and viruses. Thus, the term vector includes an autonomously replicating plasmid or a virus. The term should also be construed to further include non-plasmid and non-viral compounds which facilitate transfer of nucleic acid into cells, such as, for example, a polylysine compound, liposome, and the like.

Conveniently, the vector is an expression vector. Expression vectors contain elements (e.g., promoter, signals of initiation and termination of translation, as well as appropriate regions of regulation of transcription) which allow the expression and/or the secretion of products in a host cell. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide. Typical cloning vectors contain transcription and translation terminators, initiation sequences, and promoters useful for regulation of the expression of the desired nucleic acid sequence.

The vector can be suitable for replication and integration in eukaryotes, and/or it may be suitable for expression in prokaryotes, such as in bacterial species. Preferably, the vector is capable of expressing the nucleic acids of the invention in mammalian cells (e.g. human cells), such as mammalian (e.g. human) immune cells (e.g. T cells), for example for the expression of CARs.

The nucleic acids of the invention can also be cloned into a number of types of vectors including a plasmid, a phagemid, a phage derivative, an animal virus, and a cosmid. Vectors of particular interest include expression vectors, replication vectors, probe generation vectors, and sequencing vectors.

Any of a variety of host cells can be used, such as a prokaryotic cell, for example, E. coli, or a eukaryotic cell, for example a mammalian cell such as Chinese Hamster Ovary (CHO) cell, or a yeast, insect or plant cell. Many suitable vectors and host cells are very well known in the art. Preferably, the host cell is a stable cell line. Alternatively, the host cell may be a cell obtained from a patient, e.g. a T cell or other immune cell, as discussed further below.

The invention also includes methods for making a portion of MMRN2, a fusion protein, an antibody that selectively binds to the MMRN2 portion, or a CAR according to the invention. For example, the invention comprises expressing in a suitable host cell a recombinant vector encoding the portion of MMRN2, a fusion protein, an antibody that selectively binds to the MMRN2 portion, or a CAR, and recovering the product. Methods for expressing and purifying polypeptides are very well known in the art.

The invention also provides a method of producing a cell comprising introducing a polynucleotide molecule according to the fourth, sixth, eighth, tenth and twelfth aspects of the invention, or a vector according to the thirteenth aspect of the invention. Suitable methods of introducing polynucleotide molecules and/or vectors include those described above, and are generally known in the art. Particularly, electroporation may be used.

Any of a variety of host cells can be used, such as a prokaryotic cell, for example, E. coli, or a eukaryotic cell, for example a mammalian cell such as Chinese Hamster Ovary (CHO) cell, or a yeast, insect or plant cell.

In addition to a host cell being used in a method to produce a portion of MMRN2, a fusion protein, an antibody of the invention, or a CAR of the invention, the host cell itself may be used directly in therapy, for example in cell mediated therapy. Thus, the invention provides a method of treatment, comprising administering a host cell according to the invention to the subject, for example for use in medicine or for combating cancer and/or for inhibiting angiogenesis. Accordingly, the invention also provides a host cell comprising a polynucleotide molecule according to the fourth, sixth, eighth, tenth and twelfth aspects of the invention, e.g. an RNA molecule, or a vector according to the thirteenth aspect of the invention, e.g. a gamma retrovirus or a lentivirus, for use in medicine, for example for use in the treatment of cancer. The invention also provides for the use of said host cell in the preparation of a medicament for use in medicine, for example for use in the treatment of cancer. Preferences for the portion of MMRN2, antibody, CAR, and fusion protein are as outlined above.

In a preferred embodiment, the host cell is a mammalian cell (e.g. a human cell).

In a further preferred embodiment, the host cell is an immune cell, preferably a mammalian immune cell such as a human immune cell. This is particularly relevant when the host cell of the invention expresses a CAR of the invention. Immune cells include T cells and natural killer (NK) cells. The T cell may be any of an alpha-beta T cell, a gamma-delta T cell, a memory T cell (e.g. a memory T cell with stem cell-like properties). The NK cell may be an invariant NK cell.

In a particularly preferred embodiment, the immune cell is a memory T cell with stem cell like properties.

The cell may be “autologous” or “allogeneic”, as described further below.

Immune cells such as T cells can be obtained from a number of sources peripheral blood mononuclear cells, bone marrow, lymph node tissue, cord blood, thymus tissue, tissue from a site of infection, ascites, pleural effusion, spleen tissue, and tumors. Any number of cell lines (e.g. immune cell lines such as T cell lines) available in the art, may also be used.

In an embodiment, immune cells (e.g. T cells) are obtained from a unit of blood collected from a subject using any suitable techniques known in the art such as Ficoll™ separation. In another embodiment, cells from the circulating blood of a subject are obtained by apheresis. The apheresis product typically contains lymphocytes, including T cells, monocytes, granulocytes, B cells, other nucleated white blood cells, red blood cells, and platelets. It will be appreciated that the cells collected by apheresis may be washed to remove the plasma fraction and to place the cells in an appropriate buffer or media for subsequent processing steps. For example, the cells may be washed with phosphate buffered saline (PBS). Alternatively, the wash solution lacks calcium and may lack magnesium or may lack many if not all divalent cations. Initial activation steps in the absence of calcium can lead to magnified activation. A washing step may be accomplished by methods known to those in the art, such as by using a semi- automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In an embodiment, T cells are isolated from peripheral blood lymphocytes by lysing the red blood cells and depleting the monocytes, for example, by centrifugation through a PERCOLL™ gradient or by counter-flow centrifugal elutriation. Specific subpopulations of T cells, such as CD3+, CD28+, CD4+, CD8+, CD45RA+, and CD45RO+T cells, may be further isolated by positive or negative selection techniques known in the art. For example, T cells may be isolated by incubation with anti-CD3/anti-CD28 (e.g., 3×28)-conjugated beads, such as DYNABEADS® M-450 CD3/CD28 T, for a time period sufficient for positive selection of the desired T cells. Additionally or alternatively, a population of T cells may be enriched by negative selection, for instance by a combination of antibodies directed to surface markers unique to the negatively selected cells. Cell sorting and/or selection via negative magnetic immunoadherence or flow cytometry may be used.

It will be understood that cells derived from subjects that are to be modified to express the CAR of the invention may be stored for a period of time prior to their use (see, for example, therapeutic methods below). For example, the cells may be frozen, optionally after they have been washed, or they may be incubated under suitable conditions for them to remain viable until needed (e.g. on a rotator at 2-10° C. or at room temperature). In this way, the cells can be stored until such time as they might be needed. They may be stored in an unmodified state (i.e. wherein they do not express the CAR of the invention) or in a modified state (i.e. wherein they have been modified to express the CAR of the invention).

Prior to use in the therapeutic applications described further below, the cells may be activated and expanded generally using methods known in the art. For example, T cells may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9): 13191328, 1999; Garland et al., J. Immunol Meth. 227(1- 2):53-63, 1999).

T cells that have been exposed to varied stimulation times may exhibit different characteristics. For example, typical blood or apherised peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Particularly, T cells may be expanded prior to transduction with a polynucleotide or vector of the invention.

In an embodiment, the cell that expresses a portion of MMRN2 or antibody of the invention or CAR of the invention is further modified to comprise or express one or more other agents that enhance the activity of the cell (e.g. T cell) expressing the portion of MMRN or antibody of the invention or CAR.

For example, the other agent may be an agent that inhibits an inhibitory molecule that is known to decrease the ability of the CAR-expressing cell to mount an effective immune response. Examples of inhibitory molecules include PD1, PD-L1, CTLA4, TIM3, LAG3, VISTA, BTLA, TIGIT, LAIR1, CD160, 2B4 and TGFR beta. The agent that inhibits the inhibitory molecule may comprise a first polypeptide, e.g. an inhibitory molecule, associated with a second polypeptide that provides a positive signal to the cell, e.g. an intracellular signalling domain described herein.

Additionally or alternatively, the other agent may be a pro-inflammatory or pro-proliferative cytokine. The purpose of such cytokines may be to provide autocrine support to enhance the function, proliferation and/or persistence of antibody-expressing cells, and/or favourably alter the tumour microenvironment and recruit endogenous innate and cognate immune effects.

Medical Uses

The inventors have identified new molecular interactions that underline novel therapeutic approaches for inhibiting angiogenesis and/or combating cancer. In particular, all of an MMRN2 portion according to the first aspect of the invention, an agent according to the second aspect of the invention (which itself may be a portion of MMRN2), a fusion protein according to the third aspect of the invention, an antibody according to the fifth aspect of the invention, a polynucleotide according to any of the fourth, sixth, eighth, tenth or twelfth aspects of the invention, a vector according to the thirteenth aspect of the invention, a cell lo according to the fourteenth aspect of the invention, a compound according to the seventh or ninth aspects of the invention, a CAR according to the eleventh aspect of the invention, have therapeutic potential. These molecules are collectively referred to herein as the agent of the invention.

Accordingly, a fifteenth aspect of the invention provides the agent of the invention (ie an MMRN2 portion according to the first aspect of the invention, an agent according to the second aspect of the invention), a fusion protein according to the third aspect of the invention, an antibody according to the fifth aspect of the invention, a polynucleotide according to any of the fourth, sixth, eighth, tenth or twelfth aspects of the invention, a vector according to the thirteenth aspect of the invention, a cell according to the fourteenth aspect of the invention, a compound according to the seventh or ninth aspects of the invention, a CAR according to the eleventh aspect of the invention) for use in medicine.

A sixteenth aspect of the invention provides a method of inhibiting angiogenesis in an individual, the method comprising administering to the individual the agent of the invention (ie an MMRN2 portion according to the first aspect of the invention, an agent according to the second aspect of the invention, a fusion protein according to the third aspect of the invention, an antibody according to the fifth aspect of the invention, a polynucleotide according to any of the fourth, sixth, eighth, tenth or twelfth aspects of the invention, a vector according to the thirteenth aspect of the invention, a cell according to the fourteenth aspect of the invention, a compound according to the seventh or ninth aspects of the invention, or a CAR according to the eleventh aspect of the invention).

This aspect of the invention includes the agent of the invention (i.e. an MMRN2 portion according to the first aspect of the invention, an agent according to the second aspect of the invention, a fusion protein according to the third aspect of the invention, an antibody according to the fifth aspect of the invention, a polynucleotide according to any of the fourth, sixth, eighth, tenth or twelfth aspects of the invention, a vector according to the thirteenth aspect of the invention, a cell according to the fourteenth aspect of the invention, a compound according to the seventh or ninth aspects of the invention, or a CAR according to the eleventh aspect of the invention) for use in inhibiting angiogenesis in an individual.

The aspect of the invention also includes the use of the agent of the invention (i.e. an MMRN2 portion according to the first aspect of the invention, an agent according to the second aspect of the invention, a fusion protein according to the third aspect of the invention, an antibody according to the fifth aspect of the invention, a polynucleotide according to any of the fourth, sixth, eighth, tenth or twelfth aspects of the invention, a vector according to the thirteenth aspect of the invention, a cell according to the fourteenth aspect of the invention, a compound according to the seventh or ninth aspects of the invention, or a CAR according to the eleventh aspect of the invention) in the preparation of a medicament for inhibiting angiogenesis in an individual.

For the avoidance of doubt, it will also be appreciated that the invention also includes an in vitro or ex vivo method of inhibiting angiogenesis (e.g. tumour angiogenesis) comprising administering to tissue or cells in vitro or ex vivo the agent of the invention (i.e. an MMRN2 portion according to the first aspect of the invention, an agent according to the second aspect of the invention, a fusion protein according to the third aspect of the invention, an antibody according to the fifth aspect of the invention, a polynucleotide according to any of the fourth, sixth, eighth, tenth or twelfth aspects of the invention, a vector according to the thirteenth aspect of the invention, a cell according to the fourteenth aspect of the invention, a compound according to the seventh or ninth aspects of the invention, or a CAR according to the eleventh aspect of the invention). Preferably, the agent is the MMRN2 portion of the first aspect of the invention, an agent according to the second aspect of the invention, or the antibody of the fifth aspect of the invention, preferences for which includes those described above. The cells may be established cell lines, or cells that have been removed from an individual. The tissue or cells are preferably mammalian tissue or cells (e.g. endothelial tissue or cells), and most preferably are human tissue or cells. When the method is an ex vivo method, the agent may be administered to an angiogenesis model ex vivo. Suitable angiogenesis assays include assays for endothelial cell proliferation, migration and invasion, sponge assays and aortic ring assays. Further angiogenesis assays are described herein and in the Examples.

By “inhibiting angiogenesis” we include the meaning of reducing the rate or level of angiogenesis. The reduction can be a low level reduction of about 10%, or about 20%, or about 30%, or about 40% of the rate or level of angiogenesis. Preferably, the reduction is a medium level reduction of about 50%, or about 60%, or about 70%, or about 80% reduction of the rate or level of angiogenesis. More preferably, the reduction is a high level reduction of about 90%, or about 95%, or about 99%, or about 99.9% of the rate or level of angiogenesis. Most preferably, inhibition can also include the elimination of angiogenesis or its reduction to an undetectable level. Methods and assays for determining the rate or level of angiogenesis, and hence for determining whether and to what extent an agent inhibits angiogenesis, are known in the art and are described in further detail herein, including in the Examples.

Typically, the angiogenesis that is inhibited is tumour angiogenesis. Thus, the individual may have a solid tumour, which can be treated by inhibiting tumour angiogenesis, ie the solid tumour is associated with new blood vessel production. The term “tumour” is to be understood as referring to all forms of neoplastic cell growth including, but not limited to, tumours of the breast, ovary, liver, bladder, prostate, kidney, pancreas, stomach, oesophagus, lung and thyroid. Particularly, angiogenesis of a lung or pancreatic tumour may be inhibited.

Typically, the tumour is associated with undesirable neovasculature formation and agent reduces this to a useful extent. The reduction of undesirable neovasculature formation may halt the progression of the tumour and can lead to a clinically useful reduction of tumour size and growth, e.g. a reduction in tumour size or growth rate of at least 5, 10, 15, 20, 25, 30, 40, 50, 60, 70, 80 or 90%. Thus, the inhibition of tumour angiogenesis can be used to treat the tumour, for example, to prevent the (further) growth of the tumour, to prevent the spread of the tumour (metastasis), or to reduce the size of the tumour. The size of a tumour can be measured by imaging the tumour e.g. using an appropriate antibody specific for the tumour being targeted. Methods of tumour imaging are well known in the art. The growth rate of a tumour can be determined by measuring tumour size over a time period (e.g. before and after treatment, to determine whether treatment results in a reduction in the growth rate).

Preferably, the methods and medicaments of the invention are used to treat humans, in which case the portion of MMRN2 is a portion of human MMRN2. It is appreciated, however, that when the methods and medicaments of the invention are for treatment of non-human mammals, it is preferred if the portion of MMRN2 is one that is a portion of MMRN2 from the other species. Similarly, where the medicament is an agent which inhibits the interaction between MMRN2 and CD93, it is preferred if the agent is one that inhibits the interaction between human MMRN2 and human CD93, when used to treat a human, and so on.

Inhibition of angiogenesis may be useful in combating any disease or condition involving unwanted, undesirable or inappropriate angiogenesis. Such conditions include tumours/cancer, psoriasis, menorrhagia, endometriosis, arthritis (both inflammatory and rheumatoid), macular degeneration, Paget's disease, retinopathy and its vascular complications (including proliferative and of prematurity, and diabetic retinopathy), benign vascular proliferations, fibroses, obesity and inflammation.

Hence, a seventeenth aspect of the invention provides a method of combating a disease or condition in an individual, selected from the group consisting of cancer, psoriasis, menorrhagia, endometriosis, arthritis (both inflammatory and rheumatoid), macular degeneration, Paget's disease, retinopathy and its vascular complications (including proliferative and of prematurity, and diabetic retinopathy), benign vascular proliferations, fibroses, obesity and inflammation, the method comprising administering to the individual an MMRN2 portion according to the first aspect of the invention, an agent according to the second aspect of the invention, a fusion protein according to the third aspect of the invention, an antibody according to the fifth aspect of the invention, a polynucleotide according to any of the fourth, sixth, eighth, tenth or twelfth aspects of the invention, a vector according to the thirteenth aspect of the invention, a cell according to the fourteenth aspect of the invention, a compound according to the seventh or ninth aspects of the invention, or a CAR according to the eleventh aspect of the invention.

This aspect of the invention includes an MMRN2 portion according to the first aspect of the invention, an agent according to the second aspect of the invention, a fusion protein according to the third aspect of the invention, an antibody according to the fifth aspect of the invention, a polynucleotide according to any of the fourth, sixth, eighth, tenth or twelfth aspects of the invention, a vector according to the thirteenth aspect of the invention, a cell according to the fourteenth aspect of the invention, a compound according to the seventh or ninth aspects of the invention, or a CAR according to the eleventh aspect of the invention, for use in combating a disease or condition in an individual, selected from the group consisting of cancer, psoriasis, menorrhagia, endometriosis, arthritis (both inflammatory and rheumatoid), macular degeneration, Paget's disease, retinopathy and its vascular complications (including proliferative and of prematurity, and diabetic retinopathy), benign vascular proliferations, fibroses, obesity and inflammation.

The aspect of the invention also includes the use of an MMRN2 portion according to the first aspect of the invention, an agent according to the second aspect of the invention, a fusion protein according to the third aspect of the invention, an antibody according to the fifth aspect of the invention, a polynucleotide according to any of the fourth, sixth, eighth, tenth or twelfth aspects of the invention, a vector according to the thirteenth aspect of the invention, a cell according to the fourteenth aspect of the invention, a compound according to the seventh or ninth aspects of the invention, or a CAR according to the eleventh aspect of the invention, in the preparation of a medicament for combating a disease or condition in an individual, selected from the group consisting of cancer, psoriasis, menorrhagia, endometriosis, arthritis (both inflammatory and rheumatoid), macular degeneration, Paget's disease, retinopathy and its vascular complications (including proliferative and of prematurity, and diabetic retinopathy), benign vascular proliferations, fibroses, obesity and inflammation

Preferences for the MMRN2 portion, agent, fusion protein, antibody, polynucleotide, vector, cell, compound, and CAR in all medical use aspects of the invention include those described above in relation to the relevant aspect of the invention. The portion (e.g. a peptide of no more that 180 amino acids in length) may comprise the structure of Formula I. For example, the portion of MMRN2 may comprise or consist of the amino acid sequence of any of the MMRN2 portions listed in FIG. 8, or any part or variant of said portions. It is especially preferred if the portion of MMRN2 comprises or consists of the amino acid sequence of the human MMRN2 portion listed in FIG. 8, or a part of variant thereof. Similarly, the antibody may be an antibody that selectively binds such portions of MMRN2.

By “combating” we include the meaning that the method can be used to alleviate symptoms of the disorder (i.e. the method is used palliatively), or to treat the disorder, or to prevent the disorder (i.e. the method is used prophylactically).

Thus, the invention provides a method of treating an individual who has a disease in which angiogenesis contributes to pathology, the method comprising the step of administering to the individual an agent of the invention (e.g. a portion of MMRN2 of the first aspect of the invention or an agent according to the second aspect of the invention, or an antibody according to the fifth aspect of the invention or a CAR according to the eleventh aspect of the invention).

In any of the methods or uses of the invention described in this document herein, the individual is preferably a human. However, it will also be understood that the individual can be non-human, such as any non-human mammal, for example a horse, dog, pig, cow, sheep, rat, mouse, guinea pig or a primate.

Typically, in any of the methods or uses of the invention described in this document herein, the individual has a solid tumour, such as a tumour of the colon, rectum, ovary, liver, bladder, prostate, breast, kidney, pancreas, stomach, oesophagus, lung or thyroid.

Combination Therapy

According to a National Cancer Institute Press Release dated 14 Apr. 2005, updated 16 Jun. 2005, (“Bevacizumab Combined With Chemotherapy Improves Progression-Free Survival for Patients With Advanced Breast Cancer”), the angiogenesis inhibitor anti-VE.G.F monoclonal antibody bevacizumab improves the clinical outcome for a number of solid tumours when administered in combination with standard chemotherapy. Combinations that have been used include bevacizumab in combination with irinotecan, fluorouracil, and leucovorin; bevacizumab in combination with FOLFOX4 (a regimen of oxaliplatin, 5-fluorouracil and leucovorin); bevacizumab in combination with paclitaxel; and bevacizumab in combination with paclitaxel and carboplatin.

It is therefore appreciated that although the agents of the invention described above may be clinically effective in the absence of any other therapeutic agent (e.g. anti-cancer compound and/or anti-angiogenesis compound), it may be advantageous to administer these agents in conjunction with a further therapeutic agent (e.g. anticancer agent and/or anti-angiogenesis compound).

Accordingly, the invention includes a composition comprising an MMRN2 portion according to the first aspect of the invention, an agent according to the second aspect of the invention, a fusion protein according to the third aspect of the invention, an antibody according to the fifth aspect of the invention, a polynucleotide according to any of the fourth, sixth, eighth, tenth or twelfth aspects of the invention, a vector according to the thirteenth aspect of the invention, a cell according to the fourteenth aspect of the invention, a compound according to the seventh or ninth aspects of the invention, or a CAR according to the eleventh aspect of the invention, which composition further comprises at least one additional anti-cancer agent and/or at least one additional anti-angiogenic agent. It will be appreciated that the composition may be a pharmaceutical composition that also comprises a pharmaceutically acceptable diluent, carrier or excipient.

In an embodiment of the medical methods of the invention (e.g. method of inhibiting angiogenesis and method of combating a disease or condition involving unwanted, undesirable or inappropriate angiogenesis), the method may also comprise administering to the individual at least one further therapeutic agent (e.g. anticancer agent and/or ant-angiogenesis compound). The method may comprise administering to the individual a pharmaceutical composition containing the agent of the invention (e.g. MMRN2 portion or antibody or CAR), and the further therapeutic agent (e.g. anticancer agent and/or anti-angiogenesis compound). However, it is appreciated that the agent of the invention (e.g. MMRN2 portion or antibody or CAR) and further therapeutic agent (e.g. anticancer agent and/or anti-angiogenesis compound) may be administered separately, for instance by separate routes of administration. Thus, it is appreciated that the agent and the at least one further therapeutic agent can be administered sequentially or (substantially) simultaneously. They may be administered within the same pharmaceutical formulation or medicament or they may be formulated and administered separately.

In an embodiment of the medical uses of the invention, the medicament containing the agent of the invention may also comprise at least one further therapeutic agent (e.g. anticancer agent and/or anti-angiogenesis compound).

In another embodiment of the medical uses, the individual to be treated may be one who is administered at least one further therapeutic agent (e.g. anticancer agent and/or anti-angiogenesis compound). It is appreciated that the individual may be administered the further therapeutic agent (e.g. anticancer agent and/or anti-angiogenesis compound) at the same time as the medicament containing the agent of the invention, although the individual may have been (or will be) administered the further therapeutic agent (e.g. anticancer agent and/or anti-angiogenesis compound) before (or after) receiving the medicament containing the agent of the invention.

It will also be appreciated that the invention also provides a method of treatment, wherein a further therapeutic agent (e.g. anticancer agent and/or anti-angiogenesis compound) is administered to an individual in need thereof, wherein the individual is one who is administered an agent of the invention. The administration of the further therapeutic agent and agent of the invention may occur at the same time, although the individual may have been (or will be) administered the agent of the invention before (or after) receiving the medicament containing the further therapeutic agent (e.g. anticancer agent and/or anti-angiogenesis compound).

Preferably, the further therapeutic agent is an anti-cancer agent. The further anticancer agent may be selected from alkylating agents including nitrogen mustards such as mechlorethamine (HN₂), cyclophosphamide, ifosfamide, melphalan (L-sarcolysin) and chlorambucil; ethylenimines and methylmelamines such as hexamethylmelamine, thiotepa; alkyl sulphonates such as busulphan; nitrosoureas such as carmustine (BCNU), lomustine (CCNU), semustine (methyl-CCNU) and streptozocin (streptozotocin); and triazenes such as decarbazine (DTIC; dimethyltriazenoimidazole-carboxamide); antimetabolites including folic acid analogues such as methotrexate (amethopterin); pyrimidine analogues such as fluorouracil (5-fluorouracil; 5-FU), floxuridine (fluorodeoxyuridine; FUdR) and cytarabine (cytosine arabinoside); and purine analogues and related inhibitors such as mercaptopurine (6-mercaptopurine; 6-MP), thioguanine (6-thioguanine; TG) and pentostatin (2′-deoxycoformycin); natural products including vinca alkaloids such as vinblastine (VLB) and vincristine; epipodophyllotoxins such as etoposide and teniposide; antibiotics such as dactinomycin (actinomycin D), daunorubicin (daunomycin; rubidomycin), doxorubicin, bleomycin, plicamycin (mithramycin) and mitomycin (mitomycin C); enzymes such as L-asparaginase; and biological response modifiers such as interferon alphenomes; miscellaneous agents including platinum coordination complexes such as cisplatin (cis-DDP) and carboplatin; anthracenedione such as mitoxantrone and anthracycline; substituted urea such as hydroxyurea; methyl hydrazine derivative such as procarbazine (N-methylhydrazine, MIH); and adrenocortical suppressant such as mitotane (o,p′-DDD) and aminoglutethimide; taxol and analogues/derivatives; cell cycle inhibitors; proteosome inhibitors such as Bortezomib (Velcade®); signal transductase (e.g. tyrosine kinase) inhibitors such as Imatinib (Glivec®), COX-2 inhibitors, and hormone agonists/antagonists such as flutamide and tamoxifen. Particularly, tirapazamine may be utilised.

The clinically used anticancer agents are typically grouped by mechanism of action: Alkylating agents, Topoisomerase I inhibitors, Topoisomerase II inhibitors, RNA/DNA antimetabolites, DNA antimetabolites and Antimitotic agents. The US NIH/National Cancer Institute website lists 122 compounds (http://dtp.nci.nih.gov/docs/cancer/searches/standard_mechanism.html), all of which may be used in conjunction with an inhibitor of CLEC14A. They include Alkylating agents including Asaley, AZQ, BCNU, Busulfan, carboxyphthalatoplatinum, CBDCA, CCNU, CHIP, chlorambucil, chlorozotocin, cis-platinum, clomesone, cyanomorpholino-doxorubicin, cyclodisone, dianhydrogalactitol, fluorodopan, hepsulfam, hycanthone, melphalan, methyl CCNU, mitomycin C, mitozolamide, nitrogen mustard, PCNU, piperazine, piperazinedione, pipobroman, porfiromycin, spirohydantoin mustard, teroxirone, tetraplatin, picoplatin (SP-4-3) (cis-aminedichloro(2-methylpyridine)Pt(II)), thio-tepa, triethylenemelamine, uracil nitrogen mustard, Yoshi-864; anitmitotic agents including allocolchicine, Halichondrin B, colchicine, colchicine derivative, dolastatin 10, maytansine, rhizoxin, taxol, taxol derivative, thiocolchicine, trityl cysteine, vinblastine sulphate, vincristine sulphate; Topoisomerase I Inhibitors including camptothecin, camptothecin, Na salt, aminocamptothecin, 20 camptothecin derivatives, morpholinodoxorubicin; Topoisomerase II Inhibitors including doxorubicin, amonafide, m-AMSA, anthrapyrazole derivative, pyrazoloacridine, bisantrene HCL, daunorubicin, deoxydoxorubicin, mitoxantrone, menogaril, N,N-dibenzyl daunomycin, oxanthrazole, rubidazone, VM-26, VP-16; RNA/DNA antimetabolites including L-alanosine, 5-azacytidine, 5-fluorouracil, acivicin, 3 aminopterin derivatives, an antifol, Baker's soluble antifol, dichlorallyl lawsone, brequinar, ftorafur (pro-drug), 5,6-dihydro-5-azacytidine, methotrexate, methotrexate derivative, N-(phosphonoacetyI)-L-aspartate (PALA), pyrazofurin, trimetrexate; DNA antimetabolites including, 3-HP, 2′-deoxy-5-fluorouridine, 5-HP, alpha-TGDR, aphidicolin glycinate, ara-C, 5-aza-2′-deoxycytidine, beta-TGDR, cyclocytidine, guanazole, hydroxyurea, inosine glycodialdehyde, macbecin II, pyrazoloimidazole, thioguanine and thiopurine.

It is, however, preferred that the at least one further anticancer agent is selected from cisplatin; carboplatin; picoplatin; 5-flurouracil; paclitaxel; mitomycin C; doxorubicin; gemcitabine; tomudex; pemetrexed; methotrexate; irinotecan, fluorouracil and leucovorin; oxaliplatin, 5-fluorouracil and leucovorin; and paclitaxel and carboplatin.

When the further anticancer agent has been shown to be particularly effective for a specific tumour type, it may be preferred that the agent of the invention is used in combination with that further anticancer agent to treat that specific tumour type.

Preferred anti-angiogenesis compounds include bevacizumab (Avastin®); itraconazole; carboxyamidotriazole; TNP-470 (an analog of fumagillin); CM101; IFN-α; IL-12; platelet factor-4; suramin; SU5416; thrombospondin; VE.G.FR antagonists; angiostatic steroids+heparin; Cartilage-Derived Angiogenesis Inhibitory Factor; matrix metalloproteinase inhibitors; angiostatin; endostatin; 2-methoxyestradiol; tecogalan; tetrathiomolybdate; thalidomide; prolactin; αᵥβ₃ inhibitors; linomide; tasquinimod; ranibizumab; sorafenib; (Nexavar®); sunitinib (Sutent®); pazopanib (Votrient®); and everolimus (Afinitor®).

The invention also provides a kit of parts comprising: (i) an MMRN2 portion according to any of the first aspect of the invention; an agent according to the second aspect of the invention;

a fusion protein according to the third aspect of the invention; a nucleic acid according to the fourth, sixth, eighth, tenth and twelfth aspects of the invention; a vector according to the thirteenth aspect of the invention; a cell according to the fourteenth aspect of the invention; a compound according to the seventh or ninth aspects of the invention; a CAR according to the eleventh aspect of the invention; and (ii) at least one additional anti-cancer agent and/or at least one additional anti-angiogenic agent.

Formulations and Routes of Administration

An eighteenth aspect of the invention provides a pharmaceutical composition comprising an MMRN2 portion according to the first aspect of the invention, an agent according to the second aspect of the invention, a fusion protein according to the third aspect of the invention, an antibody according to the fifth aspect of the invention, a polynucleotide according to any of the fourth, sixth, eighth, tenth or twelfth aspects of the invention, a vector according to the thirteenth aspect of the invention, a cell according to the fourteenth aspect of the invention, a compound according to the seventh or ninth aspects of the invention, or a CAR according to the eleventh aspect of the invention, and a pharmaceutically acceptable diluent, carrier or excipient.

By “pharmaceutically acceptable” is included that the formulation is sterile and pyrogen free. Suitable pharmaceutical carriers, diluents and excipients are well known in the art of pharmacy. The carrier(s) must be “acceptable” in the sense of being compatible with the inhibitor and not deleterious to the recipients thereof. Typically, the carriers will be water or saline which will be sterile and pyrogen free; however, other acceptable carriers may be used.

In an embodiment, the pharmaceutical compositions or formulations of the invention are for parenteral administration, more particularly for intravenous administration. In a preferred embodiment, the pharmaceutical composition is suitable for intravenous administration to a patient, for example by injection.

Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents.

In an alternative preferred embodiment, the pharmaceutical composition is suitable for topical administration to a patient.

Preferably, the formulation is a unit dosage containing a daily dose or unit, daily sub-dose or an appropriate fraction thereof, of the active ingredient.

The agent or active ingredient may be administered orally or by any parenteral route, in the form of a pharmaceutical formulation comprising the active ingredient, optionally in the form of a non-toxic organic, or inorganic, acid, or base, addition salt, in a pharmaceutically acceptable dosage form. Depending upon the disorder and patient to be treated, as well as the route of administration, the compositions may be administered at varying doses.

In human therapy, the agent or active ingredient will generally be administered in admixture with a suitable pharmaceutical excipient, diluent or carrier selected with regard to the intended route of administration and standard pharmaceutical practice.

For example, the agent or active ingredient may be administered orally, buccally or sublingually in the form of tablets, capsules, ovules, elixirs, solutions or suspensions, which may contain flavouring or colouring agents, for immediate-, delayed- or controlled-release applications. The active ingredient may also be administered via intracavernosal injection.

Suitable tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycolate, croscarmellose sodium and certain complex silicates, and granulation binders such as polyvinylpyrrolidone, hydroxypropylmethylcellulose (HPMC), hydroxy-propylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, a cellulose, milk sugar or high molecular weight polyethylene glycols. For aqueous suspensions and/or elixirs, the compounds of the invention may be combined with various sweetening or flavouring agents, colouring matter or dyes, with emulsifying and/or suspending agents and with diluents such as water, ethanol, propylene glycol and glycerin, and combinations thereof.

The agent or active ingredient can also be administered parenterally, for example, intravenously, intra-arterially, intraperitoneally, intrathecally, intraventricularly, intrasternally, intracranially, intra-muscularly or subcutaneously, or they may be administered by infusion techniques. They are best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The formulations may be presented in unit-dose or multi-dose containers, for example sealed ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

For oral and parenteral administration to human patients, the daily dosage level of an agent, antibody or compound will usually be from 1 to 1,000 mg per adult (i.e. from about 0.015 to 15 mg/kg), administered in single or divided doses.

Thus, for example, the tablets or capsules of the agent or active ingredient may contain from 1 mg to 1,000 mg of agent or active agent for administration singly or two or more at a time, as appropriate. The physician in any event will determine the actual dosage which will be most suitable for any individual patient and it will vary with the age, weight and response of the particular patient. The above dosages are exemplary of the average case. There can, of course, be individual instances where higher or lower dosage ranges are merited and such are within the scope of this invention.

The agent or active ingredient can also be administered intranasally or by inhalation and are conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoro-ethane, a hydrofluoroalkane such as 1,1,1,2-tetrafluoroethane (HFA 134A3 or 1,1,1,2,3,3,3-heptafluoropropane (HFA 227EA3), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of an active ingredient and a suitable powder base such as lactose or starch. Such formulations may be particularly useful for treating solid tumours of the lung, such as, for example, small cell lung carcinoma, non-small cell lung carcinoma, pleuropulmonary blastoma or carcinoid tumour.

Aerosol or dry powder formulations are preferably arranged so that each metered dose or “puff' contains at least 1 mg of the inhibitor for delivery to the patient. It will be appreciated that the overall daily dose with an aerosol will vary from patient to patient, and may be administered in a single dose or, more usually, in divided doses throughout the day.

Alternatively, the agent or active ingredient can be administered in the form of a suppository or pessary, particularly for treating or targeting colon, rectal or prostate tumours.

The agent or active ingredient may also be administered by the ocular route. For ophthalmic use, the inhibitor can be formulated as, e.g., micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum. Such formulations may be particularly useful for treating solid tumours of the eye, such as retinoblastoma, medulloepithelioma, uveal melanoma, rhabdomyosarcoma, intraocular lymphoma, or orbital lymphoma.

The agent or active ingredient may be applied topically in the form of a lotion, solution, cream, ointment or dusting powder, or may be transdermally administered, for example, by the use of a skin patch. For application topically to the skin, the active ingredient can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water. Such formulations may be particularly useful for treating solid tumours of the skin, such as, for example, basal cell cancer, squamous cell cancer or melanoma.

For skin cancers, the agent or active ingredient can also be delivered by electroincorporation (EI). EI occurs when small particles of up to 30 microns in diameter on the surface of the skin experience electrical pulses identical or similar to those used in electroporation. In EI, these particles are driven through the stratum corneum and into deeper layers of the skin.

The particles can be loaded or coated with inhibitor or can simply act as “bullets” that generate pores in the skin through which the active ingredient can enter.

Formulations suitable for topical administration in the mouth include lozenges comprising the active ingredient in a flavoured basis, usually sucrose and acacia or tragacanth; pastilles comprising the agent or active ingredient in an inert basis such as gelatin and glycerin, or sucrose and acacia; and mouth-washes comprising the active ingredient in a suitable liquid carrier. Such formulations may be particularly useful for treating solid tumours of the mouth and throat.

In an embodiment, the agent or active ingredient may be delivered using an injectable sustained-release drug delivery system. These are designed specifically to reduce the frequency of injections. An example of such a system is Nutropin Depot which encapsulates recombinant human growth hormone (rhGH) in biodegradable microspheres that, once injected, release rhGH slowly over a sustained period.

The agent or active ingredient can be administered by a surgically implanted device that releases the drug directly to the required site, for example, into the eye to treat ocular tumours. Such direct application to the site of disease achieves effective therapy without significant systemic side-effects.

An alternative method for delivery of agents or active ingredients is the Regel injectable system that is thermo-sensitive. Below body temperature, Regel is an injectable liquid while at body temperature it immediately forms a gel reservoir that slowly erodes and dissolves into known, safe, biodegradable polymers. The active drug is delivered over time as the biopolymers dissolve.

Polypeptide pharmaceuticals can also be delivered orally. The process employs a natural process for oral uptake of vitamin B₁₂ in the body to co-deliver proteins and peptides. By riding the vitamin B₁₂ uptake system, the protein or peptide can move through the intestinal wall. Complexes are synthesised between vitamin B₁₂ analogues and the drug that retain both significant affinity for intrinsic factor (IF) in the vitamin B₁₂ portion of the complex and significant bioactivity of the drug portion of the complex.

Polynucleotides may be administered as a suitable genetic construct as described below and delivered to the patient where it is expressed. Typically, the polynucleotide in the genetic construct is operatively linked to a promoter which can express the compound in the cell. The genetic constructs of the invention can be prepared using methods well known in the art, for example in Sambrook et al (2001).

Although genetic constructs for delivery of polynucleotides can be DNA or RNA, it is preferred if they are DNA.

Preferably, the genetic construct is adapted for delivery to a human cell. Means and methods of introducing a genetic construct into a cell are known in the art, and include the use of immunoliposomes, liposomes, viral vectors (including vaccinia, modified vaccinia, lentivurus, parvovirus, retroviruses, adenovirus and adeno-associated viral (AAV) vectors), and by direct delivery of DNA, e.g. using a gene-gun and electroporation. Furthermore, methods of delivering polynucleotides to a target tissue of a patient for treatment are also well known in the art. In an alternative method, a high-efficiency nucleic acid delivery system that uses receptor-mediated endocytosis to carry DNA macromolecules into cells is employed. This is accomplished by conjugating the iron-transport protein transferrin to polycations that bind nucleic acids. High-efficiency receptor-mediated delivery of the DNA constructs or other genetic constructs of the invention using the endosome-disruption activity of defective or chemically inactivated adenovirus particles produced by the methods of Cotten et al (1992) Proc. Natl. Acad. Sci. USA 89, 6094-6098 may also be used. It will be appreciated that “naked DNA” and DNA complexed with cationic and neutral lipids may also be useful in introducing the DNA of the invention into cells of the individual to be treated. Non-viral approaches to gene therapy are described in Ledley (1995, Human Gene Therapy 6, 1129-1144).

Although for cancer/tumours of specific tissues it may be useful to use tissue-specific promoters in the vectors encoding a polynucleotide inhibitor, this is not essential, as the risk of expression of the active ingredient in the body at locations other than the cancer/tumour would be expected to be tolerable in compared to the therapeutic benefit to a patient suffering from a cancer/tumour. It may be desirable to be able to temporally regulate expression of the polynucleotide inhibitor in the cell, although this is also not essential.

The agents or active ingredients of the invention (i.e. portion of MMRN2, agent that inhibits the interaction between MMRN2 and CD93, antibody that selectively binds to a portion of MMRN2, fusion protein, CAR, compounds of invention and polynucleotides and vectors) may be lyophilised for storage and reconstituted in a suitable carrier prior to use. Any suitable lyophilisation method (e.g. spray drying, cake drying) and/or reconstitution techniques can be employed. It will be appreciated by those skilled in the art that lyophilisation and reconstitution can lead to varying degrees of protein activity loss and that use levels may have to be adjusted upward to compensate. In one embodiment, the lyophilised (freeze dried) active ingredient loses no more than about 20%, or no more than about 25%, or no more than about 30%, or no more than about 35%, or no more than about 40%, or no more than about 45%, or no more than about 50% of its activity (prior to lyophilisation) when re-hydrated.

Targeted Delivery of Cytotoxic Moieties

A nineteenth aspect of the invention provides a method of targeting a cytotoxic moiety to neovasculature in the body of an individual, the method comprising:

administering to the individual a compound comprising a compound according to the ninth aspect of the invention (i.e. (i) a MMRN2 portion according to the first or second aspect of the invention and (ii) a cytotoxic moiety).

This aspect of the invention includes a compound according to the ninth aspect of the invention for use in targeting a cytotoxic moiety to neovasculature in the body of an individual. This aspect of the invention further includes the use of a compound according to the ninth aspect of the invention in the preparation of a medicament for targeting a cytotoxic moiety to neovasculature in the body of an individual.

It is appreciated that targeting a cytotoxic agent to neovasculature will act to inhibit angiogenesis. Hence, this aspect of the invention includes a method of inhibiting angiogenesis in an individual, the method comprising: administering to the individual a compound according to the ninth aspect of the invention. Preferably, the neovasculature is tumour neovasculature and the angiogenesis is tumour angiogenesis.

This aspect of the invention also includes a compound according to the ninth aspect of the invention for use in inhibiting angiogenesis in an individual. This aspect of the invention also includes the use of a compound according to the ninth aspect of the invention in the preparation of a medicament for inhibiting angiogenesis in an individual.

Typically, the individual in the nineteenth aspect of the invention has a solid tumour, preferably such as those described above in relation to the fifteenth, sixteenth and seventeenth aspects of the invention.

It is appreciated that although targeting a cytotoxic moiety to tumour neovasculature to inhibit tumour neoangiogenesis may be clinically effective in the absence of any other anti-cancer compound, it may nevertheless be advantageous to administer the compounds in conjunction with a further anticancer agent. Accordingly, in an embodiment, the method may comprise administering to the individual a further anticancer agent.

Preferences for the further anticancer agent to be administered include any of the cytotoxic agents described above. For example, the anticancer agent may be any one or more of cisplatin; carboplatin; 5-flurouracil; paclitaxel; mitomycin C; doxorubicin; gemcitabine; tomudex; pemetrexed; methotrexate; irinotecan, fluorouracil and leucovorin; oxaliplatin, 5-fluorouracil and leucovorin; and paclitaxel and carboplatin.

The compound according to the ninth aspect of the invention and the further anticancer agent may be administered in the form of a pharmaceutical composition containing both of these components. However, it is appreciated that the compound and the further anticancer agent, may be administered separately, for instance by separate routes of administration. Thus it is appreciated that the compound and the at least one further anticancer agent can be administered sequentially or (substantially) simultaneously. They may be administered within the same pharmaceutical formulation or medicament or they may be formulated and administered separately.

Thus, the method may comprise administering the compound according to the ninth aspect of the invention to the individual wherein the individual is one who is administered a further anticancer agent. Similarly, the method may comprise administering a further anticancer to an individual wherein the individual is one who is administered the compound according to the ninth aspect of the invention.

The invention also provides a kit of parts comprising: (i) an MMRN2 portion according to any of the first or second aspects of the invention; a fusion protein according to the third aspect of the invention, a nucleic acid according to the fourth, sixth, eighth, tenth and twelfth aspects of the invention; a vector according to the thirteenth aspect of the invention; a cell according to the fourteenth aspect of the invention; a compound according to the seventh or ninth aspects of the invention; or a CAR according to the eleventh aspect of the invention, and (ii) a cytotoxic moiety.

Imaging, Detection and Diagnosis

A twentieth aspect of the invention provides a method of imaging neovasculature in the body of an individual the method comprising:

administering to the individual a compound according to the seventh aspect of the invention (i.e. (i) an MMRN2 portion according to the first or second aspects of the invention, and (ii) a detectable moiety), and imaging the detectable moiety in the body.

Typically, the individual has a solid tumour, preferably such as those described above, and the neovasculature of the tumour is imaged. Thus, the localisation of the portion of MMRN2 at a particular organ in the body indicates that the individual may have or may be developing a solid tumour at that organ. This method may be useful, for example, in determining the size of a previously diagnosed solid tumour, determining the effectiveness of a therapy against the solid tumour, or determining the extent of metastasis of the tumour. Methods for imaging the detectable moiety in the body are well known in the art, and include PET (positron emission tomography).

Accordingly, this aspect of the invention provides a method of detecting, diagnosing and prognosing a solid tumour in an individual, the method comprising: administering to the individual a compound according to the seventh aspect of the invention, and detecting the presence and/or location of the detectable moiety in the body.

In an embodiment, the method further comprises the step of detecting the location of the compound in the individual.

Preferences for the detectable moiety include those mentioned above, such as any of iodine-123, iodine-131, indium-111, fluorine-19, carbon-13, nitrogen-15, oxygen-17, technetium-99m, gadolinium, manganese or iron.

Preferably, the individual is a human.

Preferably, the individual has a solid tumour such as any of those described above.

The invention also provides a kit of parts comprising (i) an MMRN2 portion according to any of the first or second aspects of the invention; a fusion protein according to the third aspect of the invention, a nucleic acid according to the fourth, sixth, eighth, tenth and twelfth aspects of the invention; a vector according to the thirteenth aspect of the invention; a cell according to the fourteenth aspect of the invention; a compound according to the seventh or ninth aspects of the invention; or a CAR according to the eleventh aspect of the invention, and (ii) a detectable moiety.

Further Products

The inventors have characterised new molecular interactions between MMRN2, CLEC14A and CD93, and so it will be appreciated that complexes between the portion of MMRN2 of the invention and either of CLEC14A and CD93 will have utility in further elucidating the role of these molecules in angiogenesis.

Accordingly, a twenty-first aspect of the invention provides a complex comprising: (i) a MMRN2 portion according to the first or second aspect of the invention; and (ii) (a) CLEC14A or a portion or variant thereof, said portion or variant being capable of binding to MMRN2, and/or CD93 or a portion or variant thereof, said portion or variant being capable of binding to MMRN2.

Similarly, a twenty-second aspect of the invention provides a kit of parts comprising: (i) a MMRN2 portion according to the first or second aspect of the invention; and (ii) (a) CLEC14A or a portion or variant thereof, said portion or variant being capable of binding to MMRN2, and/or (b) CD93 or a portion or variant thereof, said portion or variant being capable of binding to MMRN2.

Likewise, a twenty-third aspect of the invention provides a nucleic acid molecule capable of expressing: (i) a MMRN2 portion according to the first or second aspect of the invention; and (ii) (a) CLEC14A or a portion or variant thereof, said portion or variant being capable of binding to MMRN2, and/or (b) CD93 or a portion or variant thereof, said portion or variant being capable of binding to MMRN2. It will be appreciated that a single nucleic acid molecule may be capable of expressing both (i) and (ii), or that two or three nucleic acid molecules may be capable of respectively expressing (i), (ii)(a) and (ii)(b).

Preferences for the MMRN2 portion according to the first or second aspects of the invention include those described above in relation to those aspects. For example, the portion of MMRN2 (e.g. a peptide of no more than 180 amino acids) may comprise the structure of Formula I. In a preferred embodiment, the portion of MMRN2 comprises or consists of the amino acid sequence of any of the MMRN2 portions listed in FIG. 8, or any part or variant of said portions. It is especially preferred if the portion of MMRN2 comprises or consists of the amino acid sequence of the human MMRN2 portion listed in FIG. 8, or a part of variant thereof. Preferences for the parts and variants include those described above.

By a portion or variant of CLEC14A or CD93 being capable of binding to MMRN2, we include any portion or variant of CLEC14A or CD93 that is capable of binding to MMRN2. Assessing protein-protein interactions is standard practice in the art any suitable method may be used.

Typically, the portion of CLEC14A or CD93 that is capable of binding to MMRN2 is at least 20 amino acid residues in length, and may be between 20 and 50 residues or between 50 and 100 residues or between 100 and 150 residues or between 150 and 200 residues in length, or more. In a particular embodiment, the portion of CLEC14A or CD93 that is capable of binding to MMRN2 is less than 400, 350, 300, 250, 150, 140, 130, 110, 100, 95, 90 or 85 amino acid residues in length.

It is preferred that the portion of CLEC14A that is capable of binding to MMRN2 is a portion of, or that the portion contains, the extracellular region of CLEC14A (residues 22-396), or that the portion is a portion of, or that the portion contains the C-type lectin like domain (residues 32-173), or that the portion is a portion of, or that the portion contains the long-loop region of CLEC14A (residues E97-C143) or that the portion is a portion of, or that the portion contains, residues 97-108 of the C-type lectin like domain.

It is preferred that the portion of CD93 that is capable of binding to MMRN2 is a portion of, or that the portion contains, the extracellular region of CD93 (residuesA22-K580), or that the portion is a portion of, or that the portion contains the C-type lectin like domain (residues A22-N174), or that the portion is a portion of, or that the portion contains the long-loop region of CLEC14A (residues Q98-C142) or that the portion is a portion of, or that the portion contains, residues 97-108 of the C-type lectin like domain.

By a variant of CLEC14A or CD93 that is capable of binding to MMRN2, we include variants of CLEC14A that have at least 60% sequence identity to human CLEC14A or human CD93, the sequences of which are provided in FIGS. 16 and 17, for example variants with at least 65%, 70%, 75%, 80%, 85%, 90%, 95%, 96%, 97%, 98% or 99% sequence identity to human CLEC14A or human CD93. It is preferred if the variant polypeptide has a consecutive region of at least 20 amino acid residues, more preferably at least 50 residues, of the sequence of the CLEC14A polypeptide listed in FIG. 16 or the CD93 polypeptide listed in FIG. 17. Such variants may be made, for example, using the methods of recombinant DNA technology, protein engineering and site-directed mutagenesis which are well known in the art.

It will be appreciated that the portions of CLEC14A or CD93 described above may also be portions of CLEC14A or CD93 variants. Generally, the portions of CLEC14A or CD93 have at least 60% sequence identity to human CLEC14A or CD93 respectively, the sequences of which are provided in FIGS. 16 and 17, for example at least 65%, 70%, 75%, 80%, 85%, 90%, 95%, 96%, 97%, 98%, or 99% sequence identity over the length of the portion.

Conveniently, the complex of the twenty-first aspect of the invention is produced by expressing the portion of MMRN2, and either CLEC14A and/or CD93 (or portions or variants thereof) separately, and adding the two proteins together after expression under conditions appropriate for complex formation. Alternatively, a cell may be engineered to overexpress the portion of MMRN2 and either CLEC14A and/or CD93 (or portions or variants thereof) using standard molecular biology techniques, such that the complex can be recovered from the cell lysate. Preferably, the complex is soluble. Typically, the proteins are manufactured in E. coli and purified by tagging them with histidine tags and using nickel beads to isolate the recombinant proteins. Similarly, differently epitope tagged versions of the proteins can be expressed in and purified from cells.

It is appreciated that such complexes may be useful in the screening methods of the invention described below. Conveniently, either one or both parts of the complex are detectably labelled so that the presence of the complex in a sample or cell can readily be detected. Examples of labels include peptide labels, chemical labels, fluorescent labels or radio labels.

As described in Example 1, the inventors have conducted various mutagenesis studies to investigate the interaction between MMRN2 and each of CLEC14A and CD93. Specifically, they have identified particular regions in all proteins that are important to their interactions.

Accordingly, a twenty-fourth aspect of the invention provides a mutant MMRN2 polypeptide which has reduced binding to CLEC14A relative to wild type MMRN2.

By “reduced binding to CLEC14A” we include the meaning that the mutant MMRN2 binds to CLEC14A with less affinity than wild type MMRN2 binds to CLEC14A. Typically, the mutant MMRN2 binds to CLEC14A with at least 2, or at least 5, or at least 10, or at least 50 times less affinity than does the wild type MMRN2 binds to CLEC14A. Most preferably, binding between CLEC14A and the mutant MMRN2 is reduced to an undetectable level.

In an embodiment, the mutant MMRN2, when compared to the corresponding wild type MMRN2, comprises one or more different amino acids in the region of MMRN2 corresponding to the region spanning amino acid residues 588-620 of human MMRN2, for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, or 31 different amino acids. Also included are mutant MMRN2 polypeptides wherein the region corresponding to the region spanning amino acid residues 588-620 of human MMRN2, or part thereof, is deleted.

In another embodiment, the mutant MMRN2 is a portion of MMRN2 consisting of the region of MMRN2 corresponding to the region spanning amino acid residues 495-603 or 487-603 or 604-674 of human MMRN2.

In yet another embodiment, the mutant MMRN2 is one which, when compared to the corresponding wild type MMRN2, has a different amino acid at a position which corresponds to any one or more of (e.g. at least 2, 3, 4, 5, 6, 7, 8, 9, 10, 11, 12, 13, 14, 15, 16, or 17 of, or all 18 of) the following positions according to the numbering of the human MMNR2 as set out in FIG. 15: Leu-536, Val-575, Leu-578, Val-589, Leu-592, Phe-596, Leu-599, Leu-600, Asp-602, Ala-603, Arg-605, His-606, Leu-610, Leu-613, Gly-615, Glu-616, Glu-617, and Leu-658. Additionally or alternatively, the mutant MMRN2 is one which, when compared to the corresponding wild type MMRN2, has a different amino acid at a position which corresponds to any one or more of (e.g. at least 2, 3, 4, 5, 6, 7, 8, 9, 10, or 11 of, or all 12 of) the following positions according to the numbering of the human MMNR2 as set out in FIG. 15: Leu-497, Glu-506, Gln-527, Val-540, Ala-546, Val-609, Glu-620, Gln-636, Ile-637, Leu-641, Leu-648 and Glu-666.

It will be appreciated that the mutant MMRN2 is a mutant of a particular species of MMRN2 (e.g. human), and so, apart from the mutation that is causative for the reduction in binding to CLEC14A, the mutant MMRN2 and wild type MMRN2 will share the same characteristics (e.g. structural and functional characteristics) of MMRN2 of whichever species.

A twenty-fifth aspect of the invention provides a mutant CLEC14A polypeptide which has reduced binding to MMRN2 relative to wild type CLEC14A, wherein the cysteine corresponding to cysteine-103 of human CLEC14A is mutated and/or the cysteine corresponding to cysteine-138 of human CLEC14A is mutated.

Preferably, the one or both cysteine residues are mutated to serine, although it is appreciated that any other amino acid may be used provided that binding to MMRN2 is still reduced compared to the wild type CLEC14A.

By “reduced binding to MMRN2” we include the meaning that the mutant CLEC14A binds to MMRN2 with less affinity than wild type CLEC14A binds to MMRN2. Typically, the mutant CLEC14A binds to MMRN2 with at least 2, or at least 5, or at least 10, or at least 50 times less affinity than does the wild type CLEC14A binds to MMRN2. Most preferably, binding between MMRN2 and the mutant CLEC14A is reduced to an undetectable level.

It will be appreciated that the mutant CLEC14A is a mutant of a particular species of CLEC14A (e.g. human), and so, apart from the mutation that is causative for the reduction in binding to MMRN2, the mutant CLEC14A and wild type CLEC14A will share the same characteristics (e.g. structural and functional characteristics) of CLEC14A of whichever species.

A twenty-sixth aspect of the invention provides a mutant CD93 polypeptide which has reduced binding to MMRN2 relative to wild type CD93

By “reduced binding to MMRN2” we include the meaning that the mutant CD93 binds to MMRN2 with less affinity than wild type CD93 binds to MMRN2. Typically, the mutant CD93 binds to MMRN2 with at least 2, or at least 5, or at least 10, or at least 50 times less affinity than does the wild type CD93 binds to MMRN2. Most preferably, binding between MMRN2 and the mutant CD93 is reduced to an undetectable level.

In an embodiment, the mutant CD93 is one which, when compared to the corresponding wild type CD93, comprises one or more different amino acids in the region of CD93 corresponding to the region spanning amino acid residues 97-108 of human CD93. Thus, the mutant may have at least 2, 3, 4, 5, 6, 7, 8, 9, 10 or 11 different amino acids in this region, or all 12 amino acids may be replaced. Also included are mutant CD93 polypeptides wherein the region corresponding to the region spanning amino acid residues 97-108 of human CD93, or part thereof, is deleted.

In an additional or alternative embodiment, the mutant CD93 is one which, when compared to the corresponding wild type CD93, the cysteine corresponding to cysteine-104 of human CD93 is mutated and/or the cysteine corresponding to cysteine-136 of human CD93 is mutated.

By “mutation” we include insertions, deletions and substitutions, either conservative or non-conservative. By “conservative substitutions” is intended combinations such as Gly, Ala; Val, Ile, Leu; Asp, Glu; Asn, Gin; Ser, Thr; Lys, Arg; and Phe, Tyr.

The mutants of the twenty-fourth to twenty-sixth aspects of the invention may be produced in any suitable way and provided in any suitable form. For example, methods of protein-engineering or conventional site-directed mutagenesis may be employed, or polymerase chain reaction-based procedures well known in the art may be used. Typically, the proteins are manufactured in E. coli and purified by tagging them with histidine tags and using nickel beads to isolate the recombinant proteins. Conveniently, the proteins are detectably labelled. Examples of such labels are well known in the art and include peptide labels, chemical labels, fluorescent labels, enzyme labels or radio labels.

For the avoidance of doubt, the invention does not include naturally occurring variants of the wild type MMRN2 polypeptide, the wild type CLEC14A polypeptide or the wild type CD93 polypeptide. The mutants are typically recombinantly produced.

The invention includes a nucleic acid molecule encoding any of the mutant polypeptides of the twenty-fourth to twenty-sixth aspects of the invention. The nucleic acid molecule may be incorporated into a vector such as an expression vector. Further details of what the nucleic acid and vector may be include those described above. It will also be appreciated that the invention provides a cell comprising the nucleic acid molecule or vector.

Screening Methods

A twenty-seventh aspect of the invention provides a method of identifying a portion of MMRN2 or a variant thereof, which portion may be useful in modulating angiogenesis or in combating cancer, or a lead compound for the identification of an agent that may be useful in modulating angiogenesis or in combating cancer, the method comprising:

providing CLEC14A or a portion or variant thereof, said portion or variant being capable of binding to MMRN2;

providing a candidate portion of MMRN2 or a variant thereof; and

determining whether the candidate portion modulates binding of CLEC14A or the portion or variant thereof, to MMRN2, or a portion or variant thereof, said portion or variant being capable of binding to CLEC14A.

By “modulating angiogenesis”, we include the meaning of inhibiting or enhancing angiogenesis.

By CLEC14A polypeptide, we include human CLEC14A having the sequence listed in FIG. 16, or an orthologue or naturally occurring variant thereof. Preferences for the portion or variant of CLEC14A include those described above in relation to the twenty-first aspect of the invention. It is particularly preferred if a portion of CLEC14A is used in the method of the twenty-seventh aspect of the invention, and still more preferred if the portion comprises or consists of the long-loop region of CLEC14A (residues E97-C143) or the region io corresponding to the region spanning residues 97-108 of the C-type lectin like domain of human CLEC14A.

By MMRN2 polypeptide, we include human MMRN2 having the sequence listed in FIG. 15, or an orthologue or naturally occurring variant thereof.

By portion or variant of MMRN2 being capable of binding to CLEC14A, we include any portion or variant of MMRN2 that is capable of binding to CLEC14A.

Typically, the portion of MMRN2 that is capable of binding to CLEC14A is at least 20 amino acid residues in length, and may be between 20 and 50 residues or between 50 and 100 residues or between 100 and 150 residues or between 150 and 200 residues in length, or more. In a particular embodiment, the portion of MMRN2 that is capable of binding to CLEC14A is less than 800, 700, 600, 500, 400, 350, 300, 250, 150, 140, 130, 110, 100, 95, 90 or 85 amino acid residues in length.

By a variant of MMRN2 that is capable of binding to CLEC14A, we include variants of MMRN2 that have at least 60% sequence identity to human MMRN2, the sequence of which is provided in FIG. 15, for example variants with at least 65%, 70%, 75%, 80%, 85%, 90%, 95%, 96%, 97%, 98% or 99% sequence identity to human MMRN2. It is preferred if the variant polypeptide has a consecutive region of at least 20 amino acid residues, more preferably at least 50 residues, of the sequence of the MMRN2 polypeptide listed in FIG. 15. Such variants may be made, for example, using the methods of recombinant DNA technology, protein engineering and site-directed mutagenesis which are well known in the art.

It will be appreciated that the portions of MMRN2 described above may also be portions of MMRN2 variants. Generally, the portions of MMRN2 have at least 60% sequence identity to human MMRN2, the sequence of which is provided in FIG. 15, for example at least 65%, 70%, 75%, 80%, 85%, 90%, 95%, 96%, 97%, 98%, or 99% sequence identity over the length of the portion.

In a preferred embodiment, the portion of MMRN2 used in the method of the twenty-seventh aspect of the invention (i.e. not the candidate portion of MMRN2), is the portion of MMRN2 of the first or second aspects of the invention, and the preferences mentioned above in relation to those aspects equally apply here.

The candidate portion of MMRN2 may be any portion of wild type MMRN2 or any portion of a variant of wild type MMRN2.

In an embodiment, the method of the twenty-seventh aspect of the invention further comprises:
determining whether the candidate portion of MMRN2 or variant thereof,
modulates binding of CD93, or a portion or variant thereof, said portion or variant being capable of binding to MMRN2,
to MMRN2 or a portion or variant thereof, said portion or variant being capable of binding to CD93.

By CD93 polypeptide, we include human CD93 having the sequence listed in FIG. 17, or an orthologue or naturally occurring variant thereof. Preferences for the portion or variant of CD93 include those described above in relation to the twenty-first aspect of the invention.

A twenty-eighth aspect of the invention provides a method of identifying an agent that may be useful in modulating angiogenesis or in combating cancer, or a lead compound for the identification of an agent that may be useful in modulating angiogenesis or in combating cancer, the method comprising:
providing CD93 or a portion or variant thereof, said portion or variant being capable of binding to MMRN2;
providing a candidate agent; and
determining whether the candidate agent modulates binding of CD93 or the portion or variant thereof, to MMRN2, or a portion or variant thereof, said portion or variant being capable of binding to CD93.

In an embodiment, the method further comprises:
determining whether the candidate agent
modulates binding of CLEC14A, or a portion or variant thereof, said portion or variant being capable of binding to MMRN2,
to MMRN2 or a portion or variant thereof, said portion or variant being capable of binding to CLEC14A.

Preferences for CD93 or portion or variant thereof, and MMRN2 or portion or variant thereof include those described above in relation to the twenty-seventh aspect of the invention.

The candidate agent may be any of an antibody, a peptide, a peptidomimetic, a natural product, a carbohydrate, an aptamer or a small organic molecule.

In an embodiment, the candidate agent is an antibody that selectively binds the CD93 polypeptide, or a fragment thereof, or an antibody that selectively binds the MMRN2 polypeptide, or a fragment thereof, or a portion of either CD93 or MMRN2.

In another embodiment, the candidate agent may be a peptide. Suitable peptides may be identified by methods such as phage display of peptide libraries (Scott & Smith (1990) “Searching for peptide ligands with an epitope library.” Science 249: 386-390; Felici et al (1995) “Peptide and protein display on the surface of filamentous bacteriophage.” Biotechnol. Annu. Rev. 1: 149-183); and Collins et a/ (2001) “Cosmix-plexing: a novel recombinatorial approach for evolutionary selection from combinatorial libraries.” J. Biotechnol. 74: 317-338); including in vivo panning (Pasqualini et a/ (1997) “αav inte.g.rins as receptors for tumor targeting by circulating ligands. Nature Biotechnol. 15: 542-546), and solid-phase parallel synthesis (Frank (2002) “The SPOT-synthesis technique. Synthetic peptide arrays on membrane supports—principles and applications.” J. Immunol. Methods 267: 13-26; and Pinilla et al (2003) “Advances in the use of synthetic combinatorial chemistry: mixture-based libraries.” Nature Med. 9: 118-122). The dissociation constants of peptides are typically in the micromolar range, although avidity can be improved by multimerization (Terskikh et al (1997) “Peptabody”: a new type of high avidity binding protein. Proc. Natl Acad. Sci. USA 94, 1663-1668; and Wrighton et al (1997) “Increased potency of an erythropoietin peptide mimetic through covalent dimerization. Nature Biotechnol. 15, 1261-1265).

The primary ligands of C-type lectins are carbohydrates, even though binding of other proteins, lipids or inorganic compounds has been shown. Thus, in another embodiment, the candidate agent may be a carbohydrate, or a molecule containing carbohydrate moieties such as a glycoprotein or gycolipid. It is appreciated that carbohydrate recognition and binding by C-type lectins is calcium dependant. Thus, in this embodiment, the method is carried out in the presence of calcium ions.

In still another embodiment, the candidate agent may be an aptamer, i.e. a single-stranded DNA molecule that folds into a specific ligand-binding structure. Suitable aptamers that bind to the CD93 polypeptide, the CLEC14A polypeptide or to the MMRN2 polypeptide, or a fragment thereof, may be identified by methods such as in vitro selection and amplification (Ellington & Szostak (1992) “Selection in vitro of single stranded DNA molecules that fold into specific ligand binding structures.” Nature 355: 850-852; and Daniels et al (2003) “A tenascin-C aptamer identified by tumor cell SELEX: systematic evolution of ligands by exponential enrichment.” Proc. Natl Acad. Sci. USA 100, 15416-15421). The aptamer may be a nuclease-stable ‘Spiegelmer (Helmling et al (2004) “Inhibition of ghrelin action in vitro and in vivo by an RNA-Spiegelmer.” Proc. Natl Acad. Sci. USA 101: 13174-13179). Aptamers typically have dissociation constants in the micromolar to the subnanomolar range.

In yet another embodiment, the candidate agent may be a small organic molecule. Suitable small molecules may be identified by methods such as screening large libraries of compounds (Beck-Sickinger & Weber (2001) Combinational Strategies in Biology and Chemistry (John Wiley & Sons, Chichester, Sussex); by structure-activity relationship by nuclear magnetic resonance (Shuker et al (1996) “Discovering high-affinity ligands for proteins: SAR by NMR. Science 274: 1531-1534); encoded self-assembling chemical libraries Melkko et al (2004) “Encoded self-assembling chemical libraries.” Nature Biotechnol. 22: 568-574); DNA-templated chemistry (Gartner et al (2004) “DNA-templated organic synthesis and selection of a library of macrocycles. Science 305: 1601-1605); dynamic combinatorial chemistry (Ramstrom & Lehn (2002) “Drug discovery by dynamic combinatorial libraries.” Nature Rev. Drug Discov. 1: 26-36); tethering (Arkin & Wells (2004) “Small-molecule inhibitors of protein-protein interactions: progressing towards the dream. Nature Rev. Drug Discov. 3: 301-317); and speed screen (Muckenschnabel et al (2004) “SpeedScreen: label-free liquid chromatography-mass spectrometry-based high-throughput screening for the discovery of orphan protein ligands.” Anal. Biochem. 324: 241-249). Typically, small organic molecules will have a dissociation constant for the polypeptide in the nanomolar range, particularly for antigens with cavities. The benefits of most small organic molecule binders include their ease of manufacture, lack of immunogenicity, tissue distribution properties, chemical modification strategies and oral bioavailability. Small molecules with molecular weights of less than 5000 daltons are preferred, for example less than 400,3000,2000, or 1000 daltons, or less than 500 daltons.

The capability of a candidate portion of MMRN2 or candidate agent to modulate binding of the particular interactions may be assessed by any method of detecting/measuring a protein/protein interaction or other compound/protein interaction, as discussed further below. Suitable methods include methods such as, for example, yeast two-hybrid interactions, co-purification, ELISA, co-immunoprecipitation and surface plasmon resonance methods. Thus, the candidate agent may be considered capable of modulating binding between the particular proteins if the interaction between those proteins as determined by ELISA, co-immunoprecipitation or surface plasmon resonance methods or by a yeast two-hybrid interaction or a copurification method, is changed (e.g. increased or decreased) compared to the interaction between those proteins in the absence of the candidate portion of MMRN2 or the candidate agent. It is preferred that the interaction can be detected using a surface plasmon resonance method. Surface plasmon resonance methods are well known to those skilled in the art. Techniques are described in, for example, O'Shannessy DJ (1994) “Determination of kinetic rate and equilibrium binding constants for macromolecular interactions: a critique of the surface plasmon resonance literature” Curr Opin Biotechnol. 5(1):65-71; Fivash et al (1998) “BlAcore for macromolecular interaction.” Curr Opin Biotechnol. 9(1):97-101; Malmqvist (1999) “BIACORE: an affinity biosensor system for characterization of biomolecular interactions.” Biochem Soc Trans. 27(2):335-40.

It is appreciated that screening assays which are capable of high throughput operation are particularly preferred. Examples may include cell based assays and protein-protein binding assays. An SPA-based (Scintillation Proximity Assay; Amersham International) system may be used.

Other methods of detecting polypeptide/polypeptide interactions include ultrafiltration with ion spray mass spectroscopy/HPLC methods or other physical and analytical methods. Fluorescence Energy Resonance Transfer (FRET) methods, for example, well known to those skilled in the art, may be used, in which binding of two fluorescent labelled entities may be measured by measuring the interaction of the fluorescent labels when in close proximity to each other.

It will be appreciated that the candidate portion of MMRN2 or the candidate agent may be added to one or the other of the members of the protein pair whose interaction is monitored (e.g. CLEC14A and MMRN2) before addition to the other member, and vice versa, and its effect on binding assessed.

Conveniently, at least one or other of members of the protein pair whose interaction is monitored (e.g. CLEC14A and MMRN2 (or portions or variants thereof)) are detectably labelled so as to facilitate detection of their binding and consequently the effect of the candidate agent. Examples of suitable labels include a peptide label, a nucleic acid label

(Kerr et al (1993) JACS vol. 115, p. 2529-2531; and Brenner & Lerner (1992) Proc. Natl. Acad. Sci. USA vol. 89, p. 5381-5383), a chemical label (Ohlmeyer et al (1993) Proc. Natl. Acad. Sci. USA vol. 90, p. 109222-10926; and Maclean et al (1997) Proc. Natl. Acad. Sci. USA vol. 94, p. 2805-2810); a fluorescent label (Yamashita & Weinstock (SmithKline Beecham Corporation), WO95/32425 (1995); and Sebestyen et al (1993) Pept. Proc. Eur. Pept. Symp. 22nd 1992, p. 63-64), or a radio frequency tag (Nicolaou et al (1995) Angew. Chem. Int. Ed. Engl. vol. 34, p. 2289-2291; and Moran et a/ (1995) JACS vol. 117, p. 10787-10788).

In one embodiment, the candidate portion of MMRN2 or candidate agent is one that reduces the level of binding between CLEC14A, or the portion or variant thereof, to MMRN2, or the portion or variant thereof. Additionally or alternatively, the candidate portion of MMRN2 or candidate agent is one that reduces the level of binding between CD93, or the portion or variant thereof, to MMRN2, or the portion or variant thereof. In both cases, the portion or agent may be useful in combating any disease or condition involving unwanted, undesirable or inappropriate angiogenesis, or may be a lead compound to the identification of an agent that is so useful

Preferably, the candidate portion or candidate agent reduces the level of binding between CLEC14A and MMRN2 (or portion(s) or variant(s) thereof) and/or the level of binding between CD93 and MMRN (or portion(s) or variant(s) thereof) by at least 10%, 20%, 30%, 40% or 50%, and more preferably the candidate agent is one that reduces the level of binding between CLEC14A and MMRN2 (or portion(s) or variant(s) thereof) and/or the level of binding between CD93 and MMRN (or portion(s) or variant(s) thereof) by at least 70%, 80%, 90%, 95% or 99%, compared to the level of binding in the absence of the candidate agent. Most preferably, the agent is one that reduces the level of binding between CLEC14A and MMRN2 (or portion(s) or variant(s) thereof) and/or the level of binding between CD93 and MMRN (or portion(s) or variant(s) thereof) to an undetectable level, or eliminates binding between CLEC14A and MMRN2 (or portion(s) or variant(s) thereof) and/or the level of binding between CD93 and MMRN (or portion(s) or variant(s) thereof).

It is appreciated that the identification of a candidate portion of MMRN2 or candidate agent that modulates binding of CLEC14A, or the portion or variant thereof, to MMRN2, or the portion or variant thereof and/or modulates binding of CD93, or the portion or variant thereof, to MMRN2, or the portion or variant thereof, may be an initial step in a drug screening pathway, and the identified agents may be further selected e.g. for the ability to inhibit angiogenesis and/or for the ability to inhibit tumour growth. Thus, the method may further comprise the step of testing the candidate portion or agent in an angiogenesis assay and/or testing the candidate agent for efficacy in an animal model of a solid tumour.

Methods and assays for determining the rate or level of angiogenesis, and hence for determining whether and to what extent a candidate agent inhibits angiogenesis, are known in the art. For example, U.S. Pat. No 6,225,118, incorporated herein by reference, describes a multicellular ex vivo assay for modelling the combined stages of angiogenesis namely the proliferation, migration and differentiation stages of cell development. The AngioKit, Catalogue No. ZHA-1000, by TCS CellWorks Ltd, Buckingham MK18 2LR, UK, is a suitable model of human angiogenesis for analysing the anti-angiogenic properties of compounds. The rate or level of angiogenesis can also be determined using the aortic ring assay and the sponge angiogenesis assay that are well known in the art.

Assays for endothelial cell proliferation, migration and invasion are also useful as angiogenesis assays. Suitable assays for endothelial cell proliferation and migration are known to a person of skill in the art. Suitable assays for endothelial cell invasion are also known to a person of skill in the art and include the BD BioCoat™ Angiogenesis System for Endothelial Cell Invasion which is available as Catalogue Nos. 354141 and 354142 from BD Biosciences, Bedford, Mass., USA, and the QCM™ Endothelial Cell Invasion Assay (EMD Millipore).

It is appreciated that these methods may be a drug screening methods, a term well known to those skilled in the art, and the candidate agent may be a drug-like compound or lead compound for the development of a drug-like compound.

The term “drug-like compound” is well known to those skilled in the art, and may include the meaning of a compound that has characteristics that may make it suitable for use in medicine, for example as the active ingredient in a medicament. Thus, for example, a drug-like compound may be a molecule that may be synthesised by the techniques of organic chemistry, less preferably by techniques of molecular biology or biochemistry, and is preferably a small molecule, which may be of less than 5000 Daltons and which may be water-soluble. A drug-like compound may additionally exhibit features of selective interaction with a particular protein or proteins and be bioavailable and/or able to penetrate target cellular membranes or the blood:brain barrier, but it will be appreciated that these features are not essential.

The term “lead compound” is similarly well known to those skilled in the art, and may include the meaning that the compound, whilst not itself suitable for use as a drug (for example because it is only weakly potent against its intended target, non-selective in its action, unstable, poorly soluble, difficult to synthesise or has poor bioavailability) may provide a starting-point for the design of other compounds that may have more desirable characteristics.

In an embodiment, the identified agent is modified, and the modified agent is tested for the ability to modulate binding between CLEC14A and MMRN2 (or portion(s) or variant(s) thereof) and/or CD93 and MMRN2 (or portion(s) or variant(s) thereof).

It is appreciated that the screening methods can be used to identify agents that may be useful in combating any disease or condition involving unwanted, undesirable or inappropriate angiogenesis, such as solid tumours. Thus, the screening methods preferably also comprise the further step of testing the identified agent or the modified agent for efficacy in an animal model of cancer, particularly a solid tumour. Suitable models are known in the art and include Lewis lung carcinoma subcutaneous implants in mice (homograft in Black 57 mice) or HT29 xenografts subcutaneous implants in nude mice.

The methods may comprise the further step of synthesising and/or purifying the identified agent or the modified agent. The methods may further comprise the step of formulating the agent into a pharmaceutically acceptable composition.

Agents may also be subjected to other tests, for example toxicology or metabolism tests, as is well known to those skilled in the art.

The invention includes a method for preparing an anti-angiogenic compound that may be useful in the treatment of any disease or condition involving unwanted, undesirable or inappropriate angiogenesis, the method comprising identifying an agent using the screening methods described above and synthesising, purifying and/or formulating the identified agent.

The invention includes a method for preparing an anti-cancer compound that may be useful in the treatment of a solid tumour, the method comprising identifying an agent using the screening methods described above and synthesising, purifying and/or formulating the identified agent.

The invention also includes a method of making a pharmaceutical composition comprising the step of mixing the agent identified using the methods described above with a pharmaceutically acceptable carrier.

All of the documents referred to herein are incorporated herein, in their entirety, by reference.

The invention will now be described by reference to the following Figures and Examples.

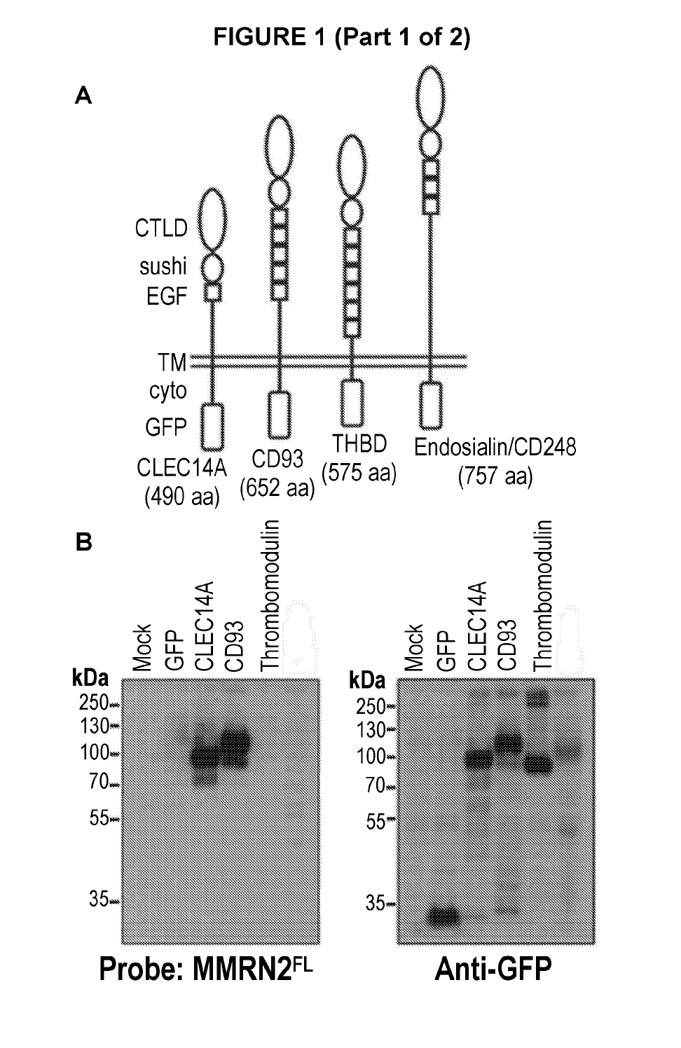

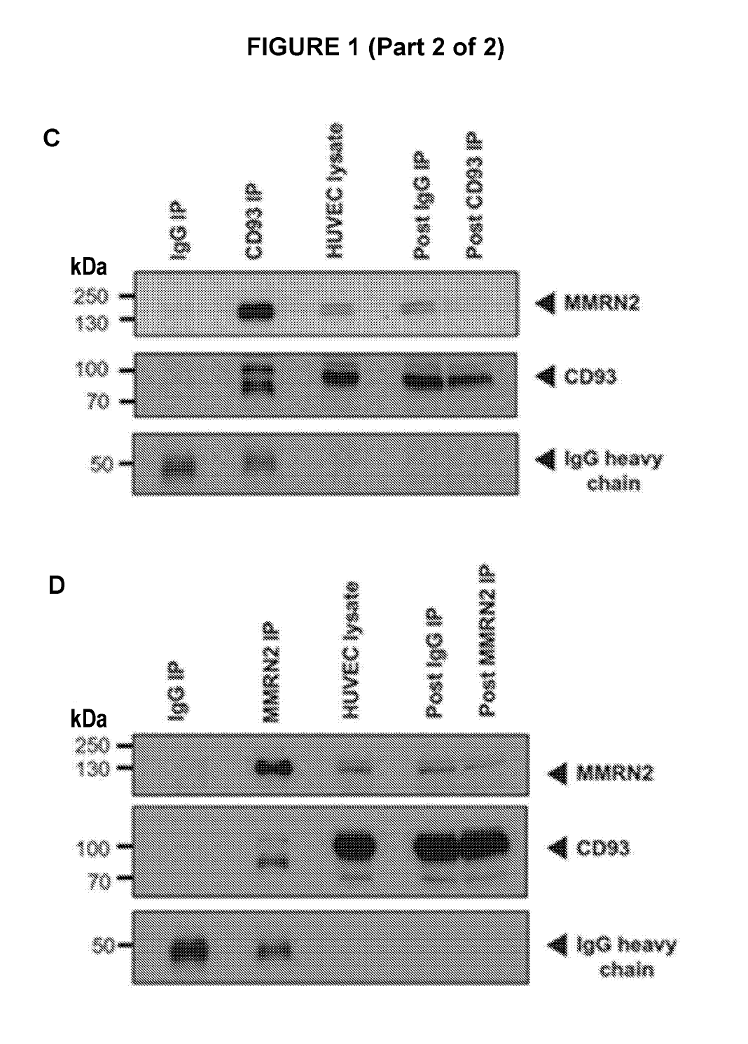

FIG. 1. CD93 directly binds to MMRN2. (A) Diagrams of GFP tagged CTLD group 14 family members showing domain structure and relative size with number of amino acids (aa) without the GFP tag. C-type lectin domain (CTLD), sushi, epidermal growth factor (E.G.F) domain, transmembrane (TM), cytoplasmic tail (cyto) and green fluorescent protein (GFP) tag are displayed. (B) HEK293T cells were transfected with GFP tagged group 14 family members, lysates were separated by SDS-PAGE under non-reduced conditions and far western blotted with MMRN2^{FL}, showing MMRN2^{FL} binds CLEC14A and CD93, GFP blots confirmed expression of all proteins. (C) Immunoprecipitations of CD93 using monoclonal R139 antibody co-immunoprecipitates MMRN2 from HUVEC lysates. (D) Immunoprecipitations of MMRN2 using mouse polyclonal antibodies co-immunoprecipitates CD93 from HUVEC lysates. IgG heavy chains included as loading control. CD93 was detected using goat polyclonal antibodies in each immunoprecipitation experiment.

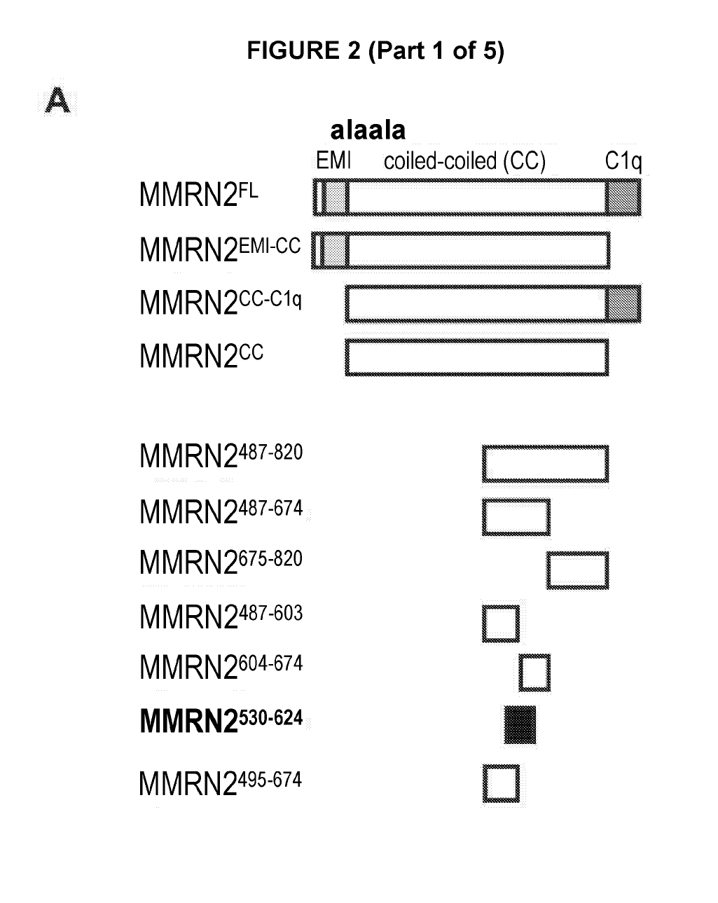

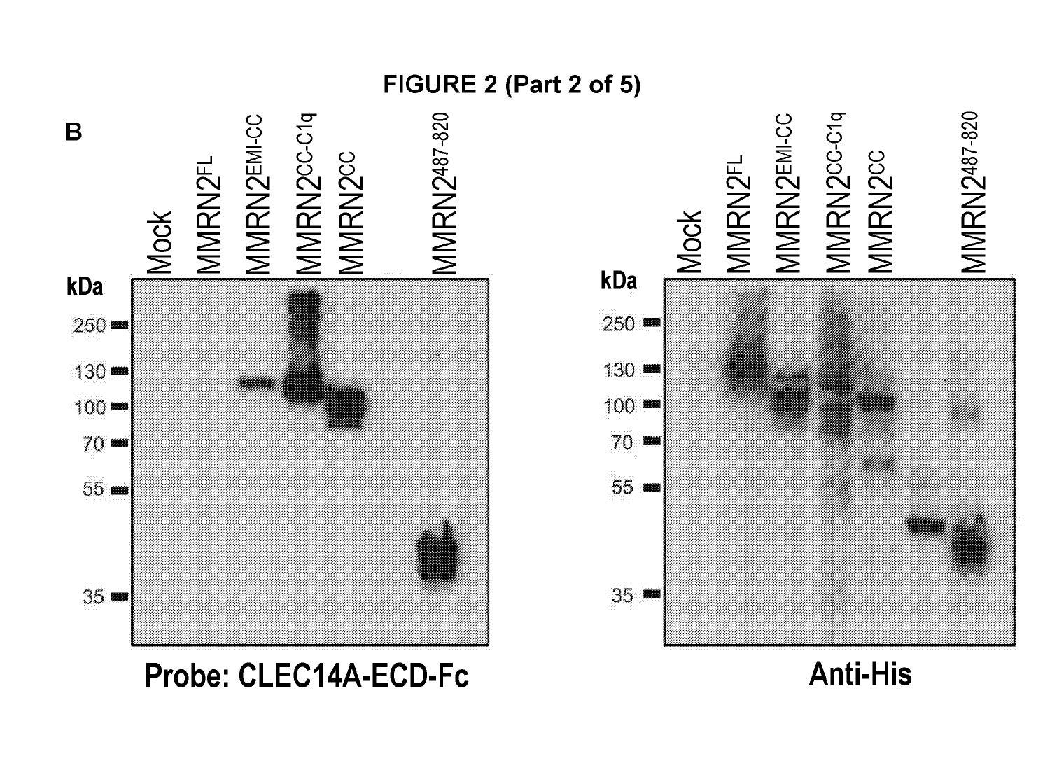

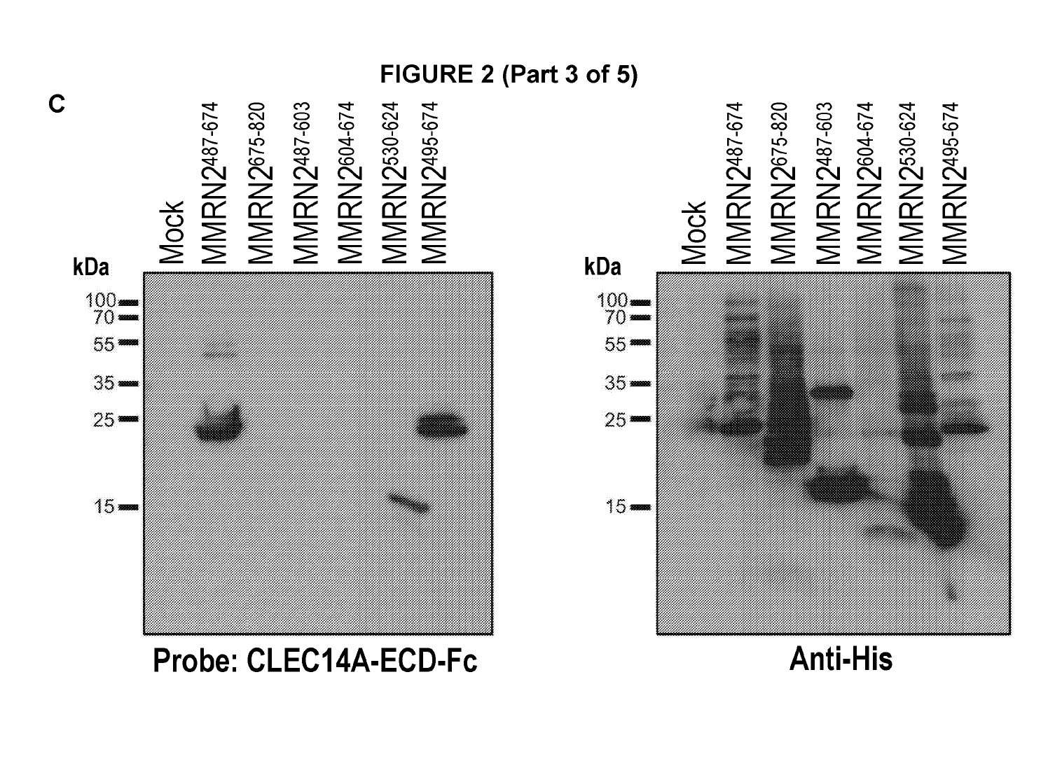

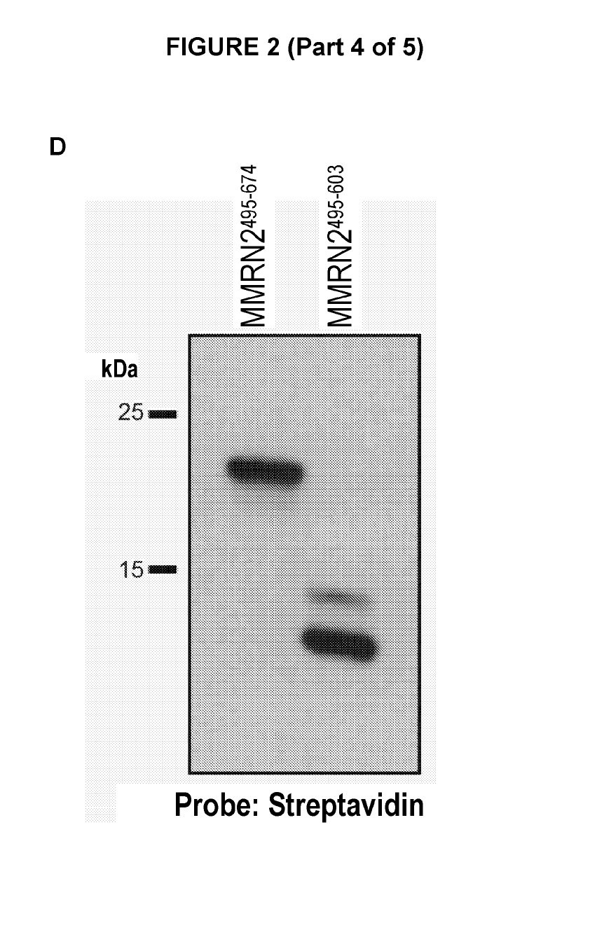

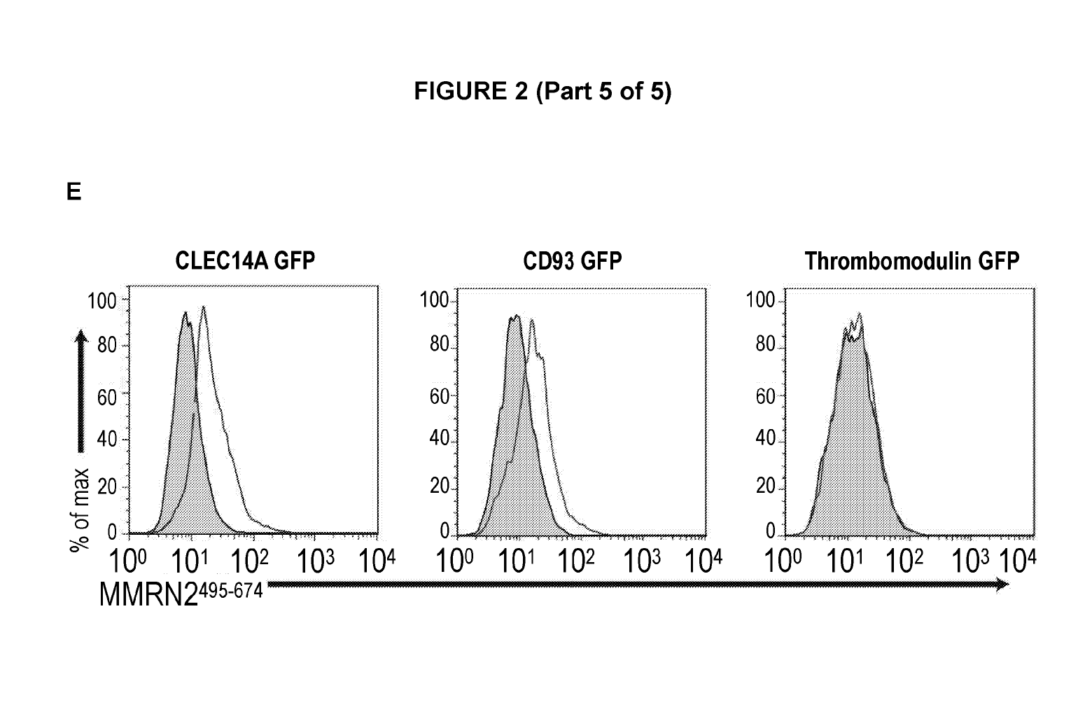

FIG. 2. CLEC14A binds MMRN2 between residues 530-624. (A) Diagrams of MMRN2 truncation mutants, showing the elastin microfibril interface (EMI) domain, coiled-coil (CC) domain and complement related C1q domain. The minimal CLEC14A binding fragment is highlighted in black. (B,C) HEK293T were transfected with truncation mutants. Lysates were far western blotted under reduced conditions with CLEC14A extracellular domain fused to an Fc tag (CLEC14A-ECD-Fc) and western blotted by use of His tag antibodies. (B) The smallest binding fragment was identified as MMRN2⁴⁸⁷⁻⁸²⁰, MMRN2^{FL} fails to bind CLEC14A-ECD-Fc. (C) The smallest MMRN2 fragment binding CLEC14A is MMRN2⁵³⁰⁻⁶²⁴. (D) MMRN2⁴⁹⁵⁻⁶⁷⁴ and MMRN2⁴⁹⁵⁻⁶⁰³ bind to streptavidin under reducing conditions after biotinylation. (E) Flow cytometry histograms of HEK293T transfected with GFP tagged CTLD group 14 family members and stained with biotinylated MMRN2⁴⁹⁵⁻⁶⁷⁴ (grey line), confirming binding to CLEC14A and CD93 but not thrombomodulin. Streptavidin-PE was used as a control for background binding (grey shaded).

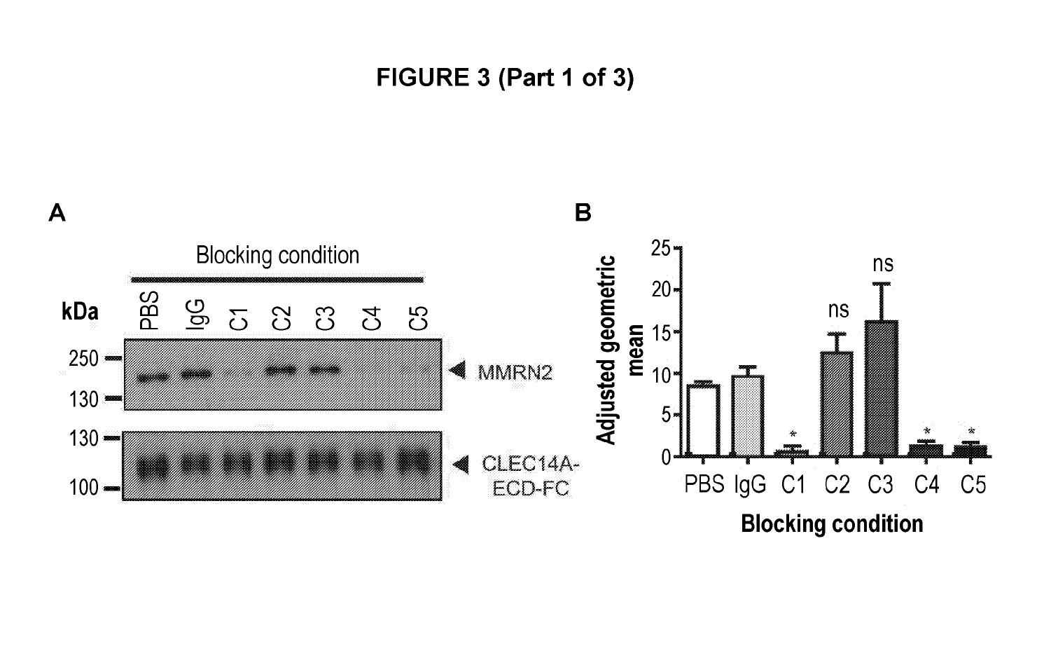

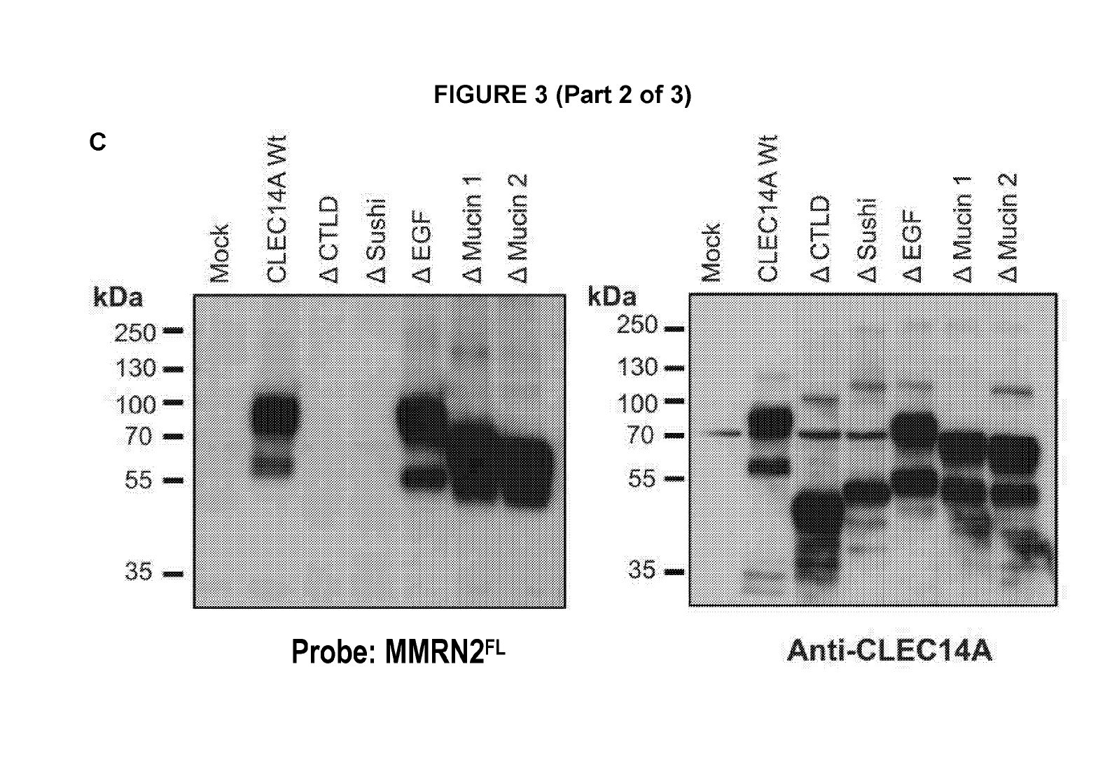

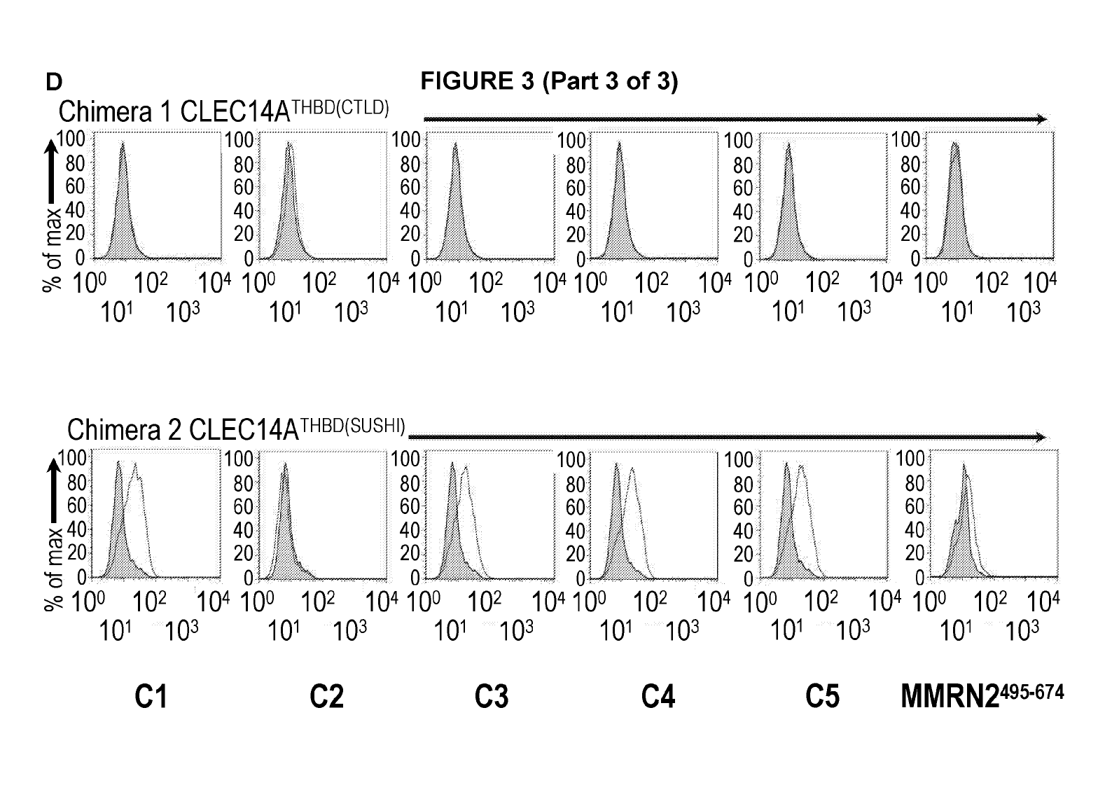

FIG. 3. A distinct region of MMRN2 binds CLEC14A in the C-type lectin domain. (A) CLEC14A-ECD-Fc was used to pull down MMRN2^{FL} from HEK293T transfected lysates. hCLEC14A-ECD-Fc was pre-incubated with either PBS, mouse control IgG, or C1-5. C1, C4 and C5 blocked MMRN2 enrichment whereas C2 and C3 did not. (B) Flow cytometry analysis of CLEC14A-ECD-Fc binding to HUVEC surface. CLEC14A-ECD-Fc was pre-incubated with same conditions as in pull downs. C1, C4 and C5 significantly blocked CLEC14A-ECD binding to HUVEC. Mann-Whitney test *p<0.05 n=4 error bars represent standard error mean (SEM). (C) HEK293T transfected with CLEC14A domain deletions were lysed and far western blotted with MMRN2^{FL} under non-reduced conditions. Upon detection of the MMRN2^{FL} His tag, MMRN2 binding is observed in all mutants except those lacking the CTLD or sushi domains. CLEC14A blot was included to show expression of each mutant protein. (D) Flow cytometry analysis of HEK293T transfected with chimera 1 CLEC14A^{THBD(CTLD)} or chimera 2 CLEC14A^{THBD(sushi)}. None of the anti-CLEC14A antibodies nor the MMRN2⁴⁹⁵⁻⁶⁷⁴ fragment bound to chimera 1 CLEC14A^{THBD(CTLD)} except modest binding with C2. All antibodies except C2 bound to chimera 2 CLEC14A^{THBD(sushi)}. The MMRN2⁴⁹⁵⁻⁶⁷⁴ fragment could also bind chimera 2 CLEC14A^{THBD(sushi)}.

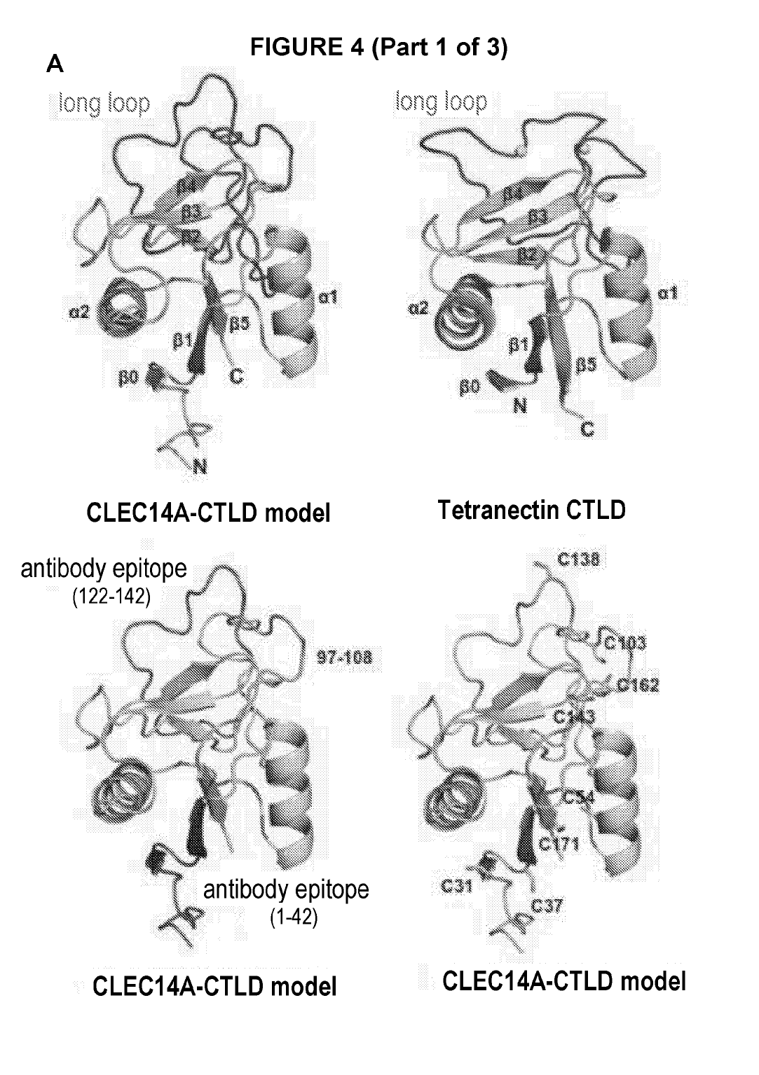

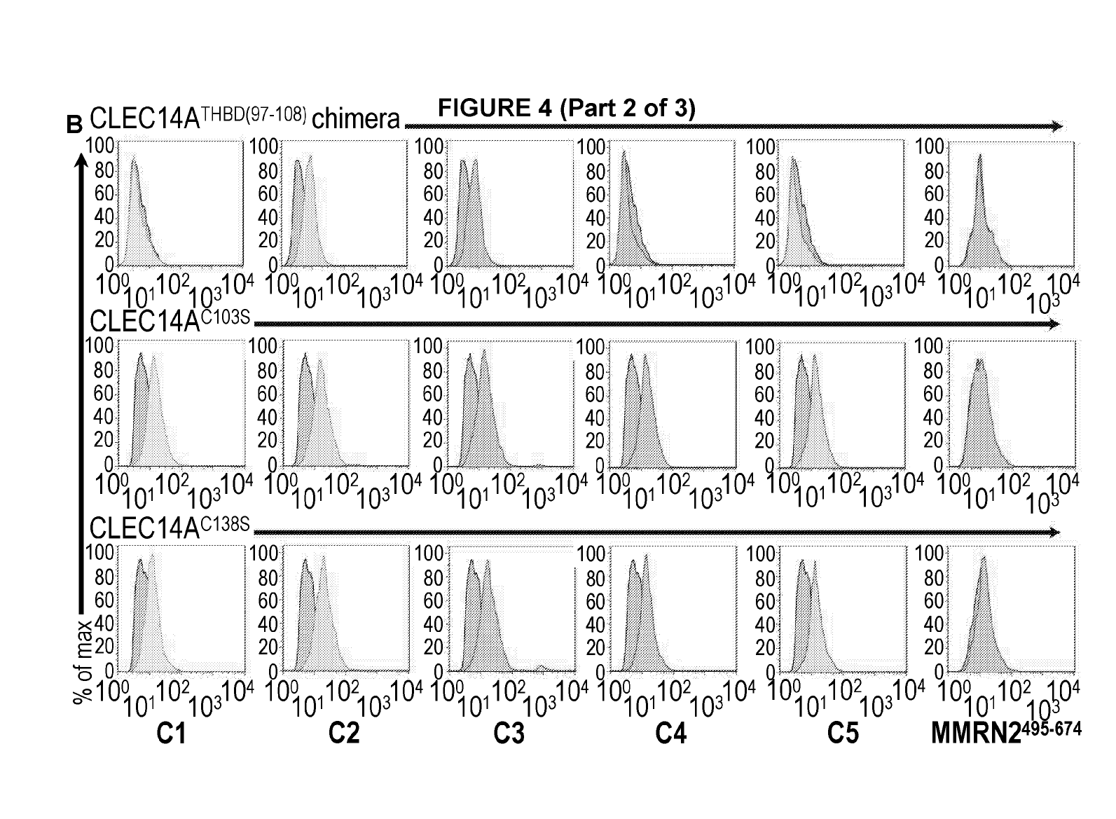

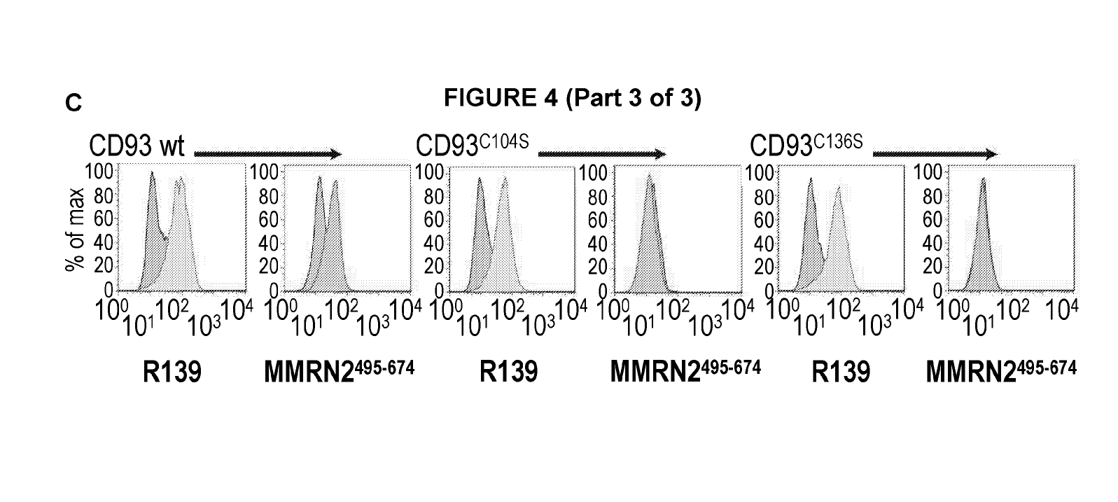

FIG. 4. CLEC14A and CD93 bind MMRN2 in long loop region of the CTLD. (A) CLEC14A-CTLD iTASSER generated molecular model, displaying long loop region in red and numbered α-helices and β-sheets. Solved crystal structure of tetranectin CTLD (1TN3), displaying long loop region in red and numbered a-helices and β-sheets. CLEC14A-CTLD model with predicted antibody epitopes 1-42 (showing 21-42 in the model) (blue), 122-142 (orange) and region 97-108 (grey). CLEC14A-CTLD model displaying predicted arrangement of cysteine residues. Cysteines C31, C37, C54, C143, C162 and C171 are canonical CTLD fold cysteines and are in close proximity to form disulphide bonds. C103 and C138 are non-canonical cysteines present in the long loop region. (B) Flow cytometry analysis of HEK293T transfected with CLEC14A^{THBD(97-108)}, CLEC1 4A^{C103S} or CLEC14A^{C138S}. Only C2 and C3 bind to CLEC14A^{THBD(97-108)}, all C1-5 bind CLEC14A^{C103S} and CLEC14A^{C138S}all CLEC14A mutant proteins fail to bind MMRN2⁴⁹⁵⁻⁶⁷⁴. (C) Flow cytometry analysis of HEK239T transfected with CD93 wild type (wt), CD93^{C104S} or CD93^{C136S}. All proteins bind R139 antibody showing correct conformational folding and presence at the cell surface, although both CD93^{C104S} and CD93^{C136S} fail to bind MMRN2⁴⁹⁵⁻⁶⁷⁴.

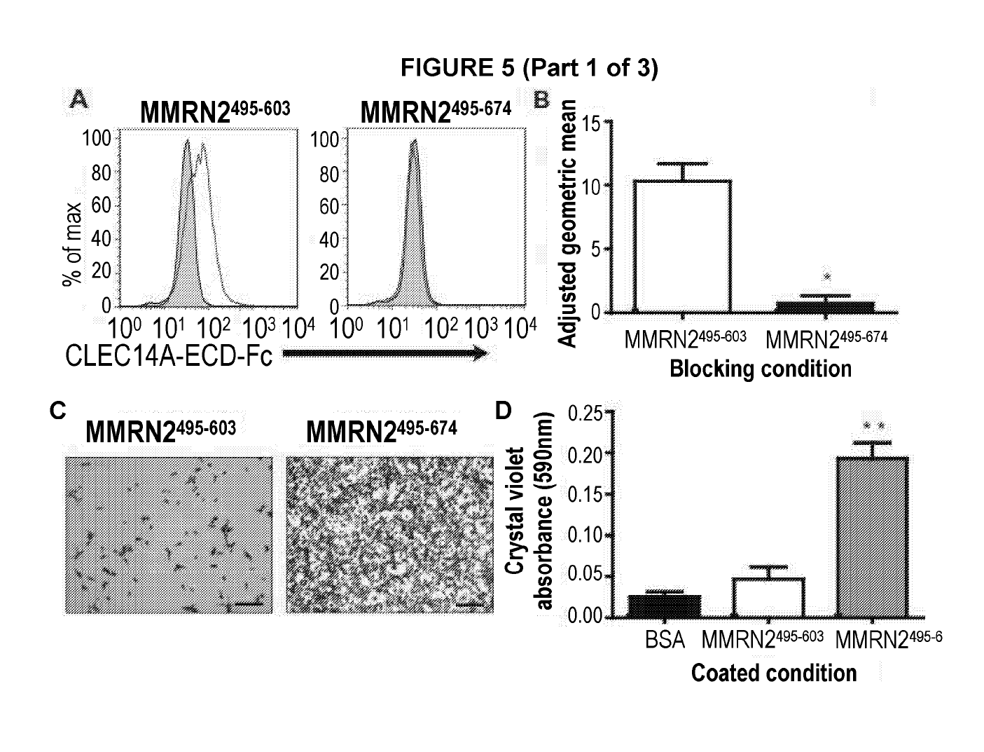

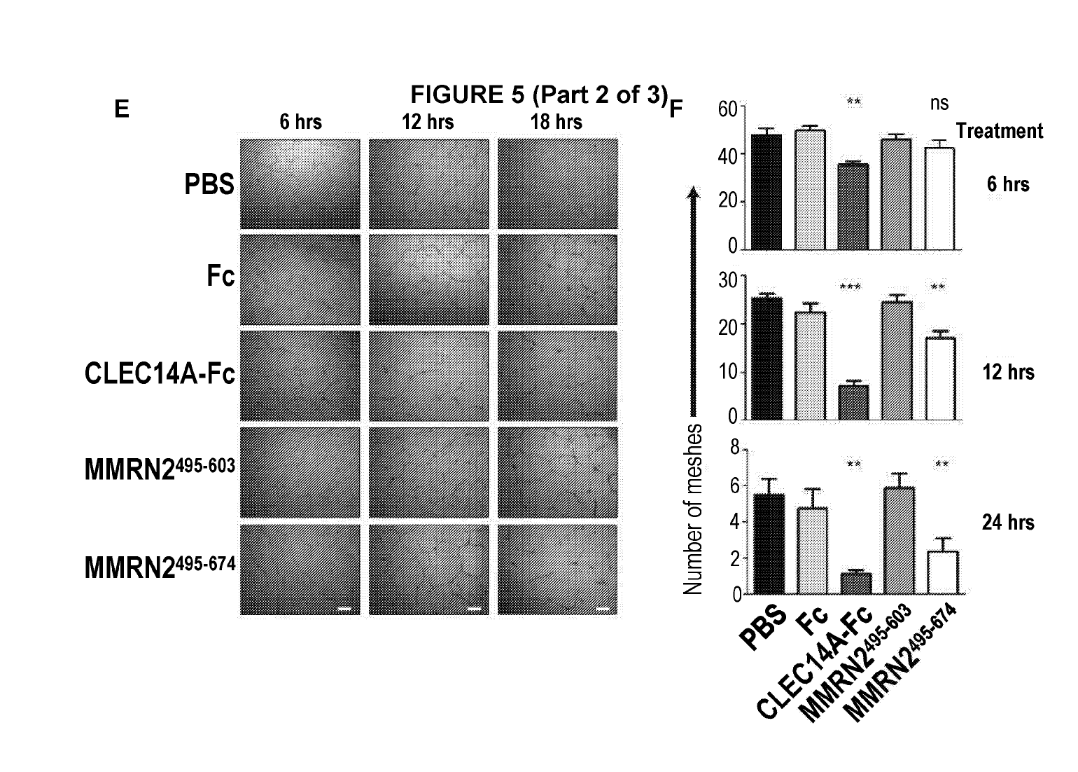

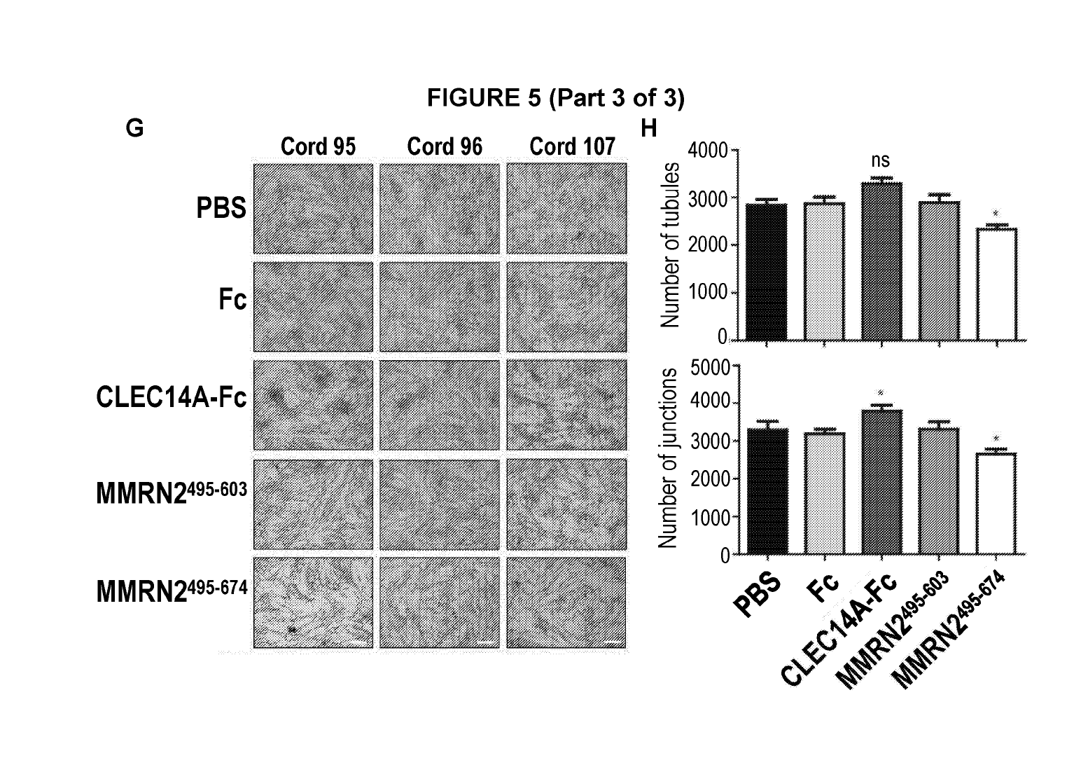

FIG. 5. MMRN2⁴⁹⁵⁻⁶⁷⁴ inhibits angiogenesis in vitro. (A) Representative flow cytometry histograms of CLEC14A-ECD-Fc pre-incubated with MMRN2⁴⁹⁵⁻⁶⁷⁴ or MMRN2⁴⁹⁵⁻⁶⁰³ and then used to stain HUVEC surface. (B) MMRN2⁴⁹⁵⁻⁶⁷⁴ significantly blocked cell surface binding whereas MMRN2⁴⁹⁵⁻⁶⁰³ did not. Mann-Whitney test *p<0.05 n=4. (C) Representative images of MMRN2⁴⁹⁵⁻⁶⁰³ and MMRN2⁴⁹⁵⁻⁶⁷⁴ coated plates with adherent HUVEC, scale bar=150 μm. (D) HUVEC adhered significantly more to MMRN2⁴⁹⁵⁻⁶⁷⁴ compared to MMRN2⁴⁹⁵⁻⁶⁰³ or bovine serum albumin (BSA), Mann-Whitney test **p_{<}0.005 n=6. (E) HUVEC were added to Matrigel and treated with PBS, human Fc, CLEC14A-ECD-Fc (C14A-Fc) MMRN2⁴⁹⁵⁻⁶⁰³ or MMRN2⁴⁹⁵⁻⁶⁷⁴. Representative images from 6, 12 and 18 hours post Matrigel plating from one of three independent experiments, scale bar=100 μm. (F)

Quantification of number of meshes at 6, 12 and 18 hour time points. Representative data from one of three independent experiments. Mann-Whitney test *p<0.05, “p<0.005, ***p<0.0005. Error bars represent standard error mean (SEM). (G) Representative images of HUVEC-fibroblast co-culture assay. Scale bar=800 μm. (H) Quantification of number of tubules and number of junctions. Representative data from one of three independent experiments. Mann-Whitney test *p<0.05. Error bars represent SEM.

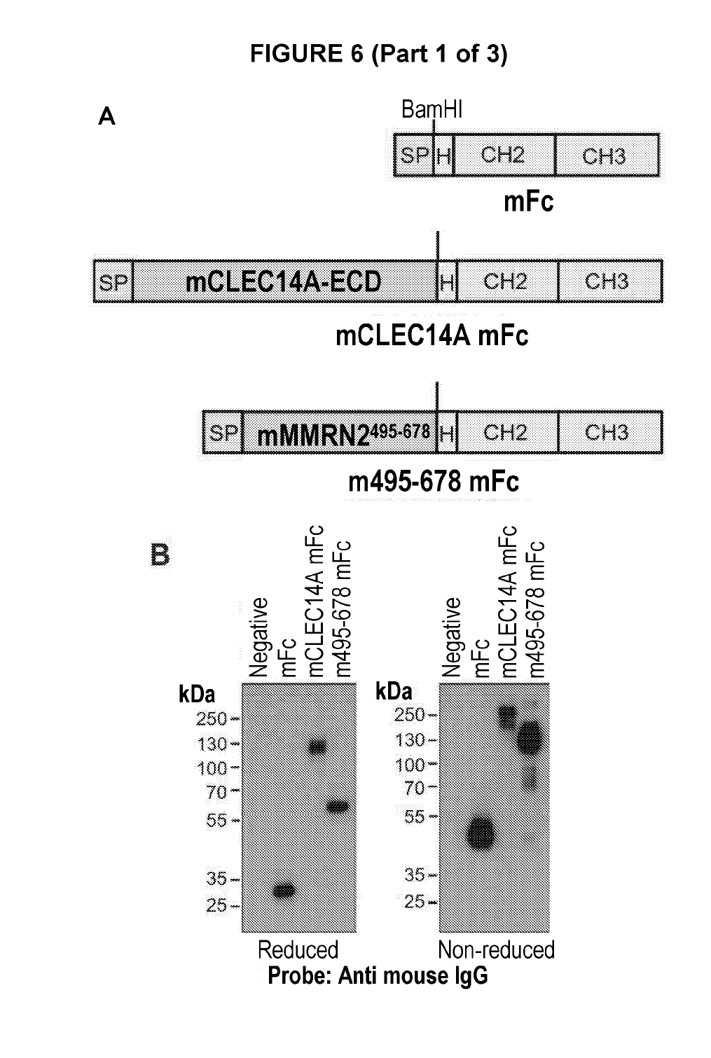

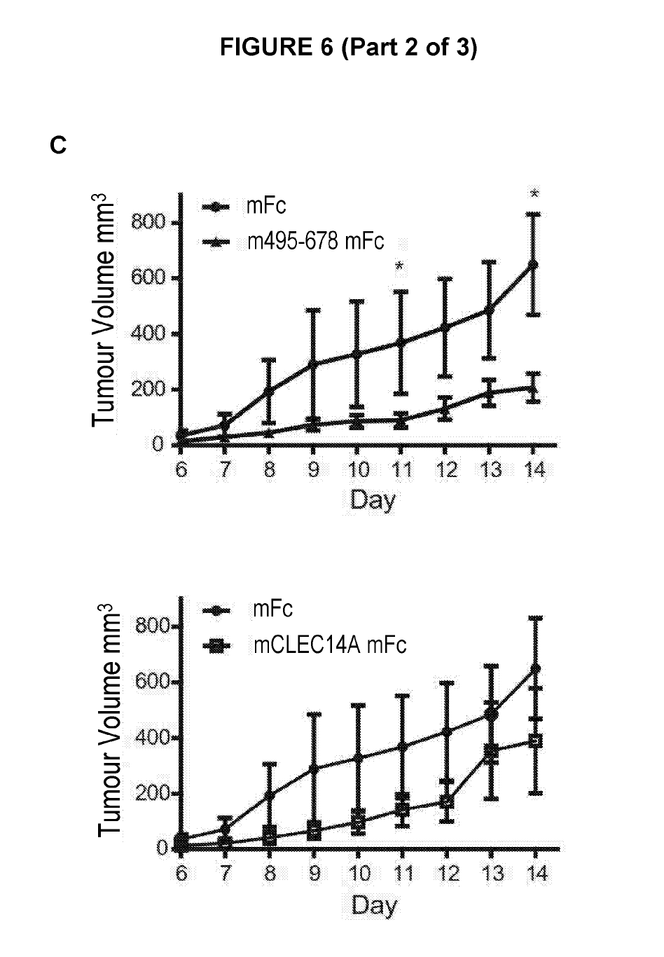

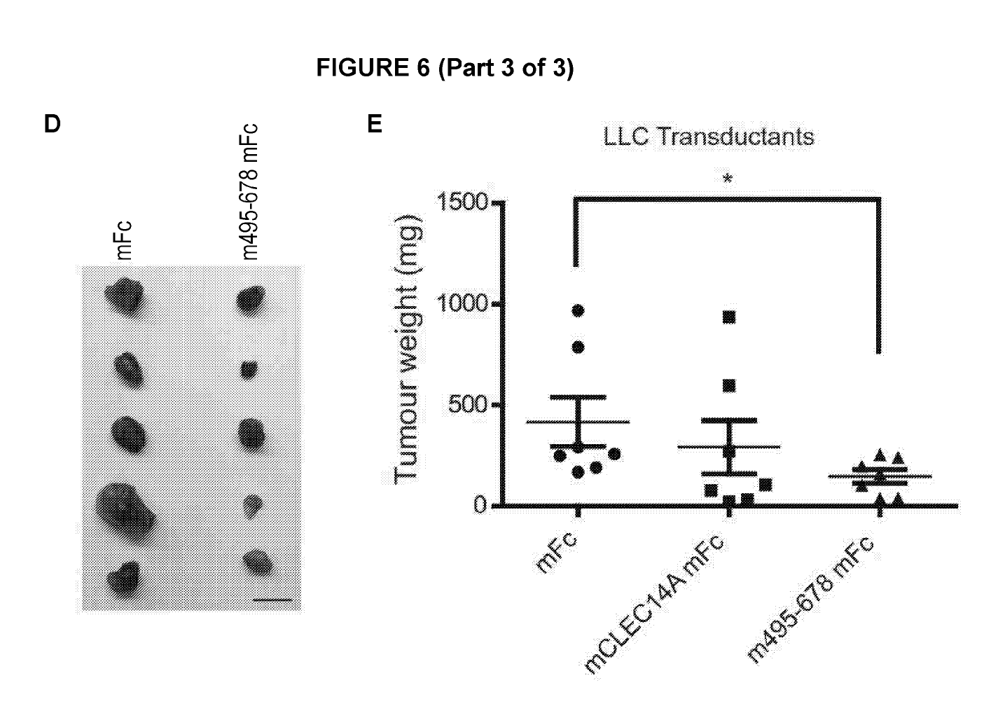

FIG. 6. Mouse MMRN2⁴⁹⁵⁻⁶⁷⁸ inhibits tumour growth. (A) Diagrams of constructs used to transduce Lewis lung carcinoma cells. Mouse hinge region (H), Constant heavy chain 2 and 3 (CH2 and CH3) were fused to mouse CLEC14A signal peptide (SP) to allow secretion of mouse Fc (mFc). Mouse Fc was also fused to mouse CLEC14A-ECD (mC14A-mFc) and mouse MMRN2⁴⁹⁵⁻⁶⁷⁸ (m495-678 mFc). (B) Western blots detecting mouse Fc tag of conditioned media of LLC transduced with each fusion gene. All proteins are secreted and form dimers under non-reduced conditions. (C) Tumour volume was measured from days 6 to 14. Mann-Whitney test *p<0.05 n=7. (D) Representative image of tumours from mFc and m495-678 mFc LLC post excision. Scale bar=10 mm. (E) End-point tumour weight of mFc, mC14A mFc and m495-678 mFc. Mann Whitney test *p<0.05 n=7. Error bars represent SEM.

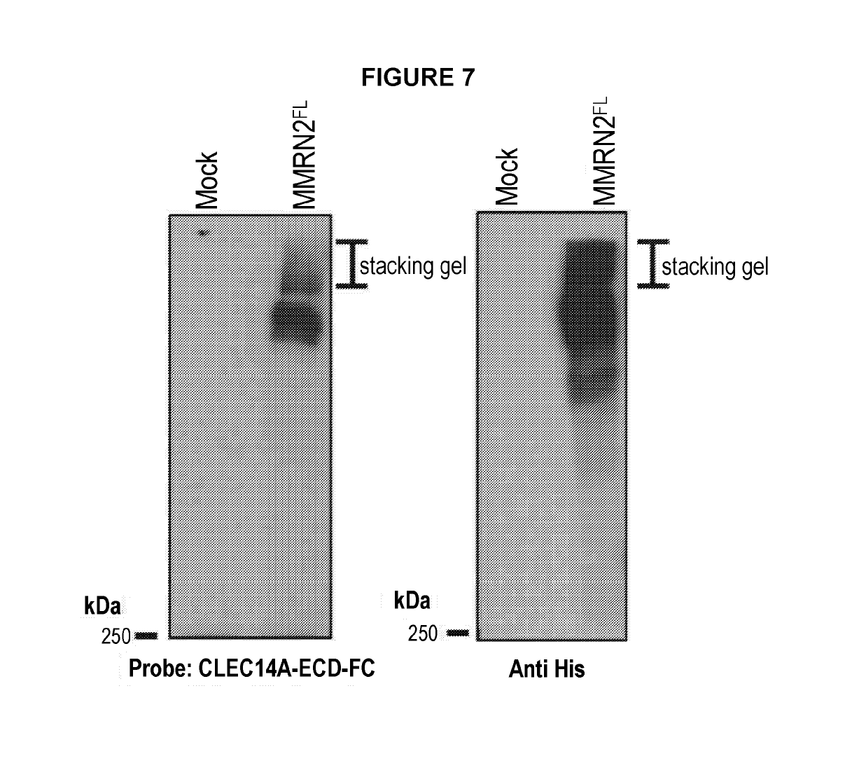

FIG. 7. CLEC14A-ECD binds MMRN2 under non-reduced conditions. HEK293T cells transfected with MMRN2^{FL} were lysed and separated on a 6% polyacrylamide gel. MMRN2^{FL} forms large molecular weight complexes that barely enter the stack gel. CLEC14A far westerns show binding to MMRN2^{FL} multimers. His tag blot is included to show expression of MMRN2^{FL}.

FIG. 8. Amino acid sequence alignments of human MMRN2⁴⁹⁵⁻⁶⁷⁴ and corresponding regions from different species. PRALINE (36) generated amino acid alignments of MMRN2 regions from human (Accession no. Q9H8L6) (SEQ ID NO: 47), bull (E1BJW3) (SEQ ID NO: 48), chimp (H2Q273) (SEQ ID NO: 49), rat (D4ABX6) (SEQ ID NO: 50), mouse (A6H6E2) (SEQ ID NO: 51), chicken (E1BTB7) (SEQ ID NO: 54) and two zebrafish proteins MMRN2a (zebrafish (a)) (F1R6V5) (SEQ ID NO: 52) and MMRN2b (zebrafish (b)) (E7FG77) (SEQ ID NO: 53). This reveals a highly conserved region between residues 588-620 (red line) of human MMRN2. Residues are labelled for conservation by colour. Blue=unconserved, red=conserved.

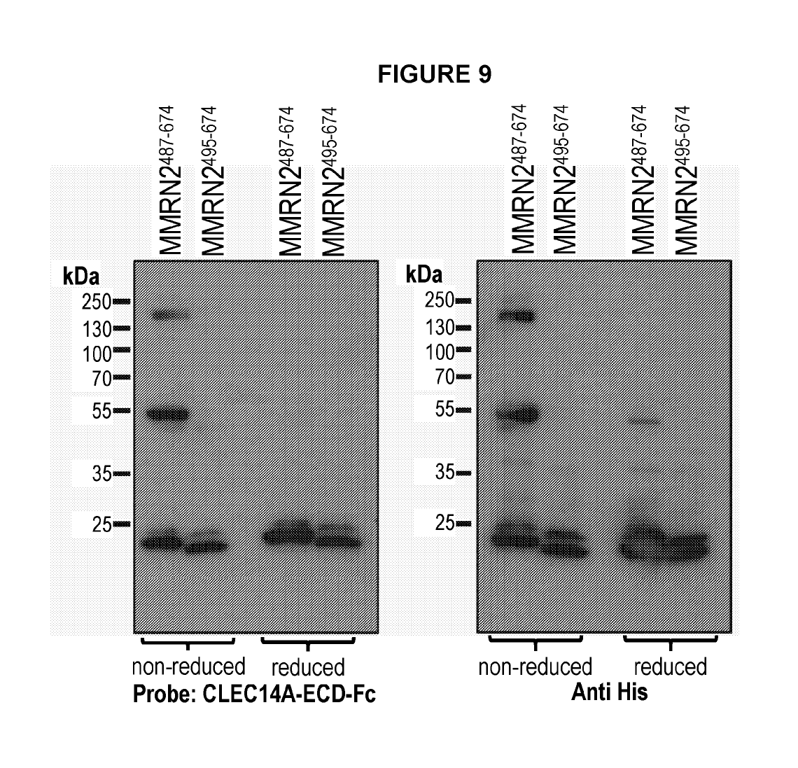

FIG. 9. CLEC14A-ECD-Fc binds MMRN2⁴⁸⁷⁻⁶⁷⁴ and MMRN2⁴⁹⁵⁻⁶⁷⁴. Each MMRN2 fragment was transfected into HEK293T cells, lysed and subjected to far western blotting using CLEC14A-ECD-Fc, this revealed binding to both fragments under reduced (R) and non-reduced (NR) conditions. Under NR conditions MMRN2⁴⁸⁷⁻⁶⁷⁴ forms large molecular weight multimers that also bind CLEC14A. His tag blot included to show expression of each protein.

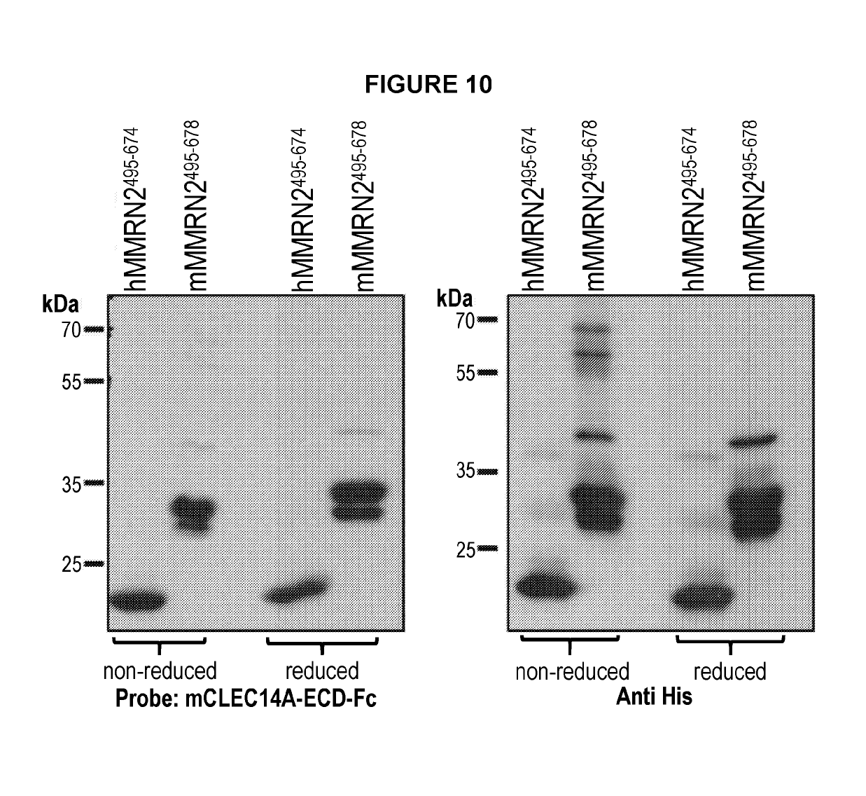

FIG. 10. Mouse CLEC14A directly binds to human MMRN2⁴⁹⁵⁻⁶⁷⁴ and mouse MMRN2⁴⁹⁵⁻⁶⁷⁸. Far western blots using mouse CLEC14A-ECD-Fc shows specific binding to human MMRN2⁴⁹⁵⁻⁶⁷⁴ and equivalent mouse MMRN2⁴⁹⁵⁻⁶⁷⁸, under reduced and non-reduced conditions. His tag blot is included to show expression of each protein fragment.

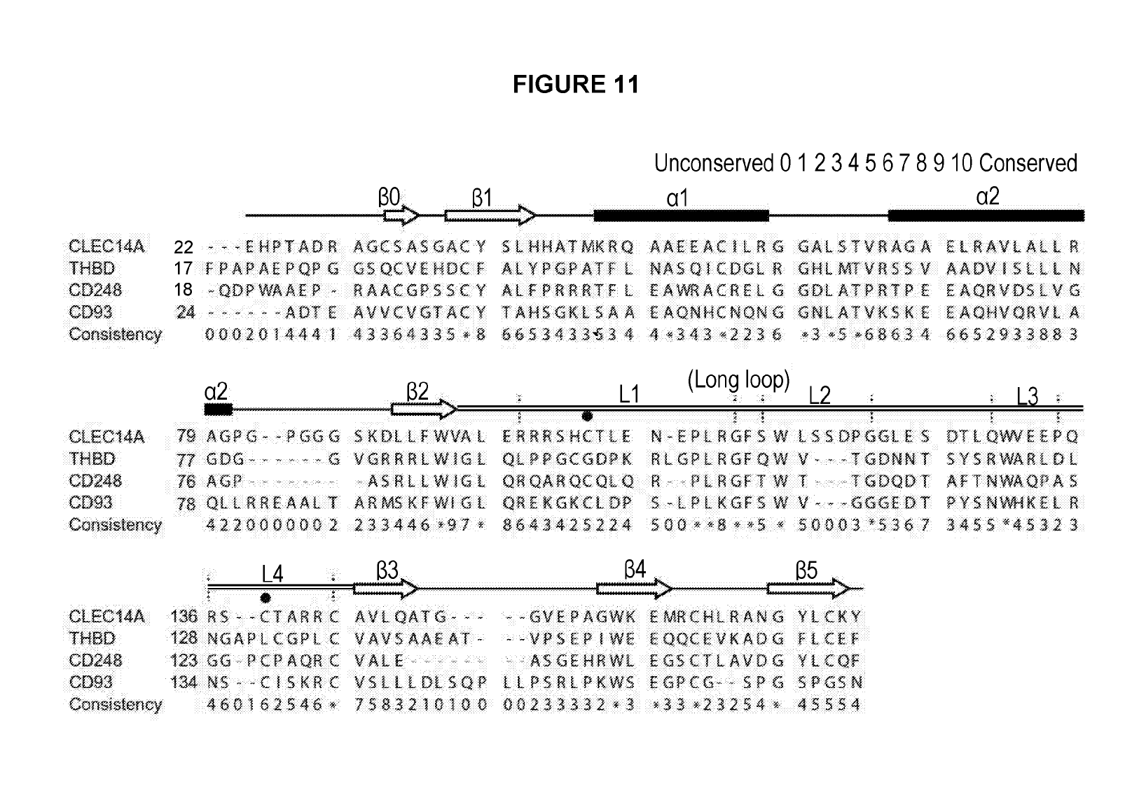

FIG. 11. Amino acid sequence alignments of CTLD group 14 family members. Human CLEC14A (Accession no. Q86T13) (SEQ ID NO: 55), CD93 (Q9NPY3) (SEQ ID NO: 58), THBD (P07204) (SEQ ID NO: 56) and CD248 (Q9HCU0) (SEQ ID NO: 57) CTLD sequences were aligned using PRALINE without inclusion of signal peptides. The α-helices (yellow rectangle) and β-sheets (green arrow) are mapped onto the alignment corresponding to their position within the predicted CLEC14A CTLD model. There are also four predicted loops L1-L4 mapped within the long-loop region (red) as described for tetranectin CTLD. The L1 loop contains the 97-108 region and the non-canonical cysteine 103. The L4 contains the other non-canonical cysteine C138. Each cysteine residue within the long loop region is labelled with a black dot.

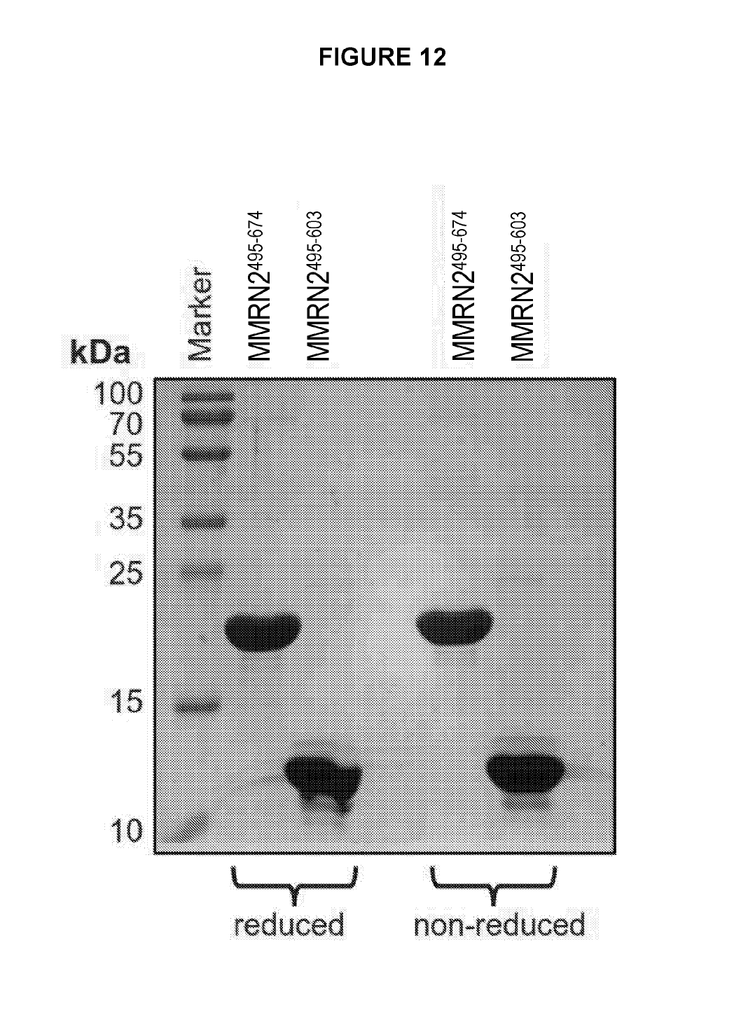

FIG. 12. Coomassie stain of E. coli expressed and purified MMRN2⁴⁹⁵⁻⁶⁷⁴ and MMRN2⁴⁹⁵⁻⁶⁰³. SDS-PAGE and coomassie staining reveals both recombinant protein preparations are of high purity. MMRN2⁴⁹⁵⁻⁶⁷⁴ resolves at ˜20 kDa and MMRN2⁴⁹⁵⁻⁶⁰³ at ˜12.5 kDa.

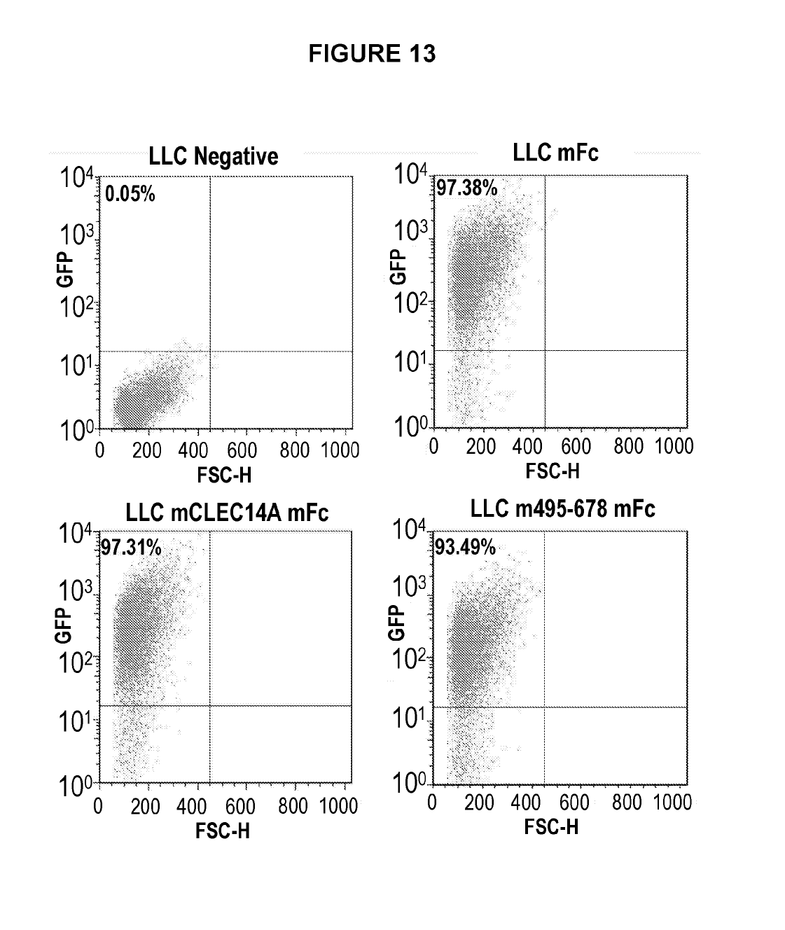

FIG. 13. Flow cytometry analysis of Lewis lung carcinoma transductants. Levels of green fluorescent protein (GFP) were compared to non-transduced cells (LLC −ve). All transduction efficiencies were over 90%.

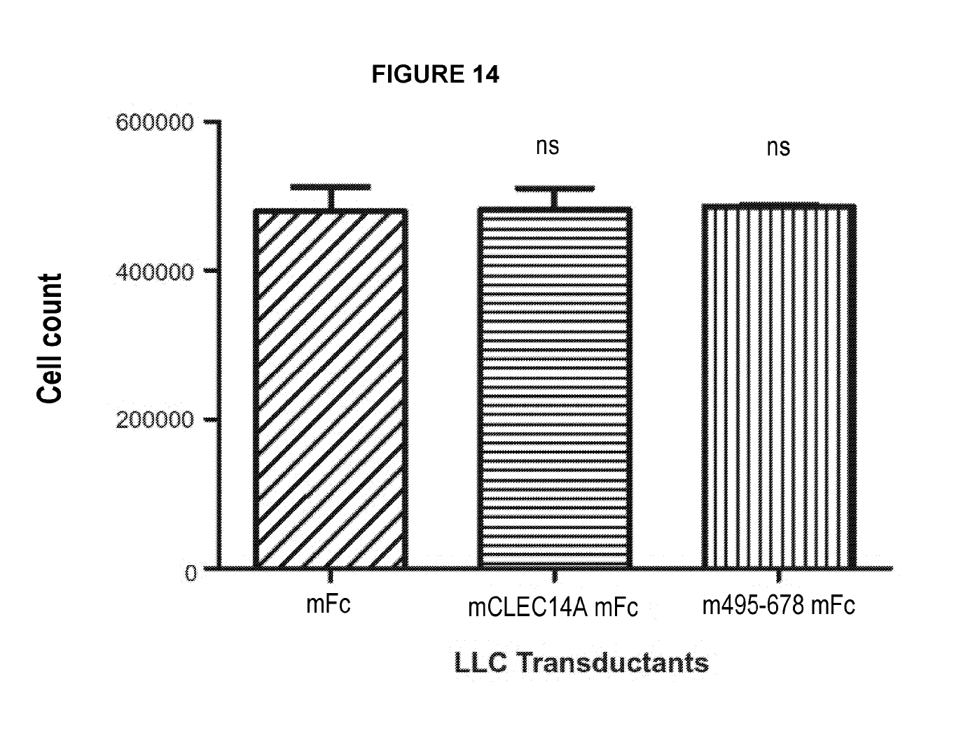

FIG. 14. Cell proliferation is unaffected between Lewis lung carcinoma transductants in vitro. Cells were plated and allowed to proliferate for 2 days and then counted using a coulter counter. This experiment was performed three times. There was no difference in the cell count between each LLC transfectant. Mann Whitney test, mFc vs. mC14A mFc p=1.0, mFc vs. m495-678 p=0.7 n=3. Error bars represent SEM.

FIG. 15. Coding sequence (SEQ ID NO: 59), polypeptide sequence (SEQ ID NO: 60) and amino acid sequence (SEQ ID NO: 61) of human MMRN2.

FIG. 16. Amino acid sequence (SEQ ID NO: 62), cDNA sequence (SEQ ID NO: 63) and coding sequence (SEQ ID NO: 64) of CLEC14A.

FIG. 17. Amino acid sequence (SEQ ID NO: 65), cDNA sequence (SEQ ID NO: 66) and coding sequence (SEQ ID NO: 67) of CD93.

### EXAMPLE 1

### CLEC14A and CD93 Binding Fragment of Multimerin-2 Inhibits Angiogenesis and Tumour Growth

Summary

The C-type lectin domain containing group 14 family members CLEC14A and CD93 are proteins expressed by endothelium and implicated in tumour angiogenesis. Endosialin/CD248 is also a member of this family and is expressed by tumour associated fibroblasts and pericytes. Multimerin-2 (MMRN2) is an endothelial specific extracellular matrix (ECM) protein that is associated with angiogenesis and tumour progression. We show that CLEC14A and CD93 directly bind to MMRN2 whereas the C-type lectin family 14 member thrombomodulin does not. Binding to MMRN2 is dependent on a predicted long loop region in the C-type lectin domain and is diminished by mutations within this domain. We show that CLEC14A and CD93 bind to the same non-glycosylated coiled-coil region of MMRN2. A recombinant peptide of MMRN2 which contains the CLEC14A and CD93 binding region, blocks CLEC14A extracellular domain binding to the endothelial cell surface and increases adherence of HUVEC when the peptide is coated on plates. The MMRN2 peptide is anti-angiogenic in in vitro angiogenesis assays and reduces tumour growth in mouse models. Our findings identify CLEC14A and CD93 interactions with the MMRN2 as targetable components of tumour angiogenesis and growth.

Introduction

Angiogenesis is the growth of new blood vessels from existing ones and is an integral part of normal embryonic development, wound healing and reproduction. It is also a key process in many pathological conditions such as tumourigenesis, diabetic retinopathy and atherosclerosis amongst others (1). The targeting of key molecules involved in angiogenesis offers a strategy for controlling it and a potential for new therapies (2).

The identification of proteins involved in tumour angiogenesis is an essential route to the development of potential anti-cancer agents. The endothelial specific cell surface glycoprotein CLEC14A is known as a tumour endothelial marker, meaning its expression is higher in the vasculature within tumours compared to those in healthy tissue (3). CLEC14A is shed from the cell membrane and has essential roles in endothelial function and angiogenesis (4;5). The related family member CD93 has also been described to be highly expressed in tumour endothelium and recent studies suggest it is a key molecule involved in tumour angiogenesis (6-8).

There is currently little known about the molecular functions of CLEC14A and CD93 in angiogenesis. We and others have previously described CLEC14A as binding to an endothelial specific extracellular matrix (ECM) protein named multimerin-2 (MMRN2) (4;10). The expression of both CLEC14A and MMRN2 is upregulated with tumour progression in two different spontaneous mouse tumour models (10). Antibodies disrupting the CLEC14A-MMRN2 interaction retard angiogenesis and tumour growth, reinforcing the importance of this occurrence (4).

We now show that CD93 as well as CLEC14A can directly bind to MMRN2. Binding of CLEC14A and CD93 has been mapped to a non-glycosylated coiled-coil region of MMRN2. CLEC14A and CD93 interactions are dependent upon the C-type lectin domain and mutations within a predicted binding loop of CLEC14A and CD93 can disrupt interactions with MMRN2. Through this process we have also discovered the epitope for our previously described CLEC14A-MMRN2 blocking antibody C4 (4). A recombinant CLEC14A and CD93 binding fragment of MMRN2 can disrupt in vitro angiogenesis assays and increases adherence of HUVEC. Furthermore, an Fc tagged version of this MMRN2 fragment expressed by Lewis lung carcinoma cells inhibited their growth in vivo but not in vitro. Our findings propose previously unknown protein-protein interactions that occur in endothelium and the surrounding stroma that could provide new targets in anti-angiogenic treatment.

Results

C-Type Lectin Group 14 Family Members CLEC14A and CD93 Directly Bind MMRN2

We previously identified MMRN2 as a binding partner of CLEC14A, one of the members of the C-type lectin domain (CTLD) group 14 family (4). To test whether any of the other CTLD group 14 family members also bind to MMRN2, we employed far western blotting using MMRN2 protein probe as a surrogate for a primary antibody in a standard western blot set up. The CTLD group 14 family members CLEC14A, CD93 and thrombomodulin were all constructed with a C-terminal green fluorescent protein (GFP) tag (FIG. 1A). Constructs were transfected into HEK293T cells and lysates were separated by SDS-PAGE under non-reducing conditions to keep disulphide bonds intact, then transferred to PVDF membranes and blotted using HEK293T lysates overexpressing full-length human MMRN2 with a polyhistidine (His) tag. MMRN2 protein binding was detected by staining with an anti-His tag antibody. This revealed MMRN2 binding to both CLEC14A and CD93 (FIG. 1B). Anti-GFP staining revealed expression of each test protein.

In order to validate the CD93-MMRN2 interaction in endothelial cells, HUVEC lysate was immunoprecipitated with either polyclonal MMRN2 antibodies or the previously validated CD93 monoclonal antibody R139 (13;14). This resulted in enrichment of MMRN2 in CD93 immunoprecipitations (FIG. 10) and enrichment of CD93 in MMRN2 immunoprecipitations (FIG. 1D) but no enrichment in either of the mouse IgG control immunoprecipitations.

CLEC14A and CD93 Bind to a Non-Glycosylated Coiled-Coil Region of MMRN2

MMRN2 is a 949 amino acid glycoprotein that consists of three structural domains, the N-terminal EMI domain, a central coiled-coil domain and a C-terminal C1q domain (15). To further characterise the region of MMRN2 responsible for binding CLEC14A, various MMRN2 constructs were made, each lacking major domains and each including a His tag (FIG. 2A). These included MMRN2 full-length (MMRN2^{FL}), the EMI and coiled-coil (MMRN2^{EMI-CC}), coiled-coil and C1q (MMRN2^{CC-C1q}), coiled-coil domain alone (MMRN2^{CC}) and half of the coiled-coil domain)(MMRN2⁴⁸⁷⁻⁸²⁰). These constructs were transfected into HEK293T cells, lysed and separated by SDS-PAGE under reducing conditions. Upon probing with human CLEC14A extracellular domain fused to an Fc tag (CLEC14A-ECD-Fc) (16), binding was observed in all MMRN2 proteins except MMRN2^{FL} (FIG. 2B). This is surprising as the non-reduced form of MMRN2^{FL} does bind CLEC14A (FIG. 7). Despite this the CLEC14A binding region is clearly within the C-terminal half of the coiled-coil domain of MMRN2.

To further characterise the CLEC14A binding domain of MMRN2 smaller fragments of MMRN2 were constructed. Initially, the MMRN2⁴⁸⁷⁻⁸²⁰ region was further divided in half revealing binding to the MMRN2⁴⁸⁷⁻⁶⁷⁴. Subsequently, this region was further sub-divided and revealed binding of CLEC14A within the MMRN2⁵³⁰⁻⁶²⁴ region but binding was not observed for either of the truncations MMRN2⁴⁸⁷⁻⁶⁰³ or MMRN2⁶⁰⁴⁻⁶⁷⁴ (FIG. 2C). Interestingly, there is a highly conserved region within this portion of MMRN2 (amino acids 588-620) with conservation existing in the two zebrafish orthologues MMRN2a and MMRN2b, suggesting a potential CLEC14A binding motif (FIG. 8). Due to low expression levels and the failure of the MMRN2⁵³⁰⁻⁶²⁴ fragment to be efficiently expressed and purified from HEK293T cells or E. coli, this fragment was not pursued further. We looked to the second smallest fragment MMRN2⁴⁸⁷⁻⁶⁷⁴, as this fragment forms disulphide-linked high molecular weight complexes under non-reduced conditions, which could interfere in downstream assays, the two N-terminal cysteine residues were removed and a new fragment was constructed comprising residues MMRN2⁴⁹⁵⁻⁶⁷⁴. This fragment could still bind to CLEC14A (FIG. 2C and FIG. 9).

To test whether this binding domain existed in mouse, the corresponding regions in mouse MMRN2 (495-678) were also expressed in HEK293T and mouse CLEC14A-ECD-Fc far western blotting showed positive binding (FIG. 10). The human MMRN2⁴⁹⁵⁻⁶⁷⁴ fragment along with the non-CLEC14A binding fragment MMRN2⁴⁹⁵⁻⁶⁰³ was expressed in E. coil with a His tag for purification and a BirA tag for specific biotinylation (17). Biotinylated proteins could bind to streptavidin in western blots (FIG. 2D) and MMRN2⁴⁹⁵⁻⁶⁷⁴ could bind to cell surface expressed CLEC14A and CD93 but not thrombomodulin detected by flow cytometry. This confirmed the CLEC14A and CD93 interactions likely occur in the same region of MMRN2 (FIG. 2E).

Binding to MMRN2 is Dependent on the CTLD of CLEC14A and CD93

We have previously shown that our C4 monoclonal CLEC14A antibody can block interactions with MMRN2 but the C2 antibody cannot, these provide useful tools in determining important regions for CLEC14A-MMRN2 binding (4). To test whether monoclonals C1, C3 or C5 could also exhibit these blocking effects, pull down assays utilising CLEC14A-ECD-Fc blocked with either PBS, mouse IgG control or one of the five CLEC14A antibodies were performed on HEK293T cells overexpressing MMRN2^{FL} protein. This revealed blocking by C1, C4 and C5 monoclonal antibodies but not C2 or C3 (FIG. 3A). These antibodies (C1, C4 and C5) could also block CLEC14A-ECD-Fc from binding to the HUVEC cell surface (FIG. 3B). Furthermore, as these antibodies only bind in flow cytometry and not western blots under reducing conditions they offer a good tool for probing the correct conformational folding of CLEC14A.

To establish which residues of CLEC14A bind to MMRN2, CLEC14A mutant constructs (16) and far western blots were utilised as described for the CTLD group 14 family members. This revealed that CLEC14A lacking the CTLD or sushi domain could no longer bind MMRN2 (FIG. 3C). This was possibly due to the binding region being dependent on both of these regions or CLEC14A cannot fold correctly when lacking one of these domains. To test the latter, chimeric CLEC14A constructs were generated using the CTLD of the non-MMRN2 binding protein thrombomodulin (denoted chimera 1 CLEC14A^{THBD(CTLD)}) and the sushi of thrombomodulin (chimera 2 CLEC14A^{THBD(sushi)}) inserted into the full-length CLEC14A with a GFP tag. Flow cytometry revealed lack of binding of all CLEC14A antibodies to chimera 1 CLEC14A^{THBD(CTLD)} except C2 and positive binding of all antibodies except C2 to chimera 2 CLEC14A^{THBD(sushi)} (FIG. 3D). This confirmed that the chimeric proteins were correctly folded and present on the cell surface. It also suggested that binding epitopes for all anti-CLEC14A antibodies were within the CTLD except for C2. Similarly, the MMRN2⁴⁹⁵⁻⁶⁷⁴ fragment could bind chimera 2 CLEC14A^{THBD(sushi)} but not chimera 1 CLEC14A^{THBD(CTLD)} (FIG. 3d). This confirmed that the CLEC14A CTLD is required for binding to MMRN2.

To ensure that all wild-type and chimeric proteins were expressed at the cell surface, HEK293T cells expressing each were cell surface biotinylated before immunoprecipitation with an anti-GFP antibody. Immunoprecipitates were probed with streptavidin-HRP and this revealed that all chimeras and the wild-type proteins CLEC14A, CD93 and thrombomodulin were expressed at the cell surface.

MMRN2 Binding is Dependent Upon Residues in Long Loop Region of CTLD in CLEC14A and CD93

In order to better understand the CLEC14A CTLD structure and to predict the MMRN2 or antibody epitope recognition surface, a predicted molecular model of the CLEC14A CTLD was generated using the iTASSER server (19). This predicted model exhibited characteristics of the CTLD fold, of a loop in a loop structure with a hydrophobic core (20). The predicted structure also revealed that canonical CTLD cysteine residues C31, C37, C54, C143, C162 and C171 were in close proximity to form disulphide bonds as is the case in many solved structures of CTLDs (FIG. 4A). There are also two non-canonical cysteine residues within the long loop region that are distal in this predicted model (C103 and C138). The predicted model displays a similar overall structure to the X-ray crystal structure of human tetranectin (21) (FIG. 4A).

A recent study demonstrated that CTLD-specific CLEC14A antibodies had similar anti-angiogenic effects as observed with our C4 blocking antibody, we hypothesised that these too could block the CLEC14A-MMRN2 interaction (22). These CTLD specific antibodies have been described to bind epitopes spanning amino acids 1-42 or 122-142 of CLEC14A (23). These regions were mapped onto the predicted CTLD model, revealing 1-42 is proximal to the sushi domain boundary and 122-142 is on the so called “long loop region”. There also existed another region (97-108) which was semi-conserved in CD93 and part of the predicted long loop region (FIGS. 4A and 12). To test whether epitopes for our antibodies or regions important for MMRN2 binding were within these regions, CLEC14A chimeras were generated by swapping with corresponding regions of the non-MMRN2 binding protein thrombomodulin. The 97-108 region spans residues upstream of the hydrophobic core and terminates at residue 109 as this is conserved in all family members and unlikely to be involved in binding. The CLEC14A^{THBD(1-42)} and CLEC14A^{THBD(122-142)} chimeras failed to bind antibodies C1-C5 suggesting they were incorrectly folded (data not shown). In contrast, the CLEC14A^{THBD(97-108)} mutant could bind C2 and C3 but not the MMRN2 interaction blocking antibodies C1, C4 or C5, indicating the binding epitopes for these antibodies are likely within this region. The MMRN2⁴⁹⁵⁻⁶⁷⁴ fragment failed to bind to this CLEC14A^{THBD(97-108)} mutant as expected (FIG. 4B). This long loop region contained the amino acids 97ERRRSHCTLENE108 (SEQ ID NO: 5). To test whether the cysteine residue within the 97-108 region formed disulphide bonds that are important for MMRN2 binding, the mutant CLEC14A^{C103S} was generated along with a mutant of the other non-canonical cysteine (CLEC14A^{C138S}). These mutants could bind all CLEC14A monoclonals C1-C5 but failed to bind to MMRN2⁴⁹⁵⁻⁶⁷⁴ (FIGS. 4B). This highlighted the importance of these residues for CLEC14A-MMRN2 interactions but not CLEC14A antibody binding, suggesting these mutants are correctly folded.

As CD93 also contains two cysteines in the predicted long loop region, the mutants CD93^{C104S} and CD93^{C136S} were generated. The monoclonal R139 anti-CD93 antibody is conformation-sensitive and was used to validate correct folding of CD93 mutants and expression at the cell surface. Both of these mutants along with CD93 wild type (wt) could bind R139 but failed to bind MMRN2⁴⁹⁵⁻⁶⁷⁴ (FIG. 4C). This confirmed the necessity of these cysteines for CD93-MMRN2 interactions.

The CLEC14A and CD93 Binding Fragment of MMRN2 Inhibits Angiogenesis In Vitro

In order to test whether blocking CLEC14A and CD93 interacting with MMRN2 can have effects on angiogenesis; the MMRN2⁴⁹⁵⁻⁶⁷⁴ fragment and the non-binding MMRN2⁴⁹⁵⁻⁶⁰³ fragment were expressed with His tags and purified from E. coli lysates (FIG. 12). As the CLEC14A monoclonal antibodies were shown to inhibit CLEC14A-ECD-Fc binding to the HUVEC cell surface, the same experiment was performed, blocking with MMRN2⁴⁹⁵⁻⁶⁷⁴ or MMRN2⁴⁹⁵⁻⁶⁰³. This resulted in significant blocking with MMRN2⁴⁹⁵⁻⁶⁷⁴ (FIG. 5A and 5B).

MMRN2 has previously been shown to increase HUVEC adherence (24). To test whether the MMRN2⁴⁹⁵⁻⁶⁷⁴ fragment could also have this effect, wells in a 96 well plate were coated with MMRN2⁴⁹⁵⁻⁶⁷⁴, MMRN2⁴⁹⁵⁻⁶⁰³ or BSA as a control. This resulted in HUVEC adherence to MMRN2⁴⁹⁵⁻⁶⁷⁴ but lack of adherence to MMRN2⁴⁹⁵⁻⁶⁰³ or BSA, suggesting an adhesive function for CLEC14A or CD93 or both (FIGS. 5C and 5D).

The MMRN2⁴⁹⁵⁻⁶⁷⁴ and MMRN2⁴⁹⁵⁻⁶⁰³ fragments were examined in angiogenesis assays. As we have previously shown the CLEC14A-ECD-Fc protein to have anti-angiogenic effects (16), this was included in all assays as a positive control, along with a human IgG Fc alone to account for any effects the Fc tag may have. Recombinant proteins were added to HUVEC in Matrigel tube formation assays which resulted in significant decreases in tubule mesh formation with both CLEC14A-ECD-Fc and MMRN2⁴⁹⁵⁻⁶⁷⁴ compared to Fc and MMRN2⁴⁹⁵⁻⁶⁰³ respectively (FIGS. 5E and 5F). The recombinant proteins were then tested in the organotypic human dermal fibroblast-HUVEC co-culture tube formation assay (25). This resulted in modest reductions in tubules and junctions when treated with MMRN2⁴⁹⁵⁻⁶⁷⁴ but in this case not when treated with CLEC14A-ECD-Fc (FIGS. 5G and 5H). Intriguingly, the CLEC14A-ECD-Fc treatments in the co-culture induced formation of knot-like areas with high density of tubules.

The CLEC14A and CD93 Binding Peptide of MMRN2 Reduces Tumour Growth

To test whether disrupting CLEC14A and CD93 interactions had an effect on tumour growth in vivo, the mouse MMRN2⁴⁹⁵⁻⁶⁷⁸ fragment and the mouse CLEC14A-ECD were fused to a mouse IgG Fc tag. These were constructed so as to include the signal peptide of mouse CLEC14A (mCLEC14A) to allow successful secretion along with a murine Fc control (FIG. 6A). LLC were separately lentivirally transduced with these constructs, achieving greater than 90% transduction efficiency (FIG. 13). This Fc-fusion strategy was utilised to increase serum half-life of these expressed proteins and to allow them to be expressed at local sites of neo angiogenesis by cells of mouse origin. Western blots of conditioned media confirmed secretion of fusion proteins (FIG. 6B). The cells were also shown to have no differences in proliferation in vitro (FIG. 14). Transduced LLC were implanted subcutaneously into the flanks of C57BL6 mice and tumour growth was monitored by daily calliper measurements, revealing a slower growth rate in mMMRN2⁴⁹⁵⁻⁶⁷⁸mFc LLC implants compared to mFc (FIG. 6C). Mice were culled after 2 weeks or until the tumour size limit of 1.2 cm³ was reached. Wet weights were analysed revealing a significant reduction in tumour weight in LLC expressing mMMRN2⁴⁹⁵⁻⁶⁷⁸mFc (FIGS. 6D and 6E). There was no significant difference in the weights or growth rates of mCLEC14A-ECD-mFc expressing tumours.

Discussion

CLEC14A and CD93 are emerging as important molecules in angiogenesis, particularly in the tumour setting. Our present study has demonstrated CD93 as being able to bind the CLEC14A ECM ligand MMRN2. These interactions have been dissected and found to involve a predicted loop in the CTLD of CLEC14A and CD93 and regions of MMRN2 within its coiled-coil domain. The CLEC14A and CD93 binding fragment of MMRN2 had anti-angiogenic effects presumably by disrupting normal CLEC14A and CD93 function. We also show that MMRN2 can associate with fibronectin in HUVEC likely dependent on a non-disulphide bonded region of fibronectin, due to binding being observed under reducing conditions.

The observation of the CD93-MMRN2 interaction could explain some observations reported in the literature, such as CD93 CTLD-Fc being able to stain endothelium in human tonsils and descriptions of CD93 being involved in cell adhesion (26;27). Interestingly, expression of CD93 in mouse embryos has been reported to occur at embryonic day 9 and expression of MMRN2 has been observed at embryonic day 9.5, both show expression within the intersomitic vessels (27;28). CLEC14A expression can be detected in brain and intersomitic vessels at embryonic day 10.5 (5), posing a likely role in developmental angiogenesis for MMRN2 and its interactors. The fact that neither CLEC14A nor CD93 knockout mice display gross defects in developmental blood vessel formation suggest the presence of proteins compensating their roles. Double gene knockout of CLEC14A and CD93 may have more dramatic effects on angiogenesis.

A recently described monoclonal antibody recognising CD93 has shown anti-angiogenic properties (29). This antibody epitope has been mapped between the CTLD and sushi domains of CD93. We have shown that this region is not involved in MMRN2 interactions, although it is possible that this antibody indirectly blocks CD93-MMRN2 interactions by CD93 internalisation, alternatively binding of this antibody could sterically hinder CD93 binding to MMRN2. CD93 has also been shown to be important in endothelial migration and tube formation in vitro, phenocopying observations made of CLEC14A (3;5). Furthermore this study showed CD93 deficient mice exhibit defects in tumour angiogenesis in glioblastoma models (7). This too phenocopies anti-tumour effects seen with CLEC14A deficient animals (4), it can be hypothesised that these CLEC14A and CD93 dependent effects are due to them no longer being present to interact with MMRN2. It is interesting to note that more cleaved CD93 is co-immunoprecipitated with MMRN2, suggesting a role for the soluble form. This could explain why less soluble CD93 is detected in colorectal cancer patients, possibly being sequestered by overexpressed MMRN2 in the ECM of the tumour vasculature (8).

We also show that the CLEC14A-ECD can bind to the HUVEC surface, as CLEC14A can be shed at the plasma membrane this provides information on the liberated soluble CLEC14A-ECD. Binding is likely due to MMRN2 being held on the surface, as the MMRN2 interaction blocking antibodies and MMRN2⁴⁹⁵⁻⁶⁷⁴ could inhibit this effect.

In this study we have dissected the molecular characteristics of these interactions, revealing a critical predicted long loop region within both CTLDs of CLEC14A and CD93 where two conserved cysteine residues are essential for interactions with MMRN2. These cysteines are either involved in making direct contacts to MMRN2 or are involved in the tertiary structure of the CTLD long loop. In the predicted structure of the CLEC14A CTLD, cysteine 103 and 138 are distant and unlikely to form disulphide bonds in this model, furthermore as the conformation sensitive anti-CLEC14A antibodies bind to both of these cysteine mutants, disulphide bond formation is unlikely. Nevertheless, while these residues are not important for antibody binding they are vital for binding to MMRN2. These described mutant versions of CLEC14A and CD93 lacking binding to MMRN2 can be tested for other known functions of these proteins.

We hypothesise that critical residues involved in CLEC14A and CD93 binding are present within a highly conserved area of MMRN2. As the murine CLEC14A-ECD fused to a human Fc tag was able to bind both mouse and human MMRN2 fragments, binding sites would need to be fairly conserved, indeed the MMRN2 588-620 region contains 76% homology between human and mouse. Furthermore the non-binding fragment MMRN2⁴⁹⁵⁻⁶⁰³ terminates within this highly conserved region.

We have shown that by interrupting both CLEC14A and CD93 normal functions with the MMRN2⁴⁹⁵⁻⁶⁷⁴ fragment, angiogenesis can be blocked in vitro and tumour growth can be reduced in vivo. This offers a new route to targeting this interaction and antibodies raised against the MMRN2⁴⁹⁵⁻⁶⁷⁴ fragment may have potent anti-angiogenic effects.

The relevance of MMRN2 in angiogenesis has been demonstrated numerous times, with two studies from the same group describing it as an angiostatic molecule, acting by sequestering VE.G.F-A (24;31). However, our studies and those by Zanivan et al. describe MMRN2 as a pro-angiogenic molecule binding to cell surface proteins. These conflicting roles could be context dependent, where CLEC14A and CD93 interactions are separate from those of VE.G.F-A. Our observations of HUVEC adhering to MMRN2⁴⁹⁵⁻⁶⁷⁴ could explain the calcium independent adhesion described for the full-length protein, with CLEC14A, CD93 or both eliciting this adhesive effect (24).

It seems that the related family members CLEC14A and CD93 may be having similar roles in ECM interactions although it is unclear at present whether these interactions have distinct signalling outcomes or whether they have compensatory roles. It is worth mentioning that CLEC14A is upregulated in response to low shear stress, but CD93 is not shear regulated (Bicknell unpublished data). It is possible that each gene is expressed under different circumstances and this explains similarities in binding MMRN2. As CD93 is expressed by other cell types such as haematopoietic cells and platelets, these are also likely to bind MMRN2 and the endothelial ECM. CD93 has recently been found on a subset of non-quiescent leukaemia stem cells and is important for development of acute myeloid leukaemia (32). Future studies will likely shed light on the roles of CLEC14A and CD93 and the signalling of these understudied molecules in angiogenesis.

Materials and Methods

Antibodies and Reagents

Antibodies used: anti-CLEC14A mouse monoclonals C1-C5 were generated in our laboratory (4), anti-CLEC14A sheep polyclonal antisera (R&D Systems, Abingdon, UK #AF4968), anti-polyhistidine tag mouse clone AD1.1.10 (R&D Systems #MAB050), anti-GFP mouse clone 3E1 (Cancer Research UK), anti-MMRN2 polyclonal mouse (Abnova, Taoyuan City, Taiwan/Novus Biologicals, Colorado, USA #H00079812-1301P), anti-CD93 mouse clone R139 (eBioscience, Hatfield, UK #14-0939), anti-CD93 goat polyclonal (R&D Systems #AF2379), anti-CD31 mouse clone JC70A (Dako, Cambridge, UK #M0823), anti-Tubulin mouse clone DM1A (Sigma-Aldrich, Dorset, UK #T9026), anti-fibronectin sheep polyclonal (R&D Systems #AF1918), mouse IgG isotype control (Thermo Scientific, Loughborough, UK #10400C), purified human IgG Fc (Bethyl Laboratories #P80-104) (dialysed against PBS before use in functional assays to remove sodium azide), anti-human IgG Fc peroxidase conjugated (Sigma-Aldrich #A0170), anti-mouse horse radish peroxidase (HRP) conjugated (Dako #P0447), anti-sheep HRP (R&D Systems #HAF016), anti-goat HRP (Dako #P0449), streptavidin HRP conjugated (GE Healthcare #RPN1231) anti-mouse alexafluor 555 (Thermo Scientific #), anti-human IgG Fc FITC conjugated (Sigma-Aldrich #F9512), streptavidin R-phycoerthrin (PE) conjugate (Thermo Scientific #S-866), purified fibronectin (Sigma #F2006).

Plasmid Construction

All type 14 family members, mutants and chimeras were inserted between the EcoRI restriction site in pE.G.FPN1 (Addgene), using the Gibson assembly reaction with a 1:3 vector:insert ratio according to manufacturer's instructions (New England Biolabs), using PCR products by amplification with the following primers CLEC14A wt forward GATCTCGAGCTCAAGCTTCGATGAGGCCGGCGTICGCC (SEQ ID NO: 6), CLEC14A wt reverse TACCGTCGACTGCAGTGCATCACTAGAGCCAAG (SEQ ID NO: 7), CD93 wt forward CGAGCTCAAGCTTCGATGGCCACCTCCATGGGC (SEQ ID NO: 8), CD93 wt reverse TACCGTCGACTGCAGGCAGTCTGTCCCAGGTGTCG (SEQ ID NO: 9), THBD wt forward CGAGCTCAAGCTTCGATGCTTGGGGTCCTGGTC (SEQ ID NO: 10), THBD wt reverse TACCGTCGACTGCAGGAGTCTCTGCGGCGTCCG (SEQ ID NO: 11). For chimeras two PCR products or more were assembled together using the following primers; (chimera 1 CLEC14A^{THBD(CTLD)}; THBD wt forward and THBD-CTLD fused to CLEC14A-sushi reverse CTCAAACTGGAACTCGCAGAGGAAGCC (SEQ ID NO: 12), THBD-CTLD fused to CLEC14A-sushi forward GCGAGTTCCAGTTTGAGGTCTTGTGTC (SEQ ID NO: 13) and CLEC14A wt reverse). (Chimera 2 CLEC14A^{THBD(sushi)}; CLEC14A wt forward and CLEC14A-CTLD fused to THBD-sushi reverse TACCGTCGACTGCAGTGCATCACTAGAGCCAAG (SEQ ID NO: 14), CLEC14A-CTLD fused to THBD-sushi forward GTGCAAGTACCACTTCCCAGCCACCTGCAGGC (SEQ ID NO: 15) and THBD-sushi fused to CLEC14A-E.G.F reverse TCCCGGGGCAAGCGCCCGGCGCCTCCCT (SEQ ID NO: 16), THBD-sushi fused to CLEC14A-E.G.F forward GCCGGGCGCTTGCCCCGGGAGGTACCTC (SEQ ID NO: 17) and CLEC14A wt reverse). (CLEC14A^{C103S}; CLEC14A wt forward and CLEC14A^{C103S} reverse CTCGTTCTCCAGGGTTGAGTGGGAACGCCTGCGCTC (SEQ ID NO: 18), CLEC14A^{C103S} forward GAGCGCAGGCGTTCCCACTCAACCCTGGAGAACGAG (SEQ ID NO: 19) and CLEC14A wt reverse). (CLEC14A^{S138S}; CLEC14A wt forward and CLEC14A^{C138S} reverse CGCGCATCTCCGCGCGGTGGAGGAGCGTTGGGGCTCCTC (SEQ ID NO: 20), CLEC14A^{C138S} forward GAGGAGCCCCAACGCTCCTCCACCGCGCGGAGATGCGCG (SEQ ID NO: 21) and CLEC14A WT reverse).

Human and mouse MMRN2 fragments were amplified from IMAGE clones using the following primers; (MMRN2^{EMI-CC}; MMRN2^{FL} Forward CCGGACCGGTCAGGCTTCCAGTACTAGCC (SEQ ID NO: 22) and MMRN2⁸²⁰ Reverse CTACTAGGTACCCCAGAGCGCCGCGCCC (SEQ ID NO: 23)). (MMRN2^{CC-C1q}; MMRN2¹³³ Forward CCGGACCGGTGATTCCATGGCAATCCCTGA (SEQ ID NO: 24) and MMRN2^{FL} Reverse CGGGGTACCGGTCTTAAACATCAGGAAGC (SEQ ID NO: 25)). (MMRN2^{CC}; MMRN2¹³³ Forward and MMRN2⁸²⁰ reverse). (MMRN2⁴⁸⁷⁻⁸²⁰; MMRN2⁴⁸⁷ Forward CCGGACCGGTTACGTGAAGGACTGCAATTG (SEQ ID NO: 26) and MMRN2⁸²⁰ Reverse), (MMRN2⁴⁸⁷⁻⁶⁷⁴; MMRN2⁴⁸⁷ Forward and MMRN2⁶⁷⁴ Reverse CTACTAGGTACCCGGCCGCGGGGGCTCCG (SEQ ID NO: 27)) (MMRN2⁶⁷⁵⁻⁸²⁰; MMRN2⁶⁷⁵ Forward CCGGACCGGTGCAGAGCACCTGGAGCC (SEQ ID NO: 28) and MMRN2⁸²⁰ Reverse) (MMRN2⁴⁸⁷⁻⁶⁰³; MMRN2⁴⁸⁷ Forward and MMRN2⁶⁰³ Reverse CTACTAGGTACCCGCGTCCTCCAGCAGGG (SEQ ID NO: 29)) (MMRN2⁶⁰⁴⁻⁶⁷⁴; MMRN2⁶⁰⁴ Forward CCGGACCGGTCTGCGGCACGAGGCGGTG (SEQ ID NO: 30) and MMRN2⁶⁷⁴ Reverse) (MMRN2⁵³⁰⁻⁶²⁴; MMRN2⁵³⁰ Forward CCGGACCGGTGGCTCCTCCCTGCAGGCC (SEQ ID NO: 31) and MMRN2⁶²⁴ Reverse CTACTAGGTACCCTCAGACATCTCCTCCAGC (SEQ ID NO: 32)) (MMRN2⁴⁹⁵⁻⁶⁷⁴; MMRN2⁴⁹⁵ Forward TAGTAGACCGGTCAGAAGCTCTATTTAGACCTG (SEQ ID NO: 33) and MMRN2⁶⁷⁴ Reverse). (Mouse MMRN2⁴⁹⁵⁻⁶⁷⁸; mouse MMRN2⁴⁹⁵ Forward CCGGACCGGTCAAAGGGTCAACTCTGACGTG (SEQ ID NO: 34) and mouse MMRN2⁶⁷⁸ Reverse CTACTAGGTACCCAACTGTGGGTGCTGCTCC (SEQ ID NO: 35)). All PCR products were digested with Agel and Kpnl and ligated into mammalian expression vector pHL-Avitag3 containing an N-terminal signal peptide and C-terminal BirA and His tags (33).

Codon optimised versions of MMRN2⁴⁹⁵⁻⁶⁷⁴ and MMRN2⁴⁹⁵⁻⁶⁰³ DNA were synthesised as gBlocks (IDT Technologies, Leuven, Belgium) with ends complementary to pET23a expression vector and inserted in using Gibson assembly reactions in between Ndel and Notl restriction sites. The BirA sequence was used as following GGTGGTGGTCTGAACGATATTITTGAAGCTCAGAAAATCGAATGG (SEQ ID NO: 36).

All mouse Fc fusion proteins were inserted into the lentiviral vector pWPi by Gibson reaction between Pmel restriction sites. Mouse CLEC14A-ECD was amplified with the following primers forward ACTAGCCTCGAGG I I I AAACATGAGGCCAGCGCTTGCC (SEQ ID NO: 37) and reverse CACTCGATGAGGATCCGGAAGAGGTGTCGAAAGTCAGAGAAAC (SEQ ID NO: 38), mouse Fc for fusion to mCLEC14A forward CCTCTTCCGGATCCTCATCGAGTGTGCCCAGGGATTGTGGT (SEQ ID NO: 39) and reverse CTGCAGCCCGTAGTTTTCATTTACCAGGAGAGTGGG (SEQ ID NO: 40). Mouse Fc alone was fused to CLEC14A signal peptide with primers; mouse CLEC14A signal peptide forward AGACTAGCCTCGAGG III AAACATGAGGCCAGCGCTTGC (SEQ ID NO: 41) and mouse CLEC14A signal peptide reverse TGAGGATCCCTCCCCATTCCCTGGCCG (SEQ ID NO: 42), and mouse Fc fused to signal peptide forward AATGGGGAGGGATCCTCATCGAGTGTG (SEQ ID NO: 43) and reverse TCCTGCAGCCCGTAG I I I I CATTTACCAGGAGAGTGG (SEQ ID NO: 44). Mouse MMRN2⁴⁹⁵⁻⁶⁷⁸ was Gibson cloned in between the unique engineered BamHI restriction site separating the signal peptide and the mouse Fc and amplified with primers forward CAGGGAATGGGGAGGGATCCCAAAGGGTCAACTCTGACG (SEQ ID NO: 45) and reverse GGCACACTCGATGAGGATCCCAACTGTGGGTGCTGCTC (SEQ ID NO: 46). Mouse MMRN2 and mouse CLEC14A were amplified from IMAGE clones, mouse IgG Fc was amplified from cDNA of hybridomas expressing anti-CLEC14A monoclonal C3.

Protein Expression and Purification

Human CLEC14A-ECD-Fc was expressed in HEK293T cells and purified as described (4). Human MMRN2⁴⁹⁵⁻⁶⁷⁴ and MMRN2⁴⁹⁵⁻⁶⁰³ with His tag alone or BirA and His tag were expressed in E. coli strain BL21 DE3 pLysS (Prome.g.a, Southampton, UK) by induction with 0.5 mM IPTG at OD₆₀₀ 0.6 and grown at 18° C. overnight. Bacterial pellets were homogenised in buffer containing 50 mM Na₂PO₄ pH7.4 400 mM NaCl, 10% (v/v) glycerol, 50 mM imidazole, 0.5 mM TCEP and complete EDTA-free protease inhibitors (Roche) by high pressure lysis in an Emulsiflex-C3 system (17,000 psi) at 4° C. (Avestin), then loaded onto Nickel-NTA affinity columns (GE Healthcare, Hatfield, UK), fractions were eluted using 500 mM imidazole in homogenisation buffer and then purified by size exclusion chromatography using a superdex 200 column and an AKTA fast protein liquid chromatography machine (GE healthcare) in buffer 20 mM Tris pH8.0, 50 mM NaCl. Upon elution, protein samples were buffer exchanged into PBS and endotoxin removed using high capacity endotoxin removal spin columns (Thermo Scientific). Protein samples were filter sterilised and used in various assays.

Cell Culture and Plasmid Transfections

HUVEC were isolated from fresh umbilical cords collected at the Birmingham Women's to Hospital with informed consent as described previously (3). HUVEC were cultured in medium M199 supplemented with 10% (v/v) foetal bovine serum (FBS) (Life technologies), 4 mM L-glutamine, 90 μg/mL heparin (Sigma-Aldrich) and bovine brain extract was prepared as described (34). HEK293T cells were cultured in Dulbecco's modified Eagle's medium (DMEM) (Sigma-Alrdrich), supplemented with 10% (v/v) FBS and 4 mM L-glutamine. 3×10⁶ HEK293T cells in a 10 cm dish were transfected with 9 pg of DNA and polyethylenimine (PEI) as described (4) and allowed 24-48 hours to express proteins.

Cell Surface Biotinylation

HEK293T (3×10⁵) cells were transfected with various constructs using 1 μg DNA and 4 μg PEI in 6 well plates. The next day cells were washed twice with PBS containing Mg²⁺ and Ca²⁺ and then EZ-Link Sulfo-NHS-Biotin (Thermo Scientific # 21217) was incubated at 1 mg/mL in PBS for 30 minutes, biotinylation reaction was quenched using 100 mM glycine and cells were washed twice more with PBS. Then immunoprecipitations were performed as described below.

Structure Prediction Modelling

The i-TASSER (Iterative Threading ASSEmbly Refinement) server was used to predict the 3D molecular structure of CLEC14A CTLD from residues 21-173 (Accession number Q86T13). The predicted structure with the highest C score (0.05) and an organised structure was chosen.

Lentiviral Transduction

HEK293T cells were PEI transfected as above with pWPI containing gene of interest (4.39 μg), psPax2 (3.29 μg) and pMD2G (1.32 μg). Five 10 cm dishes of HEK293T were allowed to generate lentiviral particles for 24 hours, virus containing media was then concentrated using Corning Spin-X UF concentrators with 5 kDa molecular weight cut off (Sigma-Aldrich) and added to 10⁶ LLC for 48 hours. Transduction efficiency was determined using flow cytometry and untransduced LLC as a control for background fluorescence.

Western Blotting, Far Western Blotting

Whole cell protein lysates were made by incubating with lysis buffer (1% (v/v) NP40, 10 mM Tris pH7.5, 150 mM NaCl and 1 mM EDTA) and pelleting insoluble material. Standard protocols for western blotting were used. Far western blotting involved incubating PVDF membranes for 1 hour with protein of interest hCLEC14A-ECD-Fc (2 μg/mL) or lysates of 6×10⁶ HEK293T cells/mL of lysis buffer transfected with MMRN2 FL with His tag (diluted 1:50). The epitope tag of each probed protein was detected with secondary antibody incubation (anti-His or anti-Fc HRP conjugate) and then tertiary anti-mouse HRP for the case of anti-His.

Immunoprecipitation

For immunoprecipitation (IP) experiments HUVEC were lysed in 200 μL of IP lysis buffer (30 mM Tris pH 7.5, 1% (v/v) Triton-X-100, 10% (v/v) glycerol, 100 mM NaCl, 20 mM NaF, 10 mM KH₂PO₄) with protease inhibitors and phosphatase inhibitors (final concentration 5 mM NaF, 1 mM Na3VO4, 10 mM Na β-glycero phosphate, 1 mM EDTA and 5 mM Na pyrophosphate). Lysates were diluted by adding 200 μL of IP binding buffer (50 mM Tris pH 7.5, 20 mM KCl, 1 mg/mL BSA, 2.5% (v/v) glycerol with protease and phosphatase inhibitors) then 2-5 pg of antibodies or hCLEC14A-ECD-Fc were then added. The same quantity of control antibodies or control Fc was added to a separate IP and all mixtures were incubated on a rotator at 4° C. for 4 hours or overnight. Next 30 μL of protein A (for human Fc) or protein G (for mouse and sheep IgG) sepharose beads (Sigma-Aldrich #P9424 #P3296) were washed three times in PBS and then antibody lysate mixtures were added to beads. This was incubated on a rotator at 4° C. for 4 hours or overnight. Each IP was washed four times with 400 μL of IP wash buffer (100 mM Tris pH 7.5, 200 mM NaCl, 0.5% (v/v) NP40). On the last wash, beads and wash buffer was added to a fresh microcentrifuge tube, to minimise non-specific proteins present in original tube. Proteins were eluted from beads by boiling in SDS reducing buffer and then samples were subjected to SDS-PAGE and western blot.

Flow Cytometry

HUVEC or transfected HEK293T cells were detached from tissue culture plates using cell dissociation buffer or scraped and 5×10⁵ cells were stained with 20 ug/mL of antibody of interest or 20 μg/mL of recombinant protein. In blocking experiments, hCLEC14A-ECD-Fc was incubated with 2× molar excess of antibody or recombinant protein on ice for 1 hour. Flow cytometry buffer (PBS with 0.2% (w/v) BSA and 0.02% (w/v) sodium azide) was used for all wash and incubation steps. Samples were analysed on a FACSCalibur machine (BD Biosciences). In experiments involving overexpression of GFP proteins, highly GFP positive cells were gated and used in analysis. Isotype control staining or human Fc alone was used as background fluorescence and this was subtracted from the geometric mean fluorescence intensity of each sample. The following detection reagents were used; Fc binding; anti-human IgG Fc FITC (1:100), biotinylated proteins; streptavidin-PE (1:100), mouse antibodies; anti-mouse alexafluor 555 (1:100).

Adhesion Assay

MMRN2⁴⁹⁵⁻⁶⁷⁴, MMRN2⁴⁹⁵⁻⁶⁰³ or BSA (2 ug) were coated on 96 well plates overnight in PBS at 37° C. Then blocked in 3% BSA PBS for 1 hour and then dissociated HUVEC (50,000/well) were added and allowed to attach for 4 hours at 37° C. HUVEC were washed 5 times, then fixed with 4% paraformaldehyde and stained with 0.5% crystal violet solution (Sigma-Aldrich). Pictures were taken (Leica DM IL microscope and 2M Xli camera) in the centre of each well and absorbance was measured at 590 nm.

Matrigel Tube Formation and Co-Culture Assay

Matrigel and co-culture assays as described (35), except recombinant proteins were added at 20 μg/mL in PBS.

Mouse Tumour Implantation Assays

10⁶ transduced LLC were subcutaneously injected into the right flank of male C57BL/6 mice aged 8-10 weeks old. After two weeks or when tumour size limit of 1200 mm³ was reached animals were culled, tumours excised and wet weights were determined. Mice were housed at the Birmingham Biomedical Services Unit (Birmingham, UK). All animal experimentation was carried out in accordance with Home Office License number PPL 70/8704 held by RB.

### REFERENCES

(1) Carmeliet P. Angiogenesis in health and disease. Nat Med 2003 June; 9(6):653-60.
(2) Khan K A, Bicknell R. Anti-angiogenic alternatives to VE.G.F blockade. Clin Exp Metastasis 2015 Nov. 30.
(3) Mura M, Swain RK, Zhuang X, Vorschmitt H, Reynolds G, Durant S, et al. Identification and angiogenic role of the novel tumor endothelial marker CLEC14A. Oncogene 2012 Jan. 19; 31(3):293-305.
(4) Noy P J, Lodhia P, Khan K, Zhuang X, Ward D G, Verissimo A R, et al. Blocking CLEC14A-MMRN2 binding inhibits sprouting angiogenesis and tumour growth. Oncogene 2015 Nov. 19; 34(47):5821-31.
(5) Rho S S, Choi H J, Min J K, Lee H W, Park H, Park H, et al. Clec14a is specifically expressed in endothelial cells and mediates cell to cell adhesion. Biochem Biophys Res Commun 2011 Jan. 7; 404(1):103-8.
(6) Masiero M, Simoes F C, Han H D, Snell C, Peterkin T, Bridges E, et al. A core human primary tumor angiogenesis signature identifies the endothelial orphan receptor ELTD1 as a key re.g.ulator of angiogenesis. Cancer Cell 2013 Aug. 12; 24(2):229-41.
(7) Langenkamp E, Zhang L, Lugano R, Huang H, Elhassan T E, Georganaki M, et al. Elevated Expression of the C-Type Lectin CD93 in the Glioblastoma Vasculature Re.g.ulates Cytoskeletal Rearrangements That Enhance Vessel Function and Reduce Host Survival. Cancer Res 2015 Nov. 1; 75(21):4504-16.
(8) Olsen R S, Lindh M, Vorkapic E, Andersson R E, Zar N, Lofgren S, et al. CD93 gene polymorphism is associated with disseminated colorectal cancer. Int J Colorectal Dis 2015 July; 30(7):883-90.
(9) Kontsekova S, Polcicova K, Takacova M, Pastorekova S. Endosialin: molecular and functional links to tumor angiogenesis. Neoplasma 2016 Jan. 17.
(10) Zanivan S, Maione F, Hein M Y, Hernandez-Fernaud J R, Ostasiewicz P, Giraudo E, et al. SILAC-based proteomics of human primary endothelial cell morphogenesis unveils tumor angiogenic markers. Mol Cell Proteomics 2013 December; 12(12):3599-611.
(11) Naylor A J, Azzam E, Smith S, Croft A, Poyser C, Duffield J S, et al. The mesenchymal stem cell marker CD248 (endosialin) is a ne.g.ative re.g.ulator of bone formation in mice. Arthritis Rheum 2012 October; 64(10):3334-43.
(12) Opaysky R, Haviernik P, Jurkovicova D, Garin M T, Copeland N G, Gilbert D J, et al. Molecular characterization of the mouse Tem1/endosialin gene re.g.ulated by cell density in vitro and expressed in normal tissues in vivo. J Biol Chem 2001 Oct. 19; 276(42):38795-807.
(13) Guan E N, Burgess W H, Robinson S L, Goodman E B, McTigue K J, Tenner A J. Phagocytic cell molecules that bind the collagen-like region of C1q. Involvement in the C1q-mediated enhancement of phagocytosis. J Biol Chem 1991 Oct. 25; 266(30):20345-55.
(14) Bohlson S S, Silva R, Fonseca M I, Tenner A J. CD93 is rapidly shed from the surface of human myeloid cells and the soluble form is detected in human plasma. J Immunol 2005 Jul. 15; 175(2):1239-47.
(15) Christian S, Ahorn H, Novatchkova M, Garin-Chesa P, Park J E, Weber G, et al. Molecular cloning and characterization of EndoGlyx-1, an EMILIN-like multisubunit glycoprotein of vascular endothelium. J Biol Chem 2001 Dec. 21; 276(51):48588-95.
(16) Noy P J, Swain R K, Khan K, Lodhia P, Bicknell R. Sprouting angiogenesis is re.g.ulated by shedding of the C-type lectin family 14, member A (CLEC14A) ectodomain, catalyzed by rhomboid-like 2 protein (RHBDL2). FASEB J 2016 Mar. 3.
(17) O'callaghan C A, Byford M F, Wyer J R, Willcox B E, Jakobsen B K, McMichael A J, et al. BirA enzyme: production and application in the study of membrane receptor-ligand interactions by site-specific biotinylation. Anal Biochem 1999 Jan. 1; 266(1):9-15.
(18) Simonavicius N, Ashenden M, van W A, Lax S, Huso D L, Buckley C D, et al. Pericytes promote selective vessel re.g.ression to re.g.ulate vascular patterning. Blood 2012 Aug. 16; 120(7):1516-27.
(19) Yang J, Yan R, Roy A, Xu D, Poisson J, Zhang Y. The I-TASSER Suite: protein structure and function prediction. Nat Methods 2015 January; 12(1):7-8.
(20) Zelensky A N, Gready J E. The C-type lectin-like domain superfamily. FEBS J 2005 December; 272(24):6179-217.
(21) Kastrup J S, Nielsen B B, Rasmussen H, Holtet T L, Graversen J H, Etzerodt M, et al. Structure of the C-type lectin carbohydrate recognition domain of human tetranectin. Acta Crystallogr D Biol Crystallogr 1998 Sep. 1; 54(Pt 5):757-66.
(22) Ki M K, Jeoung M H, Choi J R, Rho S S, Kwon Y G, Shim H, et al. Human antibodies targeting the C-type lectin-like domain of the tumor endothelial cell marker clecl4a re.g.ulate angiogenic properties in vitro. Oncogene 2013 Nov. 28; 32(48):5449-57.
(23) Lee S. M, Ki M. K, Jeoung M H, Choi J. R, inventors; Novel antibody specific for clec14a and uses thereof.WO2013187724 A1. 2013 Dec. 19.
(24) Lorenzon E, Colladel R, Andreuzzi E, Marastoni S, Todaro F, Schiappacassi M, et al. MULTIMERIN2 impairs tumor angiogenesis and growth by interfering with VE.G.F-A/VE.G.FR2 pathway. Oncogene 2012 Jun. 28; 31(26):3136-47.
(25) Donovan D, Brown N J, Bishop E T, Lewis C E. Comparison of three in vitro human ‘angiogenesis’ assays with capillaries formed in vivo. Angiogenesis 2001;4(2):113-21.
(26) McGreal E P, Ikewaki N, Akatsu H, Morgan B P, Gasque P. Human C1qRp is identical with CD93 and the mNI-11 antigen but does not bind C1q. J Immunol 2002 May 15; 168(10):5222-32.
(27) Petrenko O, Beavis A, Klaine M, Kittappa R, Godin I, Lemischka I R. The molecular characterization of the fetal stem cell marker AA4. Immunity 1999 June; 10(6):691-700.
(28) Leimeister C, Steidl C, Schumacher N, Erhard S, Gessler M. Developmental expression and biochemical characterization of Emu family members. Dev Biol 2002 Sep. 15; 249(2):204-18.
(29) Orlandini M, Galvagni F, Bardelli M, Rocchigiani M, Lentucci C, Anselmi F, et al. The characterization of a novel monoclonal antibody against CD93 unveils a new antiangiogenic target. Oncotarget 2014 May 15; 5(9):2750-60.
(30) Zhang M, Bohlson S S, Dy M, Tenner A J. Modulated interaction of the ERM protein, moesin, with CD93. Immunology 2005 May; 115(1):63-73.
(31) Colladel R, Pellicani R, Andreuzzi E, Paulitti A, Tarticchio G, Todaro F, et al. MULTIMERIN2 binds VE.G.F-A primarily via the carbohydrate chains exerting an angiostatic function and impairing tumor growth. Oncotarget 2015 Dec. 9.
(32) Iwasaki M, Liedtke M, Gentles A J, Cleary M L. CD93 Marks a Non-Quiescent Human Leukemia Stem Cell Population and Is Required for Development of MLL-Rearranged Acute Myeloid Leukemia. Cell Stem Cell 2015 Oct. 1; 17(4):412-21.
(33) Aricescu A R, Lu W, Jones E Y. A time- and cost-efficient system for high-level protein production in mammalian cells. Acta Crystallogr D Biol Crystallogr 2006 October; 62(Pt 10):1243-50.
(34) Maciag T, Cerundolo J, Ilsley S, Kelley P R, Forand R. An endothelial cell growth factor from bovine hypothalamus: identification and partial characterization. Proc Natl Acad Sci USA 1979 November; 76(11):5674-8.
(35) Wilson E, Leszczynska K, Poulter N S, Edelmann F, Salisbury V A, Noy P J, et al. RhoJ interacts with the GIT-PIX complex and re.g.ulates focal adhesion disassembly. J Cell Sci 2014 Jul. 15; 127(Pt 14):3039-51.
(36) Simossis V A, Heringa J. PRALINE: a multiple sequence alignment toolbox that inte.g.rates homology-extended and secondary structure information. Nucleic Acids Res 2005 Jul. 1; 33(Web Server issue):W289-W294.

1. A portion of multimerin 2 (MMRN2) or a variant thereof, that inhibits the interaction between CLEC14A and MMRN2.

2. A portion of MMRN2 according to claim 1, wherein the portion binds to CLEC14A, optionally wherein the portion binds to a region of CLEC14A corresponding to the region spanning amino acid residues 97-108 of the human CLEC14A polypeptide.

3. A portion of MMRN2 according to claim 1 or 2, wherein the portion does not bind to a mutant CLEC14A polypeptide in which the cysteine corresponding to cysteine-103 of human CLEC14A is mutated and/or the cysteine corresponding to cysteine-138 of human CLEC14A is mutated.

4. An agent that inhibits the interaction between CD93 and MMRN2, optionally wherein the portion is as defined in any of claims 1-3.

5. An agent according to claim 4, wherein the agent is a polypeptide, a peptide, a polynucleotide, a peptidomimetic, a natural product, a carbohydrate, an aptamer or a small molecule.

6. An agent according to claim 4 or 5, wherein the agent is a portion of MMRN2 or a variant thereof.

7. A portion of MMRN2 according to claim 6, wherein the portion binds to CD93, optionally wherein the portion binds to a region of CD93 corresponding to the region spanning amino acid residues 97-108 of the human CD93 polypeptide.

8. A portion of MMRN2 according to claim 6 or 7, wherein the portion does not bind to a mutant CD93 polypeptide in which the cysteine corresponding to cysteine-104 of human CD93 is mutated and/or the cysteine corresponding to cysteine-136 of human CD93 is mutated.

9. A portion of MMRN2 according to any of claims 1-3, 7 and 8, wherein the portion comprises or consists of the coiled-coil domain of MMRN2, or part thereof.

10. A portion of MMRN2 according to claim 9, wherein the coiled-coil domain of MMRN2 corresponds to amino acid residues 133-820 of human MMRN2.

11. A portion of MMRN2 according to any of claims 1-3 and 7-10, wherein the portion comprises or consists of a region of MMRN2 corresponding to the region spanning amino acid residues 487-820 or 487-674 or 495-678 or 495-674 or 530-624 or 588-620 of human MMRN2, or a part thereof.

12. A portion of MMRN2 according to claim 11, wherein the portion comprises or consists of a region corresponding to the region spanning amino acid residues 495-674 of human MMRN2, or a part thereof.

13. A portion of MMRN2 according to any of claims 1-3 and 7-12, wherein the portion is 400 amino acids in length or less, such as 300 amino acids or less, or 200 amino acids or less, or 100 amino acids or less.

14. A portion of MMRN2 according to any of claims 1-3 and 7-13, wherein the portion comprises the structure
BnX¹[V/L]X²X³LX⁴X⁵X⁶FX⁷X⁸LLX⁹DAX¹⁰RHX¹¹X¹²X¹³LX¹⁴X¹⁵LX¹⁶GEEX¹⁷X¹⁸X¹⁹Jᵣ wherein B is a first chemical moiety, X¹-X¹⁹ are any amino acid, J is a second chemical moiety, n=0 or 1, and r=0 or 1 (Formula I),
optionally wherein:
X¹ is any basic amino acid, such as Glu, Asp, Lys or His;
X² is Arg or Lys or Gln;
X³ is any amino acid;
X⁴ is His or Glu or Ser or Asn;
X⁵ is Ser or Gly or Ala;
X⁶ is Ala or Ser or Thr;
X⁷ is any amino acid;
X⁸ is Ala or Thr or Ser;
X⁹ is Glu or Gln or Asn;
X¹⁰ is Leu or Thr or Val or Met;)
X¹¹ is Glu or Gln or Ser;
X12 is Ala or Asp or Glu;
X¹³ is Val or Ala;
X¹⁴ is Ala or Glu;
X¹⁵ is Ala or Ile or Val;
X¹⁶ is Phe or Leu;
X¹⁷ is Val or Met or Phe;
X18 is Leu or Met or Val or Ile; and
X¹⁹ is Glu or Asp.

15. A portion of MMRN2 according to any of claims 1-3 and 7-14, wherein the portion comprises one or more or all amino acids corresponding to any of the following amino acids according to the numbering of human MMRN2 in FIG. 15: Leu-536, Val-575, Leu-578, Val-589, Leu-592, Phe-596, Leu-599, Leu-600, Asp-602, Ala-603, Arg-605, His-606, Leu-610, Leu-613, Gly-615, Glu-616, Glu-617, and Leu-658.

16. A portion of MMRN2 according to any of claims 1-3 and 7-15, wherein the portion comprises one or more or all amino acids corresponding to any of the following amino acids according to the numbering of human MMRN2 in FIG. 15: Leu-497, Glu-506, Gln-527, Val-540, Ala-546, Val-609, Glu-620, Gln-636, Ile-637, Leu-641, Leu-648 and Glu-666.

17. A portion of MMRN2 according to any of claims 1-3 and 7-16, wherein the portion has at least 50% sequence identity to the region spanning amino acid residues 495-674 of human MMRN2.

18. A portion of MMRN2 according to any of claims 1-3 and 7-17, wherein the portion comprises or consists of the amino acid sequence of any of the MMRN2 portions listed in FIG. 8, or any part or variant of said portions.

19. A portion of MMRN2 according to any of claims 1-3 and 7-18, wherein the portion inhibits angiogenesis in an angiogenesis assay, optionally wherein the angiogenesis assay is an aortic ring assay, a sponge angiogenesis assay, an assay of endothelial cell proliferation, an assay of endothelial cell migration and/or an assay of endothelial cell invasion.

20. A portion of MMRN2 according to any of claims 1-3 and 7-19, wherein the portion inhibits tumour growth in an assay of tumour growth.

21. A portion of MMRN2 according to any of claims 1-3 and 7-20, wherein the portion comprises a stabilising moiety at one or both termini.

22. A portion of MMRN2 according to claim 21, wherein the stabilising moiety is any of a amido, acetyl, benzyl, phenyl, tosyl, alkoxycarbonyl, alkyl carbonyl, or benzyloxycarbonyl moiety.

23. A portion of MMRN2 according to any of claims 1-3 and 7-22, wherein the portion is a portion of a variant of MMRN2.

24. A portion of MMRN2 according to claim 23, wherein the variant of MMRN2 has at least 30% sequence identity to the amino acid sequence of human MMRN2.

25. A fusion protein comprising a portion of MMRN2 according to any of claims 1-3 and 7-24, wherein the fusion protein does not comprise wild type MMRN2.

26. An antibody that selectively binds to a portion of MMRN2 according to any of claims 1-3 and 7-24.

27. An antibody according to claim 26, wherein the antibody selectively binds to the coiled-coil domain of MMRN2, or part thereof, optionally wherein the coiled-coil domain of MMRN2 corresponds to amino acid residues 133-820 of human MMRN2.

28. An antibody according to claim 27, wherein the antibody selectively binds to a region of MMRN2 corresponding to the region spanning amino acid residues 487-820 or 487-674 or 495-678 or 495-674 or 530-624 or 588-620 of human MMRN2, or a part thereof.

29. An antibody according to claim 28, wherein the antibody selectively binds to a region corresponding to the region spanning amino acid residues 495-674 of human MMRN2, or a part thereof.

30. A nucleic acid molecule encoding the portion of MMRN2 of any of claims 1-3 and 7-24, or the fusion protein of claim 25, or the antibody of any of any of claims 26-29.

31. A vector, such as an expression vector, comprising the nucleic acid molecule of claim 30.

32. A host cell comprising the nucleic acid molecule of claim 30 or the vector of claim 31.

33. A compound comprising (i) a portion of MMRN2 according to any of claims 1-3 and 7-24 and (ii) a detectable moiety.

34. A compound according to claim 33, wherein the detectable moiety comprises an enzyme, a radioactive atom, a fluorescent moiety, a chemiluminescent moiety or a bioluminescent moiety.

35. A compound according to claim 34, wherein the detectable moiety comprises an affinity tag, such as a histidine tag or an Fc tag or a BirA tag.

36. A compound comprising (i) a portion of MMRN2 according to any of claims 1-3 and 7-24, and (ii) a cytotoxic moiety.

37. A compound according to claim 36 wherein the cytotoxic moiety is selected from a directly cytotoxic chemotherapeutic agent, a directly cytotoxic polypeptide, a moiety which is able to convert a prodrug into a cytotoxic drug, a radiosensitizer, a directly cytotoxic nucleic acid, an antibody (eg an antibody that binds to a cytotoxic immune cell such as a T cell) a nucleic acid molecule that encodes a directly or indirectly cytotoxic polypeptide, or a radioactive atom

38. A compound according to claim 37 wherein the radioactive atom is phosphorus-32, iodine-125, iodine-131, indium-111, rhenium-186, rhenium-188 or yttrium-90.

39. A chimeric antigen receptor (CAR) comprising (a) a portion of MIVIRN2 or a variant thereof that binds to CLEC14A and/or CD93; (b) a transmembrane domain; and (c) an intracellular signalling domain.

40. A CAR according to claim 39, wherein the portion of MMRN2 is as defined in any of claims 3 and 7-24.

41. A CAR according to claim 39 or 40, wherein the transmembrane domain comprises the transmembrane domain of a protein, optionally wherein the transmembrane domain of the protein is selected from the group consisting of the alpha, beta or zeta chain of the T-cell receptor, CD28, CD3 epsilon, CD8, CD45 and CD4.

42. A CAR according to any of claims 39-41, wherein the portion of MMRN2 is connected to the transmembrane domain by a hinge region.

43. A CAR according to any of claims 39-42, wherein the intracellular signalling domain comprises one or more immunoreceptor tyrosine-based activation motifs (ITAMs).

44. A CAR according to any of claims 39-43, wherein the intracellular signalling domain comprises a signalling domain of CD3 zeta, Fc receptor gamma, Fc receptor beta, CD3 gamma, CD3 delta, CD3 epsilon, CD5, CD22, CD79a, CD79b and CD66d.

45. A CAR according to any of claims 39-44, wherein the CAR further comprises one or more costimulatory domains.

46. A CAR according to claim 45, wherein the costimulatory domain is a functional signalling domain obtained from a protein selected from the group consisting of CD28, 41BB, OX40, ICOS and DAP10.

47. A CAR according to any of claim 39-46, wherein the intracellular portion of the CAR comprising the signalling domain of CD3 zeta and the signalling domain of CD28.

48. A CAR according to any of claims 39-47, wherein the CAR further comprises a leader sequence.

49. A CAR according to claim 48, wherein the leader sequence comprises the oncostatin M leader sequence MGVLLTQRTLLSLVLALLFPSMAS.

50. A nucleic acid molecule encoding the CAR of any of claims 39-49.

51. A vector comprising a nucleic acid molecule of claim 50.

52. A cell comprising the nucleic acid molecule of claim 50 or the vector of claim 51.

53. A cell comprising a CAR according to any of claims 39-49.

54. A method of producing a cell comprising introducing a nucleic acid molecule of claim 50 or the vector of claim 51 into a cell.

55. A cell according to claim 52 or 53 or a method according to claim 54, wherein the cell is a T cell or natural killer cell.

56. A pharmaceutical composition comprising an MMRN2 portion according to any of claims 1-3 and 7-24, an agent according to any of claims 4-6, a fusion protein according to claim 25, an antibody according to any of claims 26-29, a nucleic acid molecule according to claim 30 or 50, a vector according to claim 31 or 51, a cell according to any of claims 32, 52, 53 and 55, a compound according to any of claims 33-38, or a CAR according to any of claims 39-49; and a pharmaceutically acceptable diluent, carrier or excipient.

57. A composition comprising an MMRN2 portion according to any of claims 1-3 and 7-24, an agent according to any of claims 4-6, a fusion protein according to claim 25, an antibody according to any of claims 26-29, a nucleic acid molecule according to claim 30 or 50, a vector according to claim 31 or 51, a cell according to any of claims 32, 52, 53 and 55, a compound according to any of claims 33-38, or a CAR according to any of claims 39-49, which composition further comprises at least one additional anti-cancer agent and/or at least one additional anti-angiogenic agent.

58. An MMRN2 portion according to any of claims 1-3 and 7-24, an agent according to any of claims 4-6, a fusion protein according to claim 25, an antibody according to any of claims 26-29, a nucleic acid molecule according to claim 30 or 50, a vector according to claim 31 or 51, a cell according to any of claims 32, 52, 53 and 55, a compound according to any of claims 33-37, or a CAR according to any of claims 39-49; for use in medicine.

59. A method of inhibiting angiogenesis in an individual, the method comprising administering to the individual an MMRN2 portion according to any of claims 1-3 and 7-24, an agent according to any of claims 4-6, a fusion protein according to claim 25, an antibody according to any of claims 26-29, a nucleic acid molecule according to claim 30 or 50, a vector according to claim 31 or 51, a cell according to any of claims 32, 52, 53 and 55, a compound according to any of claims 33-38, or a CAR according to any of claims 39-49.

60. An MMRN2 portion according to any of claims 1-3 and 7-24, an agent according to any of claims 4-6, a fusion protein according to claim 25, an antibody according to any of claims 26-29, a nucleic acid molecule according to claim 30 or 50, a vector according to claim 31 or 51, a cell according to any of claims 32, 52, 53 and 55, a compound according to any of claims 33-38, or a CAR according to any of claims 39-49; for use in inhibiting angiogenesis in an individual.

61. Use of an MMRN2 portion according to any of claims 1-3 and 7-24, an agent according to any of claims 4-6, a fusion protein according to claim 25, an antibody according to any of claims 26-29, a nucleic acid molecule according to claim 30 or 50, a vector according to claim 31 or 51, a cell according to any of claims 32, 52, 53 and 55, a compound according to any of claims 33-38, or a CAR according to any of claims 39-49, in the preparation of a medicament for inhibiting angiogenesis in an individual.

62. A method of combating a disease or condition in an individual, selected from the group consisting of cancer, psoriasis, menorrhagia, endometriosis, arthritis (both inflammatory and rheumatoid), macular degeneration, Paget's disease, retinopathy and its vascular complications (including proliferative and of prematurity, and diabetic retinopathy), benign vascular proliferations, fibroses, obesity and inflammation, the method comprising administering to the individual an MMRN2 portion according to any of claims 1-3 and 7-24, an agent according to any of claims 4-6, a fusion protein according to claim 25, an antibody according to any of claims 26-29, a nucleic acid molecule according to claim 30 or 50, a vector according to claim 31 or 51, a cell according to any of claims 32, 52, 53 and 55, a compound according to any of claims 33-38, or a CAR according to any of claims 39-49.

63. Use of an MMRN2 portion according to any of claims 1-3 and 7-24, an agent according to any of claims 4-6, a fusion protein according to claim 25, an antibody according to any of claims 26-29, a nucleic acid molecule according to claim 30 or 50, a vector according to claim 31 or 51, a cell according to any of claims 32, 52, 53 and 55, a compound according to any of claims 33-38, or a CAR according to any of claims 39-49 in the preparation of a medicament for combating a disease or condition in an individual selected from the group consisting of cancer, psoriasis, menorrhagia, endometriosis, arthritis (both inflammatory and rheumatoid), macular degeneration, Paget's disease, retinopathy and its vascular complications (including proliferative and of prematurity, and diabetic retinopathy), benign vascular proliferations, fibroses, obesity and inflammation.

64. An MMRN2 portion according to any of claims 1-3 and 7-24, an agent according to any of claims 4-6, a fusion protein according to claim 25, an antibody according to any of claims 26-29, a nucleic acid molecule according to claim 30 or 50, a vector according to claim 31 or 51, a cell according to any of claims 32, 52, 53 and 55, a compound according to any of claims 33-38, or a CAR according to any of claims 39-49 for use in the preparation of a medicament for combating a disease or condition in an individual selected from the group consisting of cancer, psoriasis, menorrhagia, endometriosis, arthritis (both inflammatory and rheumatoid), macular degeneration, Paget's disease, retinopathy and its vascular complications (including proliferative and of prematurity, and diabetic retinopathy), benign vascular proliferations, fibroses, obesity and inflammation.

65. A method of targeting a cytotoxic moiety to neovasculature in the body of an individual, the method comprising:
administering to the individual a compound comprising (i) a MMRN2 portion according to any of claims 1-3 and 7-24; and (ii) a cytotoxic moiety.

66. A compound comprising (i) a MMRN2 portion according to any of claims 1-3 and 7-24; and (ii) a cytotoxic moiety, for use in targeting a cytotoxic moiety to neovasculature in the body of an individual.

67. Use of a compound comprising (i) a MMRN2 portion according to any of claims 1-3 and 7-24; and (ii) a cytotoxic moiety, in the preparation of a medicament for targeting a cytotoxic moiety to neovasculature in the body of an individual.

68. A method according to any of claims 59, 62 and 65, or a use according to any of claims 60, 61, 63, 64, 66 and 67, wherein at least one further anticancer agent and/or at least one further anti-angiogenesis agent is administered to the individual.

69. A method according to any of claims 59, 62, 65 and 68, or a use according to any of claims 60, 61, 63, 64, and 66-68, wherein the individual is one who is administered at least one further anticancer agent and/or at least one further anti-angiogenesis agent.

70. A method or use according to claim 68 or 69, wherein the at least one further anticancer agent is selected from cisplatin; carboplatin; 5-flurouracil; paclitaxel; mitomycin C; doxorubicin; gemcitabine; tomudex; pemetrexed; methotrexate; irinotecan, fluorouracil and leucovorin; oxaliplatin, 5-fluorouracil and leucovorin; and paclitaxel and carboplatin and/or wherein the at least one further anti-angiogenesis agent is bevacizumab (Avastin®).

71. A method of imaging neovasculature in the body of an individual the method comprising:
administering to the individual a compound comprising (i) an MMRN2 portion according to any of claims 1-3 and 7-24; and (ii) a detectable moiety, and
imaging the detectable moiety in the body.

72. A method according to claim 71 further comprising the step of detecting the location of the compound in the individual.

73. A method according to claim 71 or 72 wherein the detectable moiety comprises iodine-123, iodine-131, indium-111, fluorine-19, carbon-13, nitrogen-15, oxygen-17, technetium-99m, gadolinium, manganese or iron.

74. A method or use according to any of the preceding claims wherein the individual is a human.

75. A method or a use according to any of the preceding claims wherein the individual has a solid tumour.

76. A method or a use according to claim 75, wherein the solid tumour is a tumour of the colon, rectum, ovary, liver, bladder, prostate, breast, kidney, pancreas, stomach, oesophagus, lung or thyroid.

77. An ex vivo or in vitro method of inhibiting angiogenesis, the method comprising administering an MMRN2 portion according to any of claims 1-3 and 7-24 or an agent according to any of claims 4-6, or an antibody according to any of claims 26-29, to endothelial cells or to an angiogenesis model, ex vivo or in vitro.

78. A complex comprising:
a MMRN2 portion according to any of claims 1-3 and 7-24; and
(ii) (a) CLEC14A or a portion or variant thereof, said portion or variant being capable of binding to MMRN2, and/or
(b) CD93 or a portion or variant thereof, said portion or variant being capable of binding to MMRN2.

79. A kit of parts comprising:
(i) a MMRN2 portion according to any of claims 1-3 and 7-24; and
(ii) (a) CLEC14A or a portion or variant thereof, said portion or variant being capable of binding to MMRN2, and/or
(b) CD93 or a portion or variant thereof, said portion or variant being capable of binding to MMRN2.

80. A nucleic acid molecule capable of expressing:
(i) a MMRN2 portion according to any of claims 1-3 and 7-24; and
(ii) (a) CLEC14A or a portion or variant thereof, said portion or variant being capable of binding to MMRN2, and/or
(b) CD93 or a portion or variant thereof, said portion or variant being capable of binding to MMRN2.

81. A complex according to claim 78, a kit of parts according to claim 79, and a nucleic acid molecule according to claim 80, wherein the portion or variant of CLEC14A comprises or consists of the C-type lectin domain of CLEC14A or a variant thereof

82. A complex according to any of claim 78 or 81, a kit of parts according to claim 79 or 81, and a nucleic acid molecule according to claim 80 or 81, wherein the portion or variant of CLEC14A comprises or consists of a region of CLEC14A corresponding to the region spanning amino acid residues 97-108 of human CLEC14A or a variant thereof

83. A complex according to any of claims 78, 81 and 82, a kit of parts according to any of claims 79, 81 and 82, and a nucleic acid molecule according to any of claims 80-82, wherein the portion or variant of CD93 comprises or consists of the region corresponding to the region spanning amino acid residues 97-108 of human CD93 or a variant thereof.

84. A mutant MMRN2 polypeptide which has reduced binding to CLEC14A relative to wild type MMRN2.

85. A mutant MMRN2 polypeptide according to claim 84, wherein the mutant MMRN2, when compared to the corresponding wild type MMRN2, comprises one or more different amino acids in the region of MMRN2 corresponding to the region spanning amino acid residues 588-620 of human MMRN2.

86. A mutant MMRN2 polypeptide according to claim 84 or 85, wherein the mutant MMRN2 is a portion of MMRN2 consisting of the region of MMRN2 corresponding to the region spanning amino acid residues 495-603 or 487-603 or 604-674 of human MMRN2.

87. A mutant MMRN2 polypeptide according to any of claims 84-86, wherein the mutant MMRN2, when compared to the corresponding wild type MMRN2, has a different amino acid at a position which corresponds to any one or more of the following positions according to the numbering of the human MMNR2 as set out in FIG. 15: Leu-536, Val-575, Leu-578, Val-589, Leu-592, Phe-596, Leu-599, Leu-600, Asp-602, Ala-603, Arg-605, His-606, Leu-610, Leu-613, Gly-615, Glu-616, Glu-617, and Leu-658.

88. A mutant MMRN2 polypeptide according to any of claims 84-87, wherein the mutant MMRN2, when compared to the corresponding wild type MMRN2, has a different amino acid at a position which corresponds to any one or more of the following positions according to the numbering of the human MMNR2 as set out in FIG. 15: Leu-497, Glu-506, Gln-527, Val-540, Ala-546, Val-609, Glu-620, Gln-636, Ile-637, Leu-641, Leu-648 and Glu-666.

89. A mutant CLEC14A polypeptide which has reduced binding to MMRN2 relative to wild type CLEC14A, wherein the cysteine corresponding to cysteine-103 of human CLEC14A is mutated and/or the cysteine corresponding to cysteine-138 of human CLEC14A is mutated.

90. A mutant CD93 polypeptide which has reduced binding to MMRN2 relative to wild type CD93.

91. A mutant CD93 polypeptide according to claim 90, wherein the mutant CD93, when compared to the corresponding wild type CD93, comprises one or more different amino acids in the region of CD93 corresponding to the region spanning amino acid residues 97-108 of human CD93; and/or wherein, when compared to the corresponding wild type CD93, the cysteine corresponding to cysteine-104 of human CD93 is mutated and/or the cysteine corresponding to cysteine-136 of human CD93 is mutated.

92. A nucleic acid molecule encoding the mutant MMRN2 polypeptide of any of claims 84-88, the mutant CLEC14A polypeptide of claim 89, or the mutant CD93 polypeptide of claim 90 or 91.

93. A vector, such as an expression vector, comprising a nucleic acid molecule of claim 92.

94. A cell comprising the nucleic acid molecule of claim 92 or the vector of claim 93.

95. A kit of parts comprising: (i) an MMRN2 portion according to any of claims 1-3 and 7-24, an agent according to any of claims 4-6, a fusion protein according to claim 25, an antibody according to any of claims 26-29, a nucleic acid molecule according to claim 30 or 50, a vector according to claim 31 or 51, a cell according to any of claims 32, 52, 53 and 55, a compound according to any of claims 33-38, or a CAR according to any of claims 39-49, and (ii) at least one additional anti-cancer agent and/or at least one additional anti-angiogenic agent.

96. A kit of parts comprising: (i) an MMRN2 portion according to any of claims 1-3 and 7-24, an agent according to any of claims 4-6, a fusion protein according to claim 25, an antibody according to any of claims 26-29, a nucleic acid molecule according to claim 30 or 50, a vector according to claim 31 or 51, a cell according to any of claims 32, 52, 53 and 55, a compound according to any of claims 33-38, or a CAR according to any of claims 39-49; and
(ii) a cytotoxic moiety, optionally wherein the cytotoxic moiety is as defined in claim 33 or 34.

97. A kit of parts comprising (i) an MMRN2 portion according to any of claims 1-3 and 7-24, an agent according to any of claims 4-6, a fusion protein according to claim 25, an antibody according to any of claims 26-29, a nucleic acid molecule according to claim 30 or 50, a vector according to claim 31 or 51, a cell according to any of claims 32, 52, 53 and 55, a compound according to any of claims 33-38, or a CAR according to any of claims 39-49; and (ii) a detectable moiety, optionally wherein the detectable moiety is as defined in claim 30 or 31.

98. A method of identifying a portion of MMRN2 or a variant thereof, which portion may be useful in modulating angiogenesis or in combating cancer, or a lead compound for the identification of an agent that may be useful in modulating angiogenesis or in combating cancer, the method comprising:
providing CLEC14A or a portion or variant thereof, said portion or variant being capable of binding to MMRN2;
providing a candidate portion of MMRN2 or a variant thereof; and
determining whether the candidate portion modulates binding of CLEC14A or the portion or variant thereof, to MMRN2, or a portion or variant thereof, said portion or variant being capable of binding to CLEC14A.

99. A method according to claim 98, wherein the method further comprises:
determining whether the candidate portion of MMRN2 or variant thereof,
modulates binding of CD93, or a portion or variant thereof, said portion or variant being capable of binding to MMRN2,
to MMRN2 or a portion or variant thereof, said portion or variant being capable of binding to CD93.

100. A method of identifying an agent that may be useful in modulating angiogenesis or in combating cancer, or a lead compound for the identification of an agent that may be useful in modulating angiogenesis or in combating cancer, the method comprising:
providing CD93 or a portion or variant thereof, said portion or variant being capable of binding to MMRN2;
providing a candidate agent; and
determining whether the candidate agent modulates binding of CD93 or the portion or variant thereof, to MMRN2, or a portion or variant thereof, said portion or variant being capable of binding to CD93;
optionally wherein the candidate agent is an antibody, a peptide, a peptidomimetic, a natural product, a carbohydrate, an aptamer or a small molecule.

101. A method according to claim 100, wherein the method further comprises:
determining whether the candidate agent
modulates binding of CLEC14A, or a portion or variant thereof, said portion or variant being capable of binding to MMRN2,
to MMRN2 or a portion or variant thereof, said portion or variant being capable of binding to CLEC14A.

102. A method according to any of claims 98-101, further comprising the step of testing the candidate portion or agent in an angiogenesis assay.

103. A method for preparing an anticancer compound or anti-angiogenesis compound that may be useful in the treatment of a solid tumour, the method comprising identifying a compound using the method according to any of claims 98-102, and synthesising, purifying and/or formulating the identified compound.
